(19) United States
(12) Patent Application Publication
(10) Pub. No.: US 2022/0211835 A1
(43) Pub. Date: Jul. 7, 2022

(54) Replication Deficient Adenoviral Vectors for HIV Vaccine Applications

(71) Applicants: THE WISTAR INSTITUTE, Philadelphia, PA (US); SKM Pharma Tech Inc., Hatfield, PA (US)

(72) Inventors: Hildegund C.J. Ertl, Philadelphia, PA (US); Xiang Yang Zhou, Philadelphia, PA (US); Xiaoping Lo, Hatfield, PA (US)

(21) Appl. No.: 17/604,329

(22) PCT Filed: Oct. 2, 2019
(86) PCT No.: PCT/US19/54301

§ 371 Date: Oct. 15, 2021

Related U.S. Application Data
(60) Provisional application No. 62/835,108, filed on Apr. 17, 2019.

(51) Int. Cl. A61K 39/12; A61K 35/761; C12N 15/86; A61P 31/18

(52) U.S. Cl. CPC A61K 39/12; A61K 35/761; A61K 2039/53; A61P 31/18; C12N 15/86

(57) ABSTRACT

The invention includes compositions and methods of generating a chimpanzee-derived adenovirus serotype AdC6 or AdC7 vector vaccine, wherein an early gene E1 genomic region is deleted, and wherein the nucleic acid sequence further comprises an expression cassette comprising a promoter sequence operably linked to a sequence encoding a heterologous protein, wherein the heterologous protein is at least one HIV protein selected from the group consisting of gp140 and Gag, wherein gp140 is from a Chinese HIV clade selected from the group consisting of B, AE, BC and C, and wherein Gag is from a Chinese HIV clade B. Furthermore, the invention encompasses a pharmaceutical composition for vaccinating a mammal as well as a protein expression system.

### CROSS-REFERENCE TO RELATED APPLICATION

The present application claims priority under 35 U.S.C. § 119(e) to U.S. Provisional Patent Application No. 62/835,108 filed Apr. 17, 2019, which is incorporated herein by reference in its entirety.

### BACKGROUND OF THE INVENTION

HIV infection is prevalent worldwide, spurring a quest to develop efficient vaccines to treat or prevent HIV infection. The situation is also true in China and Asia. Vaccination is widely recognized as the most effective method of preventing or ameliorating morbidity from infectious diseases. Viral vector vaccines, such as those based on adenoviral vectors, may be used against various infectious and malignant diseases (Small and Ertl, Curr Opin Virol. 2011, October 1; 1(4): 241-245).

There is a need in the art for methods of producing more efficient adenovirus vector vaccine systems for treating or preventing HIV infection. The need is especially pressing in China and other countries in Asia. The present invention fulfills this need.

### SUMMARY

Provided is a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter operably linked to a sequence encoding a heterologous protein, wherein the heterologous protein is at least one HIV protein selected from the group consisting of gp140 and Gag;
wherein gp140 is from a Chinese HIV clade selected from the group consisting of B, AE, BC and C; and
wherein Gag is from a Chinese HIV clade B.

In some embodiments, the expression cassette is in the early gene E1 genomic region. In some embodiments, the expression cassette comprises a chimeric intron and/or CMV enhancer. In some embodiments, the early gene E3 genomic region consisting of ORF3, ORF4, ORF5, ORF6, and ORF7 is deleted. In further embodiments, the entire early gene E3 genomic region is deleted.

In some embodiments, the promoter is a constitutive promoter. In further embodiments, the promoter is a cytomegalovirus immediate early promoter (CMV).

In some embodiments, the nucleic acid sequence comprises SEQ ID NOs: 6 or 7.

Provided is a protein expression system comprising the the composition of any one of the previous embodiments, wherein the nucleic acid sequence comprises SEQ ID NOs: 6 or 7.

Also provided is a protein expression system comprising the composition of any one of the preceding embodiments, wherein the heterologous protein encoded by the expression cassette comprises an amino acid sequence selected from the group consisting of SEQ ID NOs: 1-5.

Provided is a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a constitutive promoter operably linked to a sequence encoding a heterologous protein, wherein the expression cassette is in the early gene E1 genomic region, wherein the heterologous protein is at least one HIV protein selected from the group consisting of gp140 and Gag;
wherein gp140 is from a Chinese HIV clade selected from the group consisting of B, AE, BC and C; and
wherein Gag is from a Chinese HIV clade B.

In some embodiments, the nucleic acid sequence comprises SEQ ID NOs: 6 or 7.

Provided is a protein expression system comprising the composition of any one of the preceding embodiments, wherein the heterologous protein encoded by the expression cassette comprises an amino acid sequence selected from the group consisting of SEQ ID NOs: 1-5.

Provided is a method of eliciting an immune response in a mammal against a heterologous protein, the method comprising administering to the mammal a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter operably linked to a sequence encoding a heterologous protein, wherein the heterologous protein is at least one HIV protein selected from the group consisting of gp140 and Gag;
wherein gp140 is from a Chinese HIV clade selected from the group consisting of B, AE, BC and C; and
wherein Gag is from a Chinese HIV clade B.

In some embodiments, the expression cassette is in the early gene E1 region. In some embodiments, the expression cassette comprises a chimeric intron and/or CMV enhancer.

In some embodiments, an early gene E3 genomic region consisting of ORF3, ORF4, ORF5, ORF6, and ORF7 is deleted. In further embodiments, the entire early gene E3 genomic region is deleted.

In some embodiments, the promoter is a constitutive promoter. In further embodiments, the promoter is a cytomegalovirus immediate early promoter (CMV).

In some embodiments, the nucleic acid sequence comprises SEQ ID NOs: 6 or 7.

Provided is a method of treating and/or preventing HIV in a mammal, the method comprising administering a therapeutically effective amount of a composition encoded by a nucleic acid sequence comprising SEQ ID NOs: 6 or 7.

Also provided is a method of vaccinating a mammal against HIV infection, the method comprising administering to the mammal a therapeutically effective amount of the the composition of any one of the previous embodiments, wherein administration of the composition elicits an immune response in the mammal. In some embodiments, the composition is administered prophylactically to the mammal. In further embodiments, the composition is administered therapeutically to the mammal. In yet further embodiments, the composition is administered in combination with an adjuvant.

Provided is a method of generating an effector and memory T cell immune response to a heterologous protein in a mammal, the method comprising the steps of: (a) administering the composition of any one of the previous embodiments to a mammal in an amount effective to elicit an immune response in the mammal; (b) administering a second effective amount of the composition of any one of the previous embodiments at a second, subsequent time period, wherein T memory cells directed against the heterologous protein are reactivated in the mammal. In some embodiments, the composition administered first in (a) and second in (b) comprises a same or a different HIV heterologous protein selected from the group consisting of gp140 and Gag. In further embodiments, the composition administered first in (a) and second in (b) is a same or a different serotype selected from the group consisting of AdC6 and AdC7. In yet further embodiments, the composition administered first in (a) and second in (b) is of a same or a different HIV Clade.

In some embodiments, the method further comprises the step of administering an immunogen to the mammal. In some embodiments, the immunogen comprises a heterologous protein, wherein the heterologous protein is at least one HIV protein selected from the group consisting of gp140 and Gag; wherein gp140 is from a Chinese HIV Clade selected from the group consisting of B, AE, BC and C; and wherein Gag is from a Chinese HIV clade B, wherein a B cell immune response is further augmented.

Provided is a method of generating an adaptive B cell immune response to a heterologous protein in a mammal, the method comprising the steps of: (a) administering the composition of any one of the previous embodiments to a mammal in an amount effective to elicit an immune response in the mammal; (b) administering a second effective amount of the composition of any one of the previous embodiments at a second, subsequent time period, wherein B memory cells directed against the heterologous protein are reactivated in the mammal. In some embodiments, the composition administered first in (a) and second in (b) comprises a same or a different HIV heterologous protein selected from the group consisting of gp140 and Gag. In further embodiments, the composition administered first in (a) and second in (b) has a same or a different serotype selected from the group consisting of AdC6 and AdC7. In yet further embodiments, the composition administered first in (a) and second in (b) is of a same or a different HIV Clade.

In some embodiments, the method further comprises the step of administering an immunogen to the mammal. In some embodiments, the immunogen comprises a heterologous protein, wherein the heterologous protein is at least one HIV env protein selected from any Clade from any source, wherein the B cell immune response is further augmented.

In some embodiments, the mammal is a human.

Any and all features of the aspects or embodiments may be combined to achieve new embodiments.

### BRIEF DESCRIPTION OF THE DRAWINGS

For the purpose of illustrating the invention, there are depicted in the drawings certain embodiments of the invention. However, the invention is not limited to the precise arrangements and instrumentalities of the embodiments depicted in the drawings.

FIG. 1A is a series of graphs illustrating percentage of CD8⁺CD44⁺ cells over all CD8⁺CD44⁺ cells from blood releasing cytokines in response to gag peptide. Background responses without the gag peptide were subtracted. Lines show mean responses ±SD. Line with stars above indicates a significant difference (p<0.01). FIG. 1B is series of graphs illustrating percentage of CD8⁺CD44⁺ cells over all CD8⁺CD44⁺ cells from pooled blood of mice immunized 14 days earlier with 10¹¹ virus particles (vp) of the AdC6gag or AdC7gag vectors producing cytokines in response to a peptide carrying the immunodominant epitope of gag. Background responses without gag peptide were subtracted.

FIG. 2 is series of graphs illustrating percentage of specific CD8⁺CD44⁺ cells over all CD8⁺CD44⁺ cells tested from spleens of individual mice 18 days after their immunization with 10¹¹ virus particles (vp) of the AdC6gag or AdC7gag vectors producing the indicated cytokines in response to gag peptide. The sum reflects the total response calculated based on Boolean gating. Background responses without gag peptide were subtracted.

FIGS. 3A-3B are a series of graphs showing T cell responses tested from pooled blood 14 days after immunization with 10¹⁰ or 10⁹ vp of the AdC6gag vector. The graph layout mirrors that of FIG. 1. FIG. 3A shows CD8⁺ T cell responses, FIG. 3B shows CD4⁺ T cell responses.

FIG. 4 shows ELISA results obtained with serum samples harvested and tested after priming with 10¹¹ vp of the indicated vectors on plates coated with gp140 protein of Clade C, AE or BC. Circles—mice immunized with AdC6 vectors. Squares—mice immunized with AdC7 vectors. Values obtained with sera from naïve mice were subtracted. Lines show medians. nt—not tested.

FIG. 5 shows ELISA results obtained with serum samples harvested and tested after priming with 10¹¹ vp of the indicated vectors followed by a boost with 10⁹ vp of the heterologous vectors expressing the same insert on plates coated with gp140 protein of Clade C, AE or BC. Circles—mice immunized with AdC6 and the AdC7 vectors. Squares—mice immunized with AdC7 and then boosted with AdC6 vectors. Values obtained with sera from naïve mice were subtracted. Lines show medians. nt—not tested.

FIG. 6 shows ELISA results obtained with serum samples harvested and tested after priming with 10¹¹ vp of the AdC6 vectors followed by a boost with 10⁹ vp of the AdC7 vectors expressing the same insert and then a second boost with a Clade C protein in alum on plates coated with gp140 protein of Clade C, AE or BC. Values obtained with sera from naïve mice were subtracted. Lines show medians.

FIG. 7 shows ELISA results obtained with serum samples harvested and tested after priming with 10¹¹ vp of the AdC6 vectors (circles) and after a boost with 10⁹ vp of the AdC7 vectors (squares) expressing the same insert on plates coated with gp140 protein of Clade C, AE or BC. Values obtained with sera from naïve mice were subtracted. Lines show medians. These data are similar to those in FIGS. 4 and 5 but the assays for the 2 time points were conducted simultaneously to allow for a direct comparison.

FIG. 8 shows ELISA results obtained with serum samples harvested and tested after priming with 10¹¹ vp of the AdC7 vectors (circles) and after a boost with 10⁹ vp of the AdC6 vectors (squares) expressing the same insert on plates coated with gp140 protein of Clade C, AE or BC. Values obtained with sera from naïve mice were subtracted. Lines show medians. These data are similar to those in FIGS. 4 and 5 but the assays for the 2 time points were conducted simultaneously to allow for a direct comparison.

FIG. 9 shows ELISA results obtained with serum samples harvested and tested after priming with 10¹¹ vp of the AdC6 vectors followed by boosting with 10⁹ vp of the AdC7 vectors (squares) expressing the same insert and then a second boost with a Clade C protein in alum on plates coated with gp140 protein of Clade C, AE or BC. Values obtained with sera from naïve mice were subtracted. Lines show medians. Lines with stars above indicate significant differences by 2-way Anova. These data are similar to those in FIGS. 5 and 6 but the assays for the 2 time points were conducted simultaneously to allow for a direct comparison.

FIG. 10 shows a combination of the data shown in FIGS. 6-8.

FIG. 11 shows adsorbance values of the different sera from individual mice group correlated according to the insert used for immunization against the three different Clades (C, AE and BC) used for testing. The Figure shows r-values. Significant values are indicated by stars above the bars.

FIG. 12 shows frequencies of gag-specific CD8+ T cells 2 weeks after priming AdC6gag or AdC7gag vectors tested with pooled blood (left) and after a boost with the heterologous vector tested 2 weeks later using PBMCs from individual mice. The experiment was controlled using PBMCs from naïve mice. Results show the sum of all cytokines (IFN-gamma, IL-2, granzyme B and TNF-alpha) calculated upon Boolean gating.

FIG. 13 shows frequencies of gag-specific CD8+ T cells 2 weeks after priming with the AdC6gag (left) and 4 weeks after a boost with the AdC7gag vector. Results show the sum of all cytokine (IFN-gamma, IL-2, granzyme B and TNF-alpha) calculated upon Boolean gating.

FIG. 14 shows antibody responses as adsorbance against clade C env after priming of BALB/c mice with a mixture of the different AdC6gp140 vectors given each at 10⁹ or 10¹⁰ vp followed 6 weeks later by a boost with the AdC7gp140 vectors given at the same doses followed 6 weeks later by a Clade C env protein boost. The experiment was controlled by sera from naïve BALB/c mice.

FIG. 15 shows antibody responses as adsorbance against clade C, AE and BC env after priming of ICR mice with a mixture of the different AdC6gp140 or AdC7gp140 vectors given each at 10¹⁰ vp followed 8 weeks later by a boost with the heterologous vectors vector given at the same doses. The experiment was controlled by sera from naïve ICR mice.

### DETAILED DESCRIPTION OF THE INVENTION

The present invention relates to compositions and methods for generating a chimpanzee-derived adenovirus vector comprising a nucleic acid sequence comprising a deletion in some of the adenovirus early genes (i.e. wherein an early gene E1 region is deleted, and wherein in some embodiments ORF3, ORF4, ORF5, ORF6, and ORF7 from early gene E3 or the entire E3 gene are also deleted) and a promoter sequence linked to a sequence encoding a heterologous protein comprising, in certain embodiments, an HIV protein selected from the group consisting of gp140 and Gag; wherein gp140 is from a Chinese HIV clade selected from the group consisting of B, AE, BC and C; and wherein Gag is from a Chinese HIV clade B. Additionally, the current invention includes compositions and methods of treating of and/or preventing or immunizing against, a specific disease or disorder, and methods of inducing an effector and memory T and B cell immune response in a mammal administered the chimpanzee-derived adenovirus vector the invention.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

The term “antibody” or “Ab” as used herein, refers to a protein, or polypeptide sequence derived from an immunoglobulin molecule, which specifically binds to a specific epitope on an antigen. Antibodies can be intact immunoglobulins derived from natural sources or from recombinant sources and can be immunoreactive portions of intact immunoglobulins. The antibodies useful in the present invention may exist in a variety of forms including, for example, polyclonal antibodies, monoclonal antibodies, intracellular antibodies (“intrabodies”), Fv, Fab and F(ab)₂, as well as single chain antibodies (scFv) and humanized antibodies (Harlow et al., 1998, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). An antibody may be derived from natural sources or from recombinant sources. Antibodies are typically tetramers of immunoglobulin molecules.

The term “ameliorating” or “treating” means that the clinical signs and/or the symptoms associated with a disease are lessened as a result of the actions performed. The signs or symptoms to be monitored will be well known to the skilled clinician.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods. The term “biological” or “biological sample” refers to a sample obtained from an organism or from components (e.g., cells) of an organism. The sample may be of any biological tissue or fluid. Frequently the sample will be a “clinical sample” which is a sample derived from a patient. Such samples include, but are not limited to, bone marrow, cardiac tissue, sputum, blood, lymphatic fluid, blood cells (e.g., white cells), tissue or fine needle biopsy samples, urine, peritoneal fluid, and pleural fluid, or cells therefrom. Biological samples may also include sections of tissues such as frozen sections taken for histological purposes.

As used herein, “greater” refers to expression levels which are at least 10% or more, for example, 20%, 30%, 40%, or 50%, 60%, 70%, 80%, 90% higher or more, and/or 1.1 fold, 1.2 fold, 1.4 fold, 1.6 fold, 1.8 fold, 2.0 fold higher or more, and any and all whole or partial increments therebetween, than a control.

As used herein, the terms “control,” or “reference” are used interchangeably and refer to a value that is used as a standard of comparison.

The term “immunogenicity” as used herein, refers to the innate ability of an antigen or organism to elicit an immune response in an animal when the antigen or organism is administered to the animal. Thus, “enhancing the immunogenicity” refers to increasing the ability of an antigen or organism to elicit an immune response in an animal when the antigen or organism is administered to an animal. The increased ability of an antigen or organism to elicit an immune response can be measured by, among other things, a greater number of antibodies that bind to an antigen or organism, a greater diversity of antibodies to an antigen or organism, a greater number of T-cells specific for an antigen or organism, a greater cytotoxic or helper T-cell response to an antigen or organism, a greater expression of cytokines in response to an antigen, and the like.

As used herein, the terms “eliciting an immune response” or “immunizing” refer to the process of generating a B cell and/or a T cell response against a heterologous protein.

The term “activation”, as used herein, refers to the state of a cell following sufficient cell surface moiety ligation to induce a noticeable biochemical or morphological change. Within the context of T cells, such activation refers to the state of a T cell that has been sufficiently stimulated to induce cellular proliferation. Activation of a T cell may also induce cytokine production and performance of regulatory or cytolytic effector functions. Within the context of other cells, this term infers either up or down regulation of a particular physico-chemical process.

The term “activated T cell” means a T cell that is currently undergoing cell division, cytokine production, performance of regulatory or cytolytic effector functions, and/or has recently undergone the process of “activation.”

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

“Heterologous antigens” used herein to refer to an antigen that is not endogenous to the organism comprising or expressing an antigen. As an example, a virus vaccine vector comprising or expressing a viral or tumor antigen comprises a heterologous antigen. The term “Heterologous protein” as used herein refers to a protein that elicits a beneficial immune response in a subject (i.e. mammal), irrespective of its source.

By the terms “Human Immunodeficiency Virus” or HIV” as used herein is meant any HIV strain or variant that is known in the art or that is heretofore unknown, including without limitation, HIV-1 and HIV-2. HIV-1 is exemplified in certain embodiments disclosed herein.

The term “specifically binds”, “selectively binds” or “binding specificity” refers to the ability of the humanized antibodies or binding compounds of the invention to bind to a target epitope with a greater affinity than that which results when bound to a non-target epitope. In certain embodiments, specific binding refers to binding to a target with an affinity that is at least 10, 50, 100, 250, 500, or 1000 times greater than the affinity for a non-target epitope.

As used herein, by “combination therapy” is meant that a first agent is administered in conjunction with another agent. “In combination with” or “In conjunction with” refers to administration of one treatment modality in addition to another treatment modality. As such, “in combination with” refers to administration of one treatment modality before, during, or after delivery of the other treatment modality to the individual. Such combinations are considered to be part of a single treatment regimen or regime.

“Humoral immunity” or “humoral immune response” both refer to B-cell mediated immunity and are mediated by highly specific antibodies, produced and secreted by B-lymphocytes (B-cells).

“Prevention” refers to the use of a pharmaceutical compositions for the vaccination against a disorder.

“Adjuvant” refers to a substance that is capable of potentiating the immunogenicity of an antigen. Adjuvants can be one substance or a mixture of substances and function by acting directly on the immune system or by providing a slow release of an antigen. Examples of adjuvants are aluminium salts, polyanions, bacterial glycopeptides and slow release agents as Freund's incomplete.

“Delivery vehicle” refers to a composition that helps to target the antigen to specific cells and to facilitate the effective recognition of an antigen by the immune system. The best-known delivery vehicles are liposomes, virosomes, microparticles including microspheres and nanospheres, polymeres, bacterial ghosts, bacterial polysaccharides, attenuated bacteria, virus like particles, attenuated viruses and ISCOMS.

As used herein, the term “expression cassette” means a nucleic acid sequence capable of directing the transcription and/or translation of a heterologous coding sequence. In some embodiments, the expression cassette comprises a promoter sequence operably linked to a sequence encoding a heterologous protein. In some embodiments, the expression cassette further comprises at least one regulatory sequence operably linked to the sequence encoding the heterologous protein.

“Incorporated into” or “encapsulated in” refers to an antigenic peptide that is within a delivery vehicle, such as microparticles, bacterial ghosts, attenuated bacteria, virus like particles, attenuated viruses, ISCOMs, liposomes and preferably virosomes.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that may comprise a protein or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

A “fusion protein” as used herein refers to a protein wherein the protein comprises two or more proteins linked together by peptide bonds or other chemical bonds. The proteins can be linked together directly by a peptide or other chemical bond, or with one or more amino acids between the two or more proteins, referred to herein as a spacer.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “RNA” as used herein is defined as ribonucleic acid.

“Transform”, “transforming”, and “transformation” is used herein to refer to a process of introducing an isolated nucleic acid into the interior of an organism.

The term “treatment” as used within the context of the present invention is meant to include therapeutic treatment as well as prophylactic, or suppressive measures for the disease or disorder. As used herein, the term “treatment” and associated terms such as “treat” and “treating” means the reduction of the progression, severity and/or duration of a disease condition or at least one symptom thereof. The term ‘treatment’ therefore refers to any regimen that can benefit a subject. The treatment may be in respect of an existing condition or may be prophylactic (preventative treatment). Treatment may include curative, alleviative or prophylactic effects. References herein to “therapeutic” and “prophylactic” treatments are to be considered in their broadest context. The term “therapeutic” does not necessarily imply that a subject is treated until total recovery. Similarly, “prophylactic” does not necessarily mean that the subject will not eventually contract a disease condition. Thus, for example, the term treatment includes the administration of an agent prior to or following the onset of a disease or disorder thereby preventing or removing all signs of the disease or disorder. As another example, administration of the agent after clinical manifestation of the disease to combat the symptoms of the disease comprises “treatment” of the disease.

The term “equivalent,” when used in reference to nucleotide sequences, is understood to refer to nucleotide sequences encoding functionally equivalent polypeptides. Equivalent nucleotide sequences will include sequences that differ by one or more nucleotide substitutions, additions- or deletions, such as allelic variants; and will, therefore, include sequences that differ from the nucleotide sequence of the nucleic acids described herein due to the degeneracy of the genetic code.

The term “isolated” as used herein with respect to nucleic acids, such as DNA or RNA, refers to molecules separated from other DNAs or RNAs, respectively that are present in the natural source of the macromolecule. The term isolated as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments, which are not naturally occurring as fragments and would not be found in the natural state. The term “isolated” is also used herein to refer to polypeptides, which are isolated from other cellular proteins and is meant to encompass both purified and recombinant polypeptides. An “isolated cell” or “isolated population of cells” is a cell or population of cells that is not present in its natural environment.

A “mutation” as used therein is a change in a DNA sequence resulting in an alteration from its natural state. The mutation can comprise a deletion and/or insertion and/or duplication and/or substitution of at least one deoxyribonucleic acid base such as a purine (adenine and/or thymine) and/or a pyrimidine (guanine and/or cytosine). Mutations may or may not produce discernible changes in the observable characteristics (phenotype) of an organism.

As used herein, the term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides. ESTs, chromosomes, cDNAs, mRNAs, and rRNAs are representative examples of molecules that may be referred to as nucleic acids.

As used herein, “operably linked” sequences include both expression control sequences that are contiguous with the gene of interest and expression control sequences that act in trans or at a distance to control the gene of interest. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. There are numerous expression control sequences, including promoters which are native, constitutive, inducible and/or tissue-specific, are known in the art that may be used in the compositions of the invention. “Operably linked” should be construed to include RNA expression and control sequences in addition to DNA expression and control sequences.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence, which is required for expression of a gene product operably linked to the promoter/regulatory sequence. In some instances, this sequence may be the core promoter sequence and in other instances, this sequence may also include an enhancer sequence and other regulatory elements, which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

As used herein, the term “pharmaceutical composition” refers to a mixture of at least one compound useful within the invention with other chemical components, such as carriers, stabilizers, diluents, adjuvants, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The language “pharmaceutically acceptable carrier” includes a pharmaceutically acceptable salt, pharmaceutically acceptable material, composition or carrier, such as a liquid or solid filler, diluent, excipient, solvent or encapsulating material, involved in carrying or transporting a compound(s) of the present invention within or to the subject such that it may perform its intended function. Typically, such compounds are carried or transported from one organ, or portion of the body, to another organ, or portion of the body. Each salt or carrier must be “acceptable” in the sense of being compatible with the other ingredients of the formulation, and not injurious to the subject. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; diluent; granulating agent; lubricant; binder; disintegrating agent; wetting agent; emulsifier; coloring agent; release agent; coating agent; sweetening agent; flavoring agent; perfuming agent; preservative; antioxidant; plasticizer; gelling agent; thickener; hardener; setting agent; suspending agent; surfactant; humectant; carrier; stabilizer; and other non-toxic compatible substances employed in pharmaceutical formulations, or any combination thereof. As used herein, “pharmaceutically acceptable carrier” also includes any and all coatings, antibacterial and antifungal agents, and absorption delaying agents, and the like that are compatible with the activity of the compound, and are physiologically acceptable to the subject. Supplementary active compounds may also be incorporated into the compositions.

As used herein, the term “effective amount” or “therapeutically effective amount” means the amount of the virus like particle generated from vector of the invention which is required to prevent the particular disease condition, or which reduces the severity of and/or ameliorates the disease condition or at least one symptom thereof or condition associated therewith.

A “subject” or “patient,” as used therein, may be a human or non-human mammal. Non-human mammals include, for example, livestock and pets, such as ovine, bovine, porcine, canine, feline and murine mammals. Preferably, the subject is human.

“Titers” are numerical measures of the concentration of a virus or viral vector compared to a reference sample, where the concentration is determined either by the activity of the virus, or by measuring the number of viruses in a unit volume of buffer. The titer of viral stocks are determined, e.g., by measuring the infectivity of a solution or solutions (typically serial dilutions) of the viruses, e.g., on HeLa cells using the soft agar method (see, Graham & van der Eb (1973) Virology 52:456-467) or by monitoring resistance conferred to cells, e.g., G418 resistance encoded by the virus or vector, or by quantitating the viruses by UV spectrophotometry (see, Chardonnet & Dales (1970) Virology 40:462-477).

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. In the present disclosure, the term “vector” includes an autonomously replicating virus.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Description

Provided is a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter sequence operably linked to a sequence encoding a heterologous protein, wherein the heterologous protein is at least one HIV protein selected from the group consisting of gp140 and Gag; wherein gp140 is from a Chinese HIV clade selected from the group consisting of B, AE, BC and C; and wherein Gag is from a Chinese HIV clade B.

In some embodiments, the expression cassette further comprises at least one regulatory sequence operably linked to the sequence encoding the heterologous protein.

In some embodiments, the expression cassette is in the early gene E1 genomic region.

In some embodiments, the expression cassette further comprises a chimeric intron and/or CMV enhancer.

In some embodiments, an early gene E3 genomic region consisting of ORF3, ORF4, ORF5, ORF6, and ORF7 is deleted.

In some embodiments, the entire early gene E3 genomic region is deleted.

In further embodiments, the promoter is a constitutive promoter. In yet further embodiments, the promoter is a cytomegalovirus immediate early promoter (CMV).

In some embodiments, the nucleic acid sequence comprises SEQ ID NOs: 6 or 7. In some embodiments, the nucleic acid sequence consists of SEQ ID NOs: 6 or 7.

Provided is a protein expression system comprising the composition of any one of the previous embodiments, wherein the nucleic acid sequence comprises SEQ ID NOs: 6 or 7. Also provided is a protein expression system comprising the composition of any one of the previous embodiments, wherein the nucleic acid sequence consists of SEQ ID NOs: 6 or 7. Also provided is a protein expression system comprising the composition of any one of the previous embodiments, wherein the heterologous protein encoded by the expression cassette comprises an amino acid sequence selected from the group consisting of SEQ ID NOs: 1-5.

Also provided is a method of eliciting an immune response in a mammal against a heterologous protein, the method comprising administering to the mammal a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter sequence operably linked to a sequence encoding a heterologous protein, wherein the heterologous protein is at least one HIV protein selected from the group consisting of gp140 and Gag; wherein gp140 is from a Chinese HIV Clade selected from the group consisting of B, AE, BC and C; and wherein Gag is from a Chinese HIV clade B.

In some embodiments, the expression cassette further comprises at least one regulatory sequence operably linked to the sequence encoding the heterologous protein.

In some embodiments, the expression cassette is in the early gene E1 genomic region.

In some embodiments, the expression cassette further comprises a chimeric intron and/or CMV enhancer.

In some embodiments, an early gene E3 genomic region consisting of ORF3, ORF4, ORF5, ORF6, and ORF7 is deleted.

In some embodiments, the entire early gene E3 genomic region is deleted.

In further embodiments, the promoter is a constitutive promoter. In yet further embodiments, the promoter is a cytomegalovirus immediate early promoter (CMV).

Provided is a method of treating and/or preventing HIV in a mammal, the method comprising administering a therapeutically effective amount of a composition encoded by a nucleic acid sequence comprising SEQ ID NOs: 6 or 7. In some embodiments, the nucleic acid sequence consists of SEQ ID NOs: 6 or 7.

Provided is a method of vaccinating a mammal against HIV infection, the method comprising administering to the mammal a therapeutically effective amount of the composition of any one of the previous embodiments, wherein administration of the composition elicits an immune response in the mammal. In some embodiments, the composition is administered prophylactically to the mammal. In further embodiments, the composition is administered therapeutically to the mammal. In yet further embodiments, the composition is administered in combination with an adjuvant.

Provided is a method of generating a effector and memory T cell immune response to a heterologous protein in a mammal, the method comprising the steps of: (a) administering the composition of any one of the previous embodiments to a mammal in an amount effective to elicit an immune response in the mammal; (b) administering a second effective amount of the composition of any one of the previous embodiments at a second, subsequent time period, wherein T memory cells directed against the heterologous protein are reactivated in the mammal. In some embodiments, the composition administered first in (a) and second in (b) comprises a same or a different HIV heterologous protein selected from the group consisting of gp140 and Gag; wherein gp140 is from a Chinese HIV Clade selected from the group consisting of B, AE, BC and C; and wherein Gag is from a Chinese HIV clade B. In further embodiments, the composition administered first in (a) and in (b) is a same or a different serotype selected from the group consisting of AdC6 and AdC7.

Provided is a method of generating an adaptive B cell immune response to a heterologous protein in a mammal, the method comprising the steps of: (a) administering the composition of any one of the previous embodiments to a mammal in an amount effective to elicit an immune response in the mammal; (b) administering a second effective amount of the composition of any one of the previous embodiments at a second, subsequent time period, wherein B memory cells directed against the heterologous protein are reactivated in the mammal.

In some embodiments, the method further comprises the step of administering an immunogen to the mammal. In further embodiments, the immunogen comprises a heterologous protein, wherein the heterologous protein is at least one HIV protein selected from gp140 derived from any Clade from any source, wherein a B cell immune response is further augmented. In some embodiments, the heterologous protein is from a Chinese Clade or from an African Clade. In some embodiments, the heterologous protein so administered is the same heterologous protein that is expressed in the nucleic acid sequence of a chimpanzee-derived adenovirus vector of any one of the previous embodiments. In some embodiments, the heterologous protein so administered is the same heterologous protein that was administered in step (a) and/or step (b) of any one of the previous methods. In some embodiments, the immunogen further comprises an adjuvant, for example alum.

In some embodiments, the immunogen is administered to the mammal after steps (a) and (b).

In some embodiments, the mammal is a human.

Adenoviral vectors comprising deletions in E1 and/or E3 are disclosed in International Application PCT/US2017/043315 (WO 2018/026547), which is incorporated herein in its entirety.

Vaccine compositions comprising adenovirus particles made using the adenovirus vectors disclosed herein can be used to induce immunity in a mammal against one or more encoded heterologous proteins or antigenic portions thereof. Immunity can be induced using the disclosed vaccine compositions or dosage units. Immune responses can be assessed using suitable methods known in the art, as disclosed, for example, in WO2012/02483.

### Heterologous Gene Expression

In one aspect, although the cytomegalovirus immediate early promoter is exemplified herein as the promoter driving expression of the HIV protein, the invention should not be construed to be limited to this promoter sequence. Promoter sequences that are useful in the invention include any promoter that induces high levels of gene expression. Such promoters may include, but are not limited to those disclosed elsewhere herein.

In one embodiment, a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence, which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

In some embodiments, the invention further includes the use of a tissue-specific promoter that drives expression of a given heterologous gene in one or more specific types of cells (e.g., myoglobin promoter, muscle creatine kinase promoter, desmin promoter, mammalian troponin 1 promoter, and skeletal alpha-action promoter). Furthermore, any artificial synthetic promoters known in the art can be used in this invention as these promoters can provide optimal efficiency and stability for the heterologous gene. Additionally, enhancer sequences regulate expression of the gene contained within a vector. Typically, enhancers are bound with protein factors to enhance the transcription of a gene. Enhancers may be located upstream or downstream of the gene it regulates. Enhancers may also be tissue-specific to enhance transcription in a specific cell or tissue type.

In order to assess the expression of the heterologous gene of interest, the expression vector to be introduced into a cell can also contain either a selectable marker gene or a reporter gene or both to facilitate identification and selection of expressing cells from the population of cells sought to be infected through the hybrid-virus vectors. In other aspects, the selectable marker may be carried on a separate piece of DNA and used in a co-infection/transfection procedure. Both selectable markers and reporter genes may be flanked with appropriate regulatory sequences to enable expression in the host cells. Useful selectable markers include, for example, antibiotic-resistance genes, such as the neomycin resistant gene and the like.

Reporter genes are used for identifying potentially infected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82).

It will be apparent to one skilled in the art that the invention is not limited to the nature of the heterologous gene that is expressed by the adenovirus vector of the invention. Any suitable heterologous gene can be used where expression of the gene provides a benefit to the mammal. For example, the heterologous gene may be a viral protein whose expression in a mammal confers immunity to infection by the virus. Similarly, the heterologous gene may be a bacterial antigen, a parasitic antigen, a fungal antigen, a cancer antigen, an antigen involved in a deleterious autoimmune reaction, or any other protein where an immune response directed thereto provides benefit.

### Heterologous Proteins

In the present invention, the adenovirus vector of the invention may encode a heterologous protein, wherein the heterologous protein is at least one HIV protein selected from the group consisting of gp140 and Gag, wherein gp140 is from a Chinese HIV clade selected from the group consisting of B, AE, BC and C, and wherein Gag is from a Chinese HIV clade B. Typically, the heterologous protein is a peptide fragment, polypeptide, protein or fusion protein. Optionally, the heterologous protein is suitable such that cell-mediated immune and humoral responses are induced against it in a mammal following administration of the vector to the mammal.

### Methods of the Invention

The vectors of the invention are useful in a variety of applications useful for immunizing a mammal against disease, and/or treating, preventing or diminishing risk of disease in a mammal.

The invention therefore includes a method of immunizing a mammal against a heterologous protein. The method comprises administering to the mammal a composition comprising a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter sequence operably linked to a sequence encoding a heterologous protein, wherein the heterologous protein is at least one HIV protein selected from the group consisting of gp140 and Gag, wherein gp140 is from a Chinese HIV clade selected from the group consisting of B, AE, BC and C, and wherein Gag is from a Chinese HIV clade B, and wherein expression of the heterologous protein induces an immune response in the mammal.

In some embodiments, the expression cassette further comprises at least one regulatory sequence operably linked to the sequence encoding the heterologous protein.

In some embodiments, the expression cassette is in the early gene E1 genomic region.

In some embodiments, the expression cassette further comprises a chimeric intron and/or CMV enhancer.

In some embodiments, an early gene E3 genomic region consisting of ORF3, ORF4, ORF5, ORF6, and ORF7 is deleted.

In some embodiments, the entire early gene E3 genomic region is deleted.

In one embodiment the chimpanzee-derived Ad vector is AdC6. In one embodiment, the AdC6 has Genbank accession number AY530877. In one embodiment the chimpanzee-derived Ad vector is AdC7. In one embodiment, the AdC7 has Genbank accession number AY530878.

The invention further includes a method of treating a mammal in need thereof where the method administering a therapeutically effective amount of a composition encoded by a chimpanzee-derived adenovirus vector comprising a nucleic acid sequence comprising SEQ ID NOs: 6 or 7, wherein expression of the heterogeneous gene provides benefit to the mammal. In one aspect, the invention includes a method of generating effector and memory T cell immune responses to a heterologous protein in a mammal. In some embodiments, the nucleic acid sequence consists of SEQ ID NOs: 6 or 7. In another aspect, the invention includes a method of generating an adaptive B cell immune response to a heterologous protein in a mammal.

Additionally included in the invention is a method of diminishing the risk that a mammal will develop a disease. The method comprises administering to the mammal a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter sequence operably linked to a sequence encoding a heterologous protein, wherein the heterologous protein is at least one HIV protein selected from the group consisting of gp140 and Gag, wherein gp140 is from a Chinese HIV clade selected from the group consisting of B, AE, BC and C, and wherein Gag is from a Chinese HIV clade B.

In some embodiments, the expression cassette further comprises at least one regulatory sequence operably linked to the sequence encoding the heterologous protein.

In some embodiments, the expression cassette is in the early gene E1 genomic region.

In some embodiments, the expression cassette further comprises a chimeric intron and/or CMV enhancer.

In some embodiments, an early gene E3 genomic region consisting of ORF3, ORF4, ORF5, ORF6, and ORF7 is deleted.

In some embodiments, the entire early gene E3 genomic region is deleted.

Expression of the heterogeneous gene induces an immune response to the heterologous protein encoded thereby in the mammal, thereby diminishing the risk that the mammal will develop a disease (e.g. HIV-1) associated with the heterologous protein.

### Adenovirus Vector Production

Methods of making the adenovirus vector of the invention are described in detail in the Experimental Examples Section herein and in U.S. application Ser. No. 14/190,787 (U.S. Pat. No. 9,624,510) incorporated herein by reference. In general, production, purification and quality control procedures for adenovirus vectors are well established in the art. Once a vector backbone is created, molecular cloning can be used to create an adenoviral plasmid comprising a coding sequence for an antigenic heterologous protein. In some embodiments, the plasmid can be transfected into packaging cells that provide E1 of a suitable adenovirus serotype in trans. Packaging cells are well known in the art, and cells lines such as HEK293 or PERC6 can be used for this purpose. Viral particles are then harvested once plaques become visible. Fresh cells can then be infected to ensure continued replication of the adenovirus. Quality can be assessed using Southern blotting or other methods, such as restriction enzyme mapping, sequencing, and PCR, to confirm the presence of the transgene and the lack of gene rearrangements or undesired deletions.

Vaccine compositions comprising adenovirus particles made using the adenovirus vectors disclosed herein can be used to induce immunity against the encoded antigenic protein. Vaccines can be formulated using standard techniques and can comprise, in addition to a replication-incompetent adenovirus vector encoding a desired protein, a pharmaceutically acceptable vehicle, such as phosphate-buffered saline (PBS) or other buffers, as well as other components such as antibacterial and antifungal agents, isotonic and absorption delaying agents, adjuvants, and the like. In some embodiments vaccine compositions are administered in combination with one or more other vaccines. Dosage units of vaccine compositions can be provided. Such dosage units typically comprise 10⁸ to 10¹¹ adenoviral particles (e.g., 10⁸, 5×10⁸, 10⁹, 5×10⁹, 10¹⁰, 5×10¹⁰, 10¹¹). In some embodiments, the dosage of 5×10¹⁰ virus particles is of choice. Particularly, this dosage (5×1010) suits best humans in clinical trials.

### Pharmaceutical Compositions and Formulations.

The vector of the invention may be formulated as a pharmaceutical composition.

Such a pharmaceutical composition may be in a form suitable for administration to a subject (i.e. mammal), or the pharmaceutical composition may further comprise one or more pharmaceutically acceptable carriers, one or more additional ingredients, or some combination of these. The various components of the pharmaceutical composition may be present in the form of a physiologically acceptable salt, such as in combination with a physiologically acceptable cation or anion, as is well known in the art.

In one embodiment, the pharmaceutical compositions useful for practicing the method of the invention may be administered to deliver a dose of between 10⁶ and 10¹² VP.

In one embodiment, the pharmaceutical compositions useful for practicing the method of the invention may comprise an adjuvant. Non-limiting examples of suitable are Freund's complete adjuvant, Freund's incomplete adjuvant, Quil A, Detox, ISCOMs or squalene.

Pharmaceutical compositions that are useful in the methods of the invention may be suitably developed for inhalation, oral, rectal, vaginal, parenteral, topical, transdermal, pulmonary, intranasal, buccal, ophthalmic, intrathecal, intravenous or another route of administration. Other contemplated formulations include projected nanoparticles, liposomal preparations, resealed erythrocytes containing the active ingredient, and immunologically-based formulations. The route(s) of administration is readily apparent to the skilled artisan and depends upon any number of factors including the type and severity of the disease being treated, the type and age of the veterinary or human patient being treated, and the like.

Although the descriptions of pharmaceutical compositions provided herein are principally directed to pharmaceutical compositions suitable for ethical administration to humans, it is understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

The composition of the invention may comprise a preservative from about 0.005% to 2.0% by total weight of the composition. The preservative is used to prevent spoilage in the case of exposure to contaminants in the environment.

### Administration/Dosing

The regimen of administration may affect what constitutes an effective amount. For example, the adenovirus vector of the invention may be administered to the subject (i.e. mammal) in a single dose, in several divided dosages, as well as staggered dosages may be administered daily or sequentially, or the dose may be continuously infused, or may be a bolus injection. Further, the dosages may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to treat the disease in the subject. An effective amount of the composition necessary to achieve the intended result will vary and will depend on factors such as the disease to be treated or prevented, the age, sex, weight, condition, general health and prior medical history of the subject being treated, and like factors well-known in the medical arts. In particular embodiments, it is especially advantageous to formulate the composition in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the composition and the heterologous protein to be expressed, and the particular therapeutic effect to be achieved.

### Routes of Administration

One skilled in the art will recognize that although more than one route can be used for administration, a particular route can provide a more immediate and more effective reaction than another route. Routes of administration of any of the compositions\ of the invention include inhalation, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

### Kits

In some embodiments a kit is provided for treating, preventing, or ameliorating an a given disease, disorder or condition, or a symptom thereof, as described herein wherein the kit comprises: a) a compound or compositions as described herein; and optionally b) an additional agent or therapy as described herein. The kit can further include instructions or a label for using the kit to treat, prevent, or ameliorate the disease, disorder or condition. In yet other embodiments, the invention extends to kits assays for a given disease, disorder or condition, or a symptom thereof, as described herein. Such kits may, for example, contain the reagents from PCR or other nucleic acid hybridization technology (microarrays) or reagents for immunologically based detection techniques (e.g., ELISpot, ELISA).

### EXAMPLES

The invention is now described with reference to the following Examples.

These Examples are provided for the purpose of illustration only and the invention should in no way be construed as being limited to these Examples, but rather should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The results of the experiments are now described in the following examples.

### Methods:

According to the Los Alamos database the most prevalent Clades of HIV-1 in China are A/E (29.2%), different types of B/C (30.1%) with mainly 07_B/C (18.7%), B (23.1%) and C (14.7%). Extensive database searches were performed and a panel of Envelope (env) sequences was assembled for induction of antibodies that would be candidates for the development of a comprehensive HIV-1 vaccine for China. In these searches, more recent Chinese isolates for which full-length sequences are available were focused on. Env sequences that carry a K in position 169 and a V in position 172, which, are crucial for binding of broadly neutralizing V2-specific antibodies and for their ADCC activity were preferentially selected. For Gag, a clade B sequence that contains an epitope that is crucial for screening of CD8+ T cell responses in experimental animals was selected.

### Example 1: Vector Construction and Initial Immunogenicity Testing

### First Generation Vectors

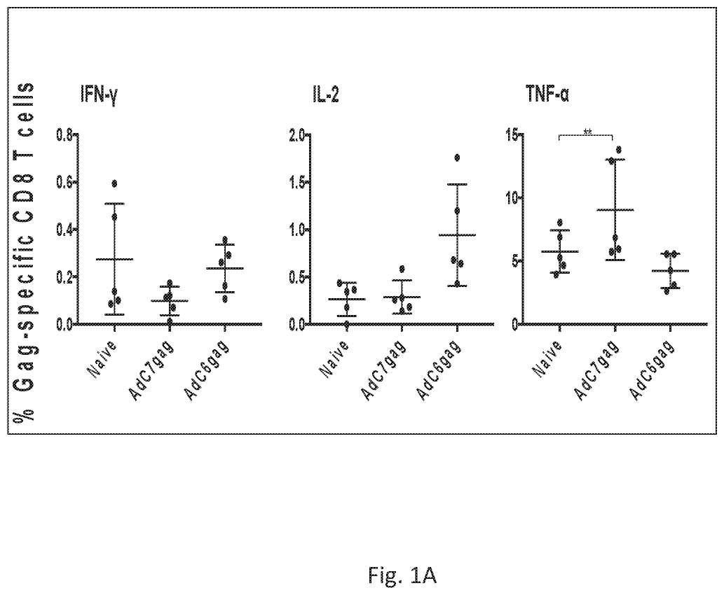

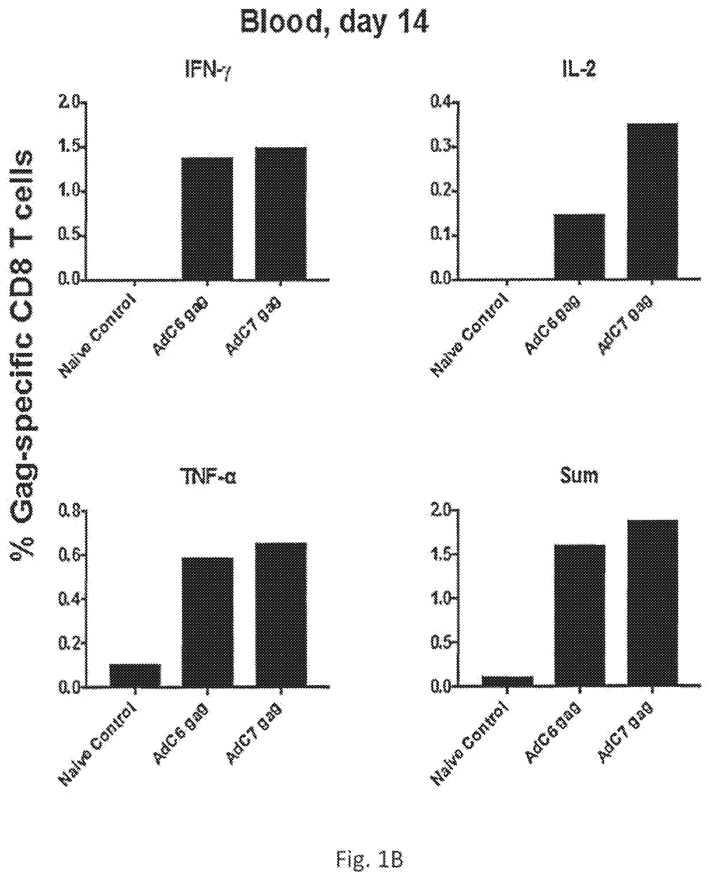

AdC6 and AdC7 vectors expressing gag of HIV clade B and gp140 of HIV clades B, AE, BC and C were generated using an expression cassette without intron and enhancer within E1- and partially E3-deleted vectors. Vectors were titrated for virus content. Vectors were shown to have genetic integrity and were genetically stable upon serial culture. Only the AdC7gp140BC vector induced a gp140-specific B cell response. (FIG. 1A)

### Second Generation Vectors

A second set of vectors were constructed using the same AdC backbones (but for AdC7gp140BC) and inserts but the expression cassette was changed by including an intron and enhancer within the expression cassette. Upon rescue, vectors were titrated, and genetic integrity was established. These vectors as shown below were found to be immunogenic.

Western blots were conducted for the gag vectors. The first generation gag vector failed to express detectable amounts of gag protein. The second generation gag vectors showed good expression. The Env vectors due to lack of specific antibodies gave ambiguous results. Mass spectrometry may be used to determine expression independent of antibodies, as was determined by use of one of the second generation vectors.

### Example 2: Immunogenicity of Second Generation Gag Vectors

Groups of BALB/c mice were immunized with 10¹¹ vp of the second generation gag vectors. Their pooled blood was tested 2 weeks later for CD8+ T cell responses by intracellular cytokine staining upon stimulation with the peptide carrying the immunodominant epitope of gag or upon sham stimulation as above (FIG. 1B). Mice immunized with either vector showed positive responses.

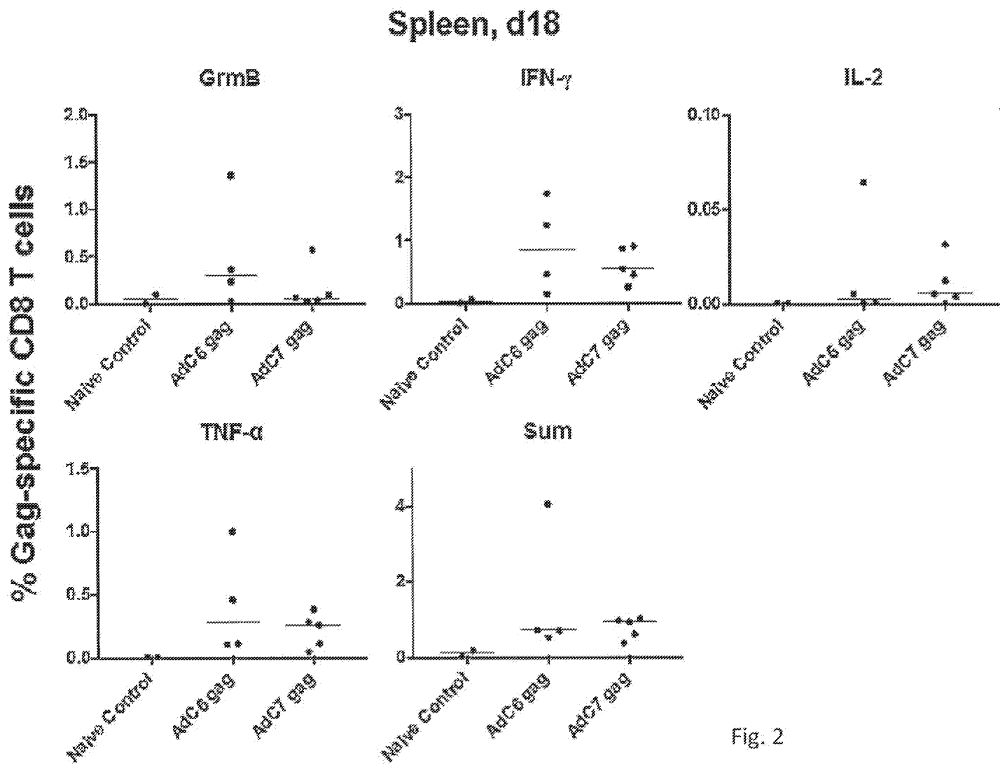

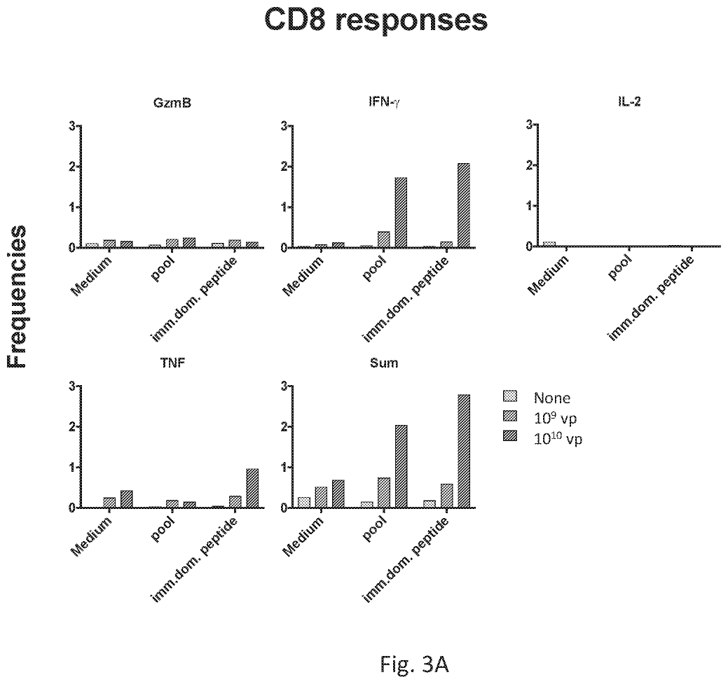

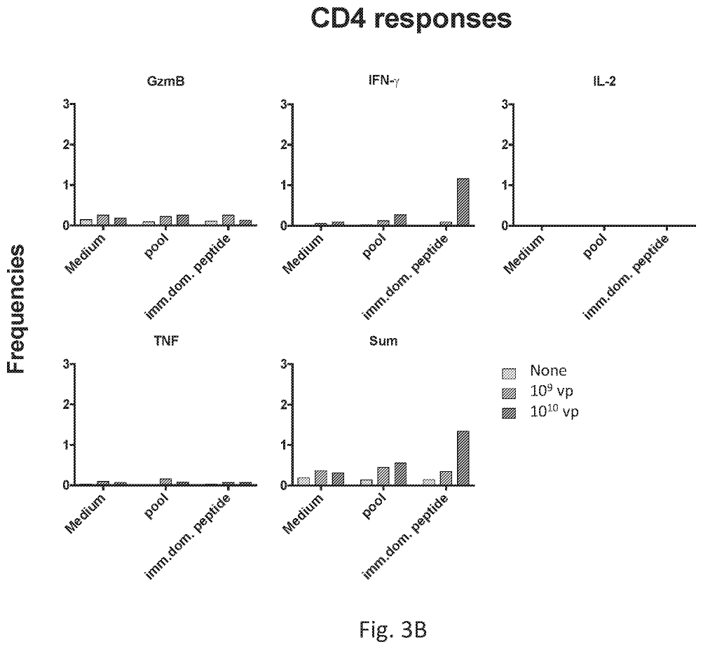

Splenocytes from individual mice were tested 3 days later including staining for interferon (IFN)-gamma, tumor necrosis factor (TNF)-alpha, interleukin (IL)-2 and granzyme B (GrmB) (FIG. 2). Upon vaccination, mice showed positive responses for multiple cytokines. The experiment was repeated using lower vector doses of 10⁹ and 10¹⁰ vp for the AdC6gag vector and again vectors at these doses induced a detectable CD8⁺ T cell response and as is typical for adenovirus vectors a more modest CD4⁺ T cell response (FIG. 3).

### Example 3: First Generation Gp140 Vectors

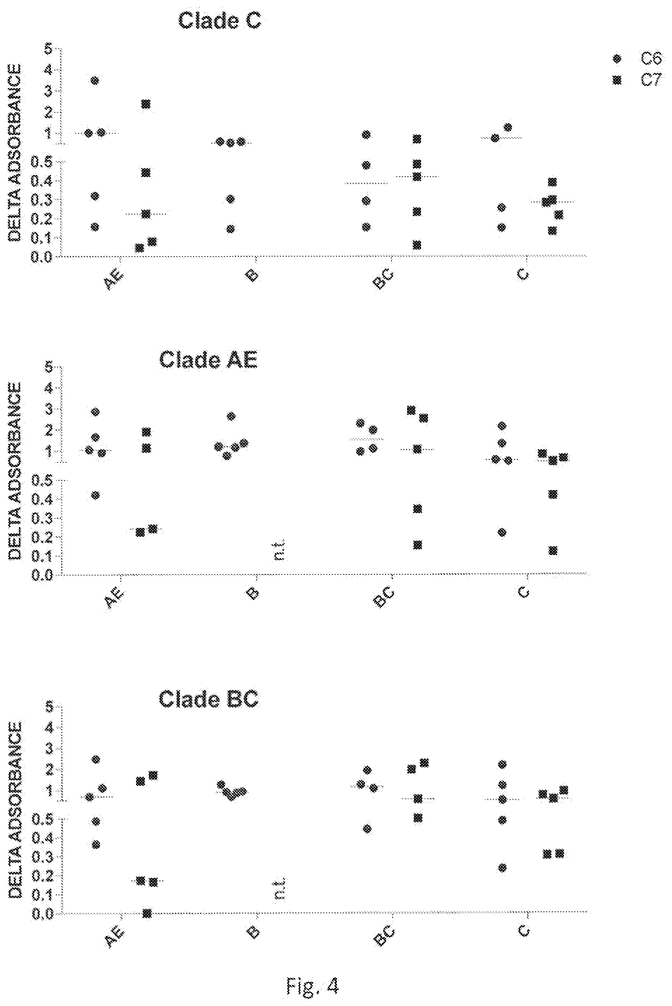

ICR mice were injected with 10¹¹ vp of the gp140 expressing vectors. They were bled 4 weeks later, and sera were tested by an ELISA on a baculovirus-derived gp140 (Clade C) or BSA coated plates in comparison to sera from naïve mice (negative control) or from mice injected with an already established gp140 vector (positive control). Mice immunized with the AdC7BC developed a detectable antibody response (FIG. 4). Some but not all of the AE immunized mice developed gp140-specific antibodies, and mice immunized with the other vectors failed to seroconvert.

### Example 4: Second Generation Gp140 Vectors

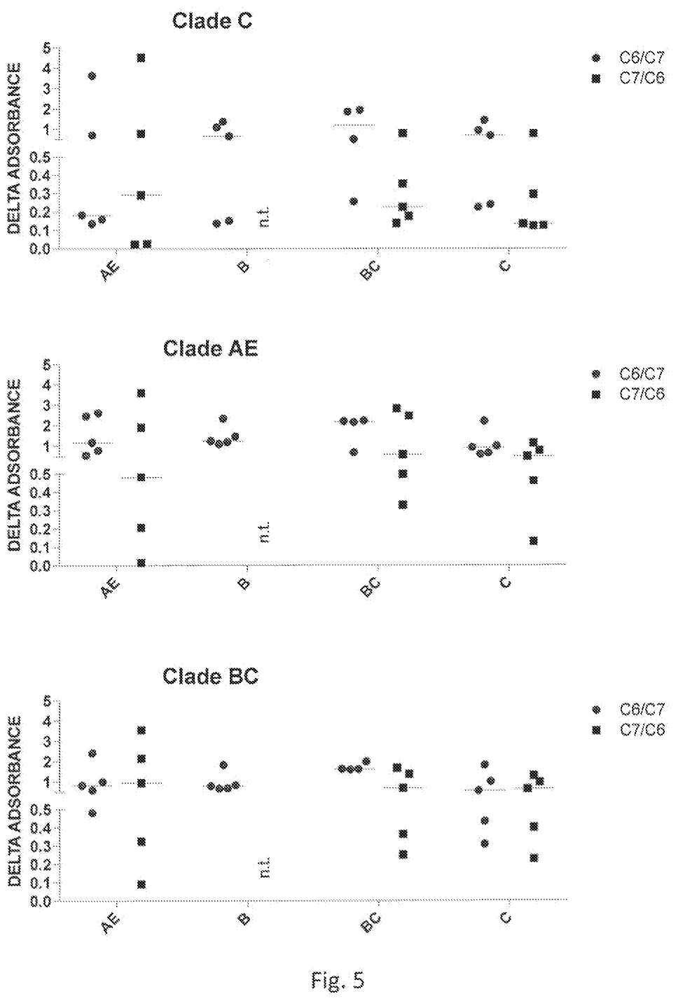

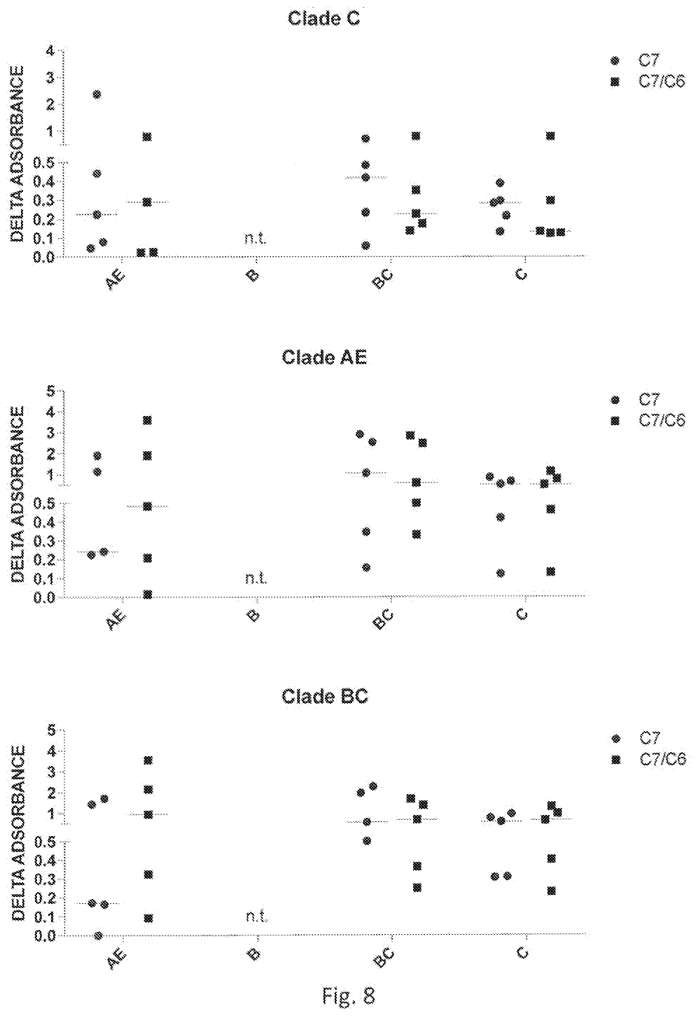

Vectors expressing gp140 (AdC6gp140AE, AdC6gp140B, AdC6gp140C, AdC6gp140BC, AdC7gp140AE, AdC7gp140B, AdC7gp140C), were generated using an expression cassette with intron and an enhancer. Upon titration vectors together with the 1^{st} generation AdC7gp140BC vector were injected at 10¹¹ vp into ICR mice. Their sera were tested 4 weeks later for antibodies to gp140 by ELISAs on plates coated with baculovirus-derived gp140 proteins derived from an early African HIV-1 clade C isolate, a Chinese HIV-1 clade AE isolate, and a Chinese HIV-1 clade BC isolate, the latter two match the sequences of the AdC insert. All vectors induced antibody responses to the 3 env proteins although not all mice responded (FIG. 4). Mice were boosted 5 weeks after priming with the heterologous vectors expressing the same inserts using a vector dose of 10⁹ vp per mouse. Individual sera were tested 4 weeks later on clade C, AE and BC gp140 proteins. As shown in FIG. 5 some of the non-responders became seropositive after the boost, which was most effective in enhancing responses that had been low after priming (e.g., after AdC6BC on clade C protein or AdC7AE on all proteins). The boost was relatively ineffective in some groups (FIG. 8), which may be attributed to the high vector dose used for priming and the 100-fold lower dose used for the boost.

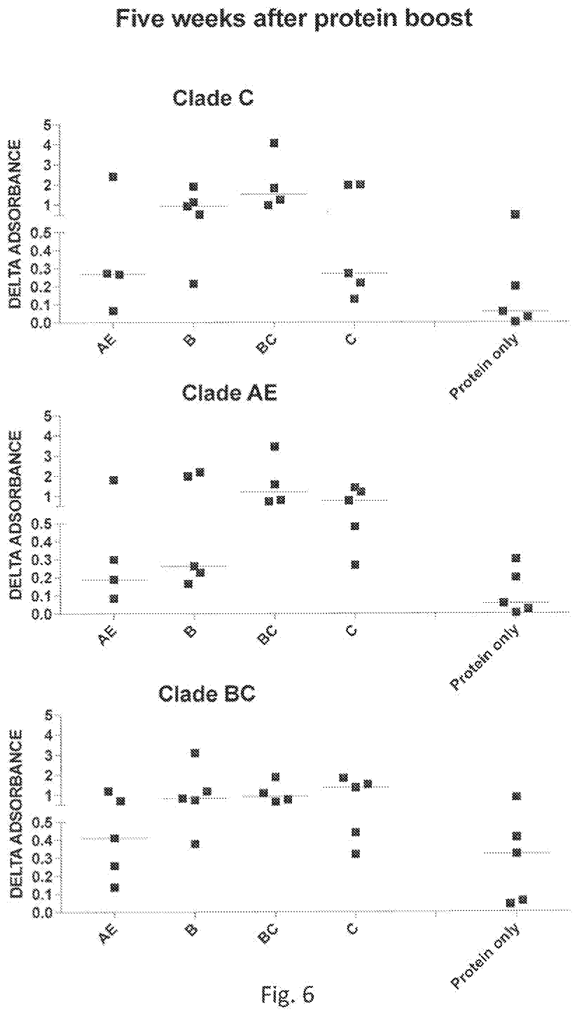

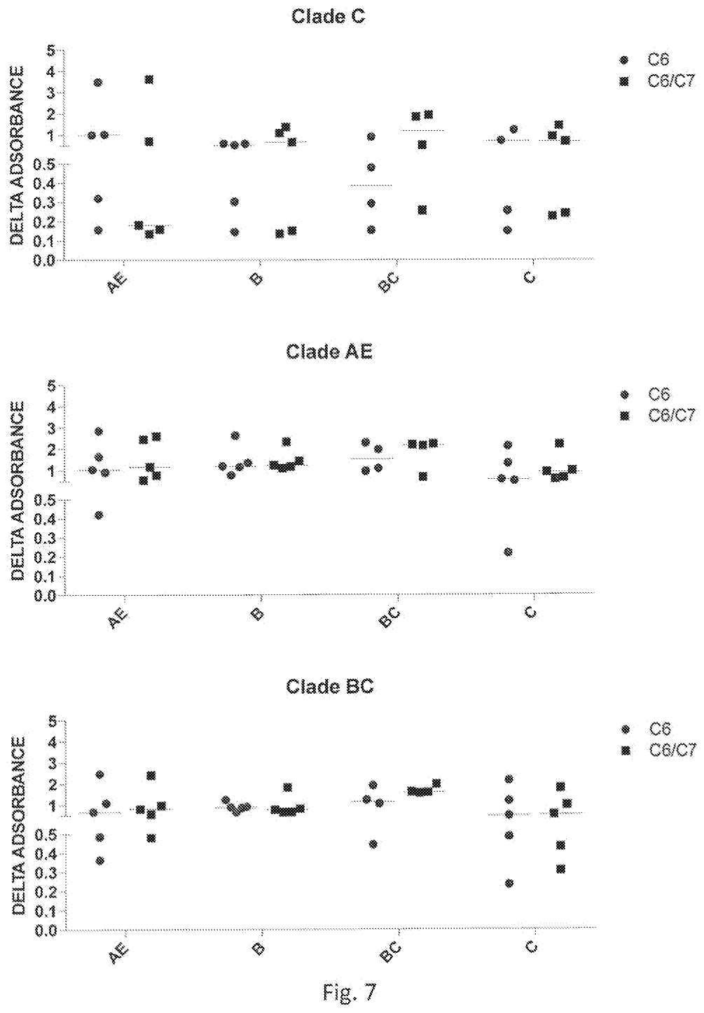

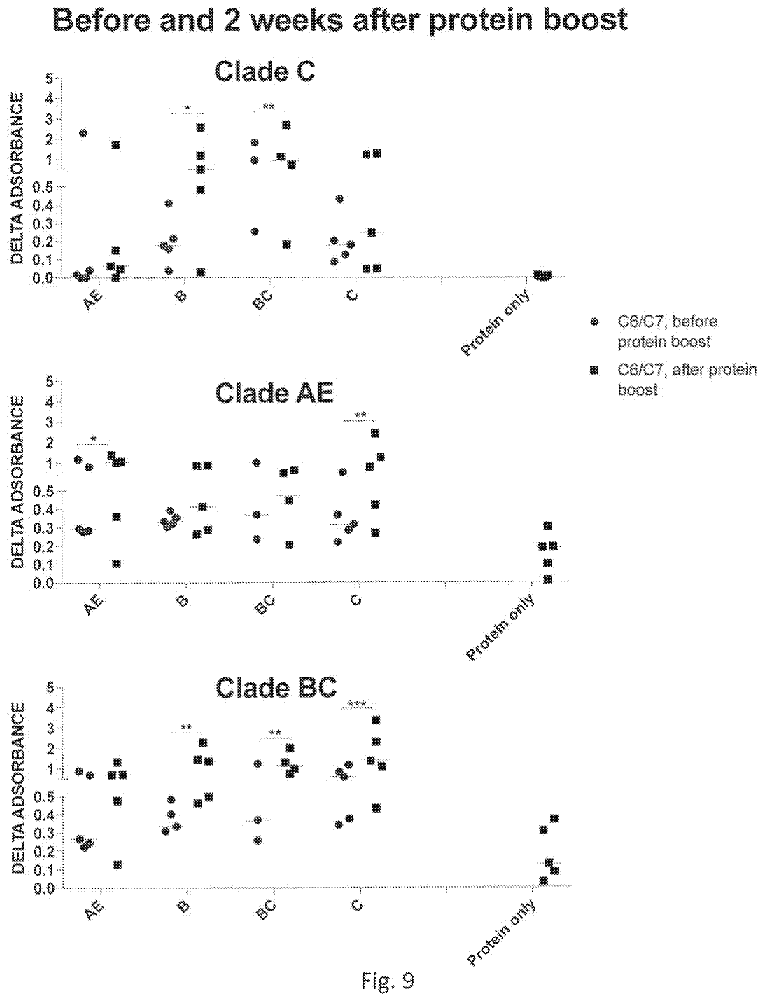

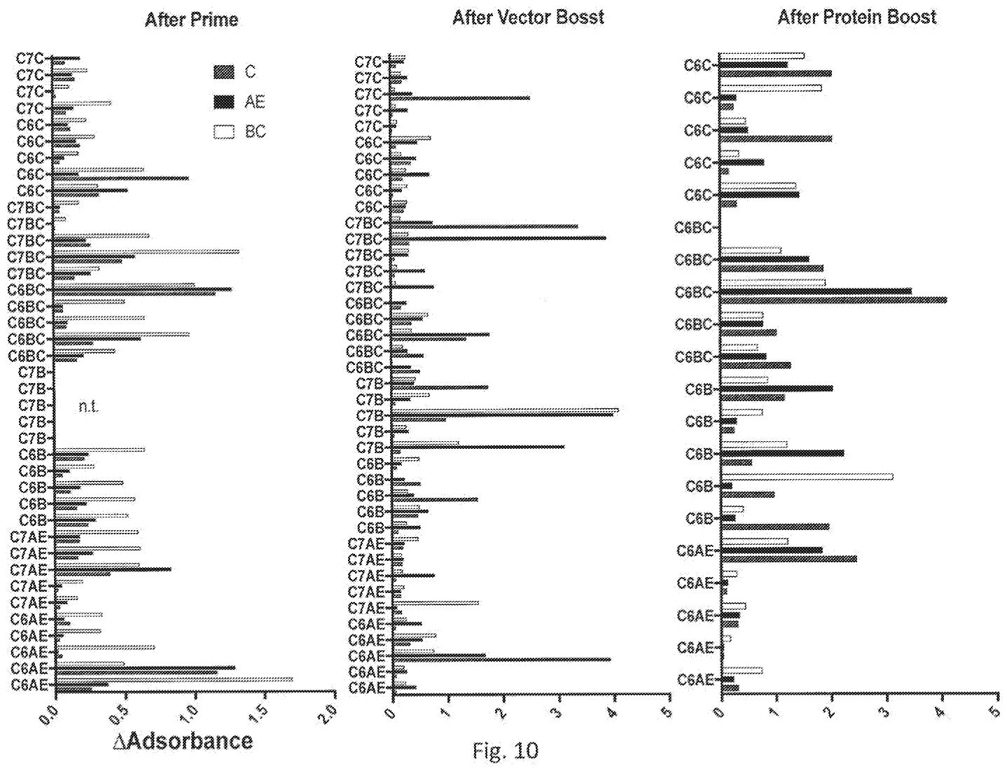

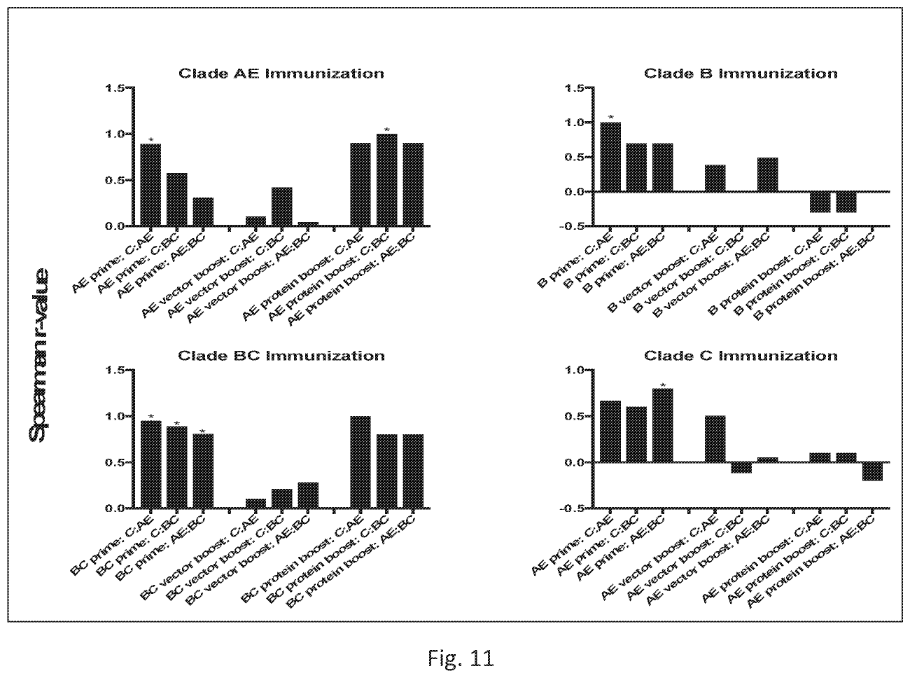

Mice primed with the AdC6 vectors and boosted with the AdC7 vectors were boosted again with 2 μg/mouse of a recombinant clade C gp140 protein from the AIDS Reagent Program (protein CN54) diluted 1:1 in alum. As shown in FIGS. 6 and 9 the protein was very effective at enhancing the vector primed antibody response so that by 5 weeks after this boost all but one mouse in the AE group showed robust antibody responses to the two gp140 derived from Chinese isolates. For comparison, naïve mice were immunized with the same protein in alum; some of these mice developed gp140-specific antibody responses but titers were well below those observed in vector primed mice (FIG. 9). Antibody titers tested on gp140 of the 3 different clades were compared. As shown in FIG. 10, responses differed depending on the protein they were tested on. Mice that had high antibody titers against gp140 of one clade did not necessarily have high titers to gp140 of the other clades. By the same token, the data obtained on plates coated with gp140 from the 3 different Clades showed relatively poor correlations (FIG. 11).

### Example 5: Prime-Boost Regimens

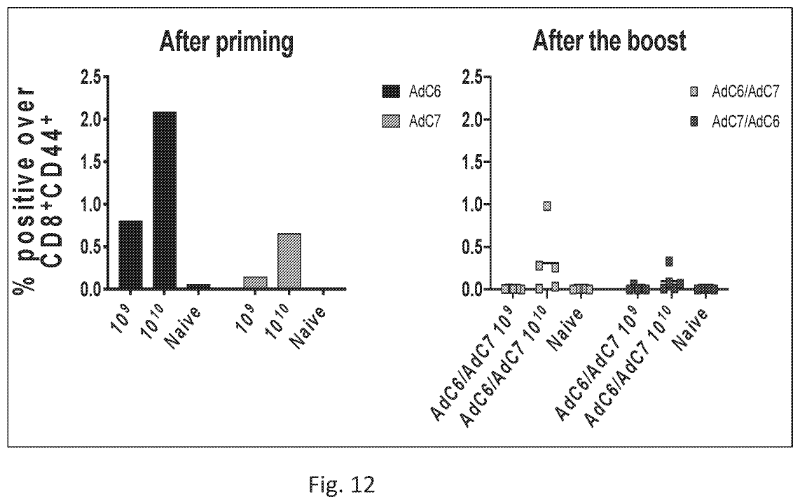

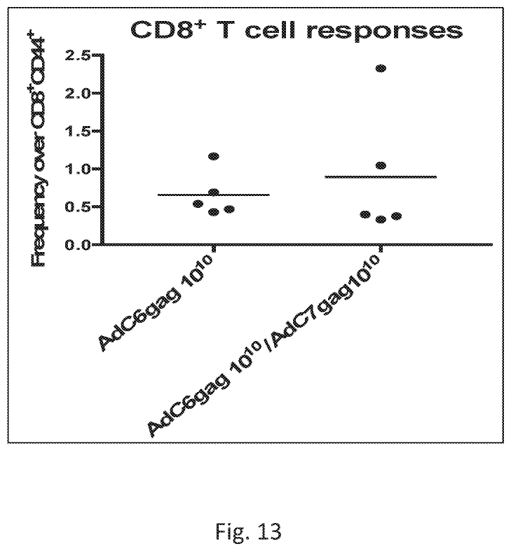

Several prime boost regimens were conducted with the AdCgag vectors. In the first set of experiments, priming was conducted with AdC6gag or AdC7gag at 10⁹ or 10¹⁰ vp and boosts were given 6 weeks later with heterologous vector given at the same dose. In a follow-up experiment, mixtures of gag and env vectors were used. Boosts were given 6 weeks after the prime. In both experiments, a CD8⁺ T cell response to gag was obtained after priming, which paradoxically declined after the boost. The results of the first experiment are shown in FIG. 12. Such results were previously obtained with other vectors, indicating that the CD8⁺ T cells had remained highly activated after the prime and were therefore susceptible to apoptosis upon re-encounter of their antigen upon the boost. The experiment was repeated using 10¹⁰ vp of Ad6gag for the prime and 10¹⁰ vp of AdC7 for the boost. In the follow-up experiment there was an iterval of 2 months between the prime and the boost. The result was more promising as a small increase in frequencies of gag-specific CD8⁺ T cells was observed (FIG. 13). However, frequencies were still well below those routinely seen after prime-boosting with a US-origin clade B gag, indicating that with this particular insert a longer waiting period between priming and boosting is most likely warranted.

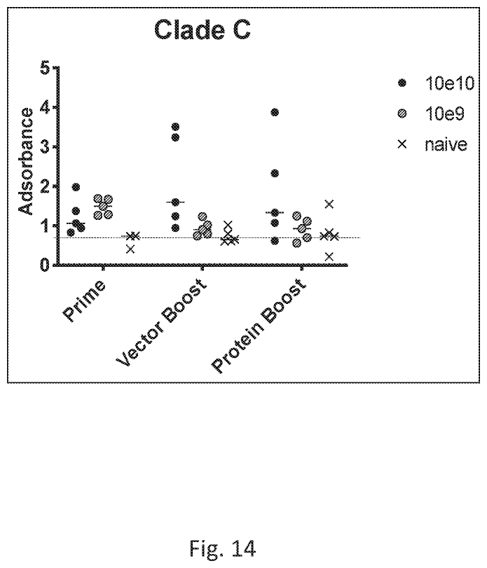

An experiment was conducted with the vector mixtures in BALB/c mice to assess antibody responses. All other antibody assays had been conducted in ICR mice. When sera against Clade AE and BC proteins were tested, the background responses in naïve mice were extreme high. Background responses against Clade C were less high but still substantial making it virtually impossible to assess if indeed a response had been achieved (FIG. 14).

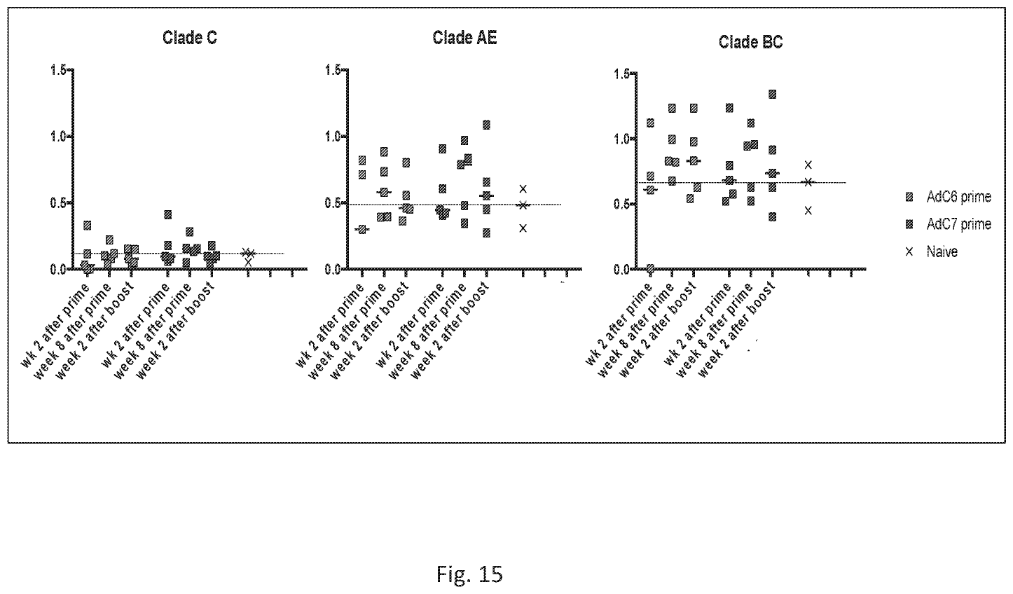

Mixtures of vectors expressing gp140 were tested in ICR mice. Mice were injected with a mixture of the AdC6gP140 Clade C, B, AE and BC vectors at 10¹⁰ vp per vector or with mixtures of the corresponding AdC7 vectors. Mice were bled 2 and 8 weeks later and were then boosted with the heterologous vectors, i.e., AdC6gp140 Clade B, C, AE, BC immune mice were boosted with the corresponding AdC7 vectors and vice versa. Mice were bled 2 weeks later. Antibodies to gp140 of Clade C, BC and AE were determined by ELISA as described elsewhere herein. Although antibody responses were seen in some mice, titers were not as robust as after immunization with vectors expressing gp140 of only one Clade. Furthermore, no increase was seen upon booster immunization. The results are shown in FIG. 15.

Sequences:
Gp140 Clade AE1: Accession number, JX112804.
SEQ ID NO: 1
MRVKGTQMNWPNLWKWGTLILGLVIMCSASDNLWVTVYYGVPVWRDANTTLFCASDAKAH
ETEVHNVWATYACVPTDPNPQEIPMENVTENFNMWKNNMVEQMQEDVISLWDQSLKPCVK
LTPLCVTLICTNANLTKINSTNSGPKVIGNVTDEVRNCSFNMTTLLTDKKQKVYALFYKL
DIVPIDNSNSSEYRLINCNTSVIKQACPKISFDPIPIHYCTPAGYAILKCNDKNFNGTGP
CKNVSSVQCTHGIKPVVSTQLLLNGSLAEEEIIIRSENLTNNAKTIIVHLNKAVEINCTR
PSNNTRTSIRIGPGQIFYRTGDIIGDIRQAYCEINGTKWNETLRQVAKKLKEQFNNTIKF
QPPSGGDLEITMLHFNCRGEFFYCNTTKLFNSTWERNETIKGGNGNGNDTIILPCRIKQI
INMWQGAGQAMYAPPISGIINCVSNITGILLTRDGGNTNETAEIFRPGGGNIKDNWRSEL
YKYKVVQIEPLGVAPTKAKLTVQARQLLSGIVQQQSNLLRAIEAQQHMLQLTVWGIKQLQ
ARILAVESYLKHQQFLGLWGCSNKIICTTAVPWNSSWSNKSYDEIWENMTWIEWEREIGN
YTNQIYDILTKSQEQQDKNEKELLELDQWASLWNWFSITKWLW*
Gp140 Clade B: Accession number, H1M215399.
SEQ ID NO: 2
MRVKGIRKNYQHLWRWGTMLLGMLMICSAAENLWVTVYYGVPVWKEATTTLFCASDAKAY
DTEVHNIWATHACVPTDPNPQEVVLGNVTENFNMWKNDMVEQMHEDIISLWDQSLKPCVK
LTPLCVTLNCTNLRNTNNTSSNTSNMTEGGEIKNCSFDITTSIRTKVKDYALFYELDIVA
IDNTSYRLRQCNTSVITQACPKISFEPIPIHYCTPAGFAILKCNNKTFNGTGPCTNVSTV
QCTHRIRPVVSTQLLLNGSLAEEEVVIRSSNFTDNAKVIIVQLKESVEINCTRPNNNTRK
SIPLGPGKAWYTTGQIIGDIRQAHCNLSRAKWENTLQQITKKLREQFGNKTIIFNQSSGG
DPEVVTHSFNCGGEFFYCNTSQLFNSTWYNNSTWNDTNDTTENSTITLPCRIKQIVNMWQ
EVGKAMYAPPIRGQIRCSSNITGLLLTRDGGKNESNTTETFRPGGGDMRDNWRSELYKYK
VVKIEPLGVAPTRAKLTVQARQLLSGIVQQQRNLLRAIEAQQHLLQLTVWGIKQLQARVL
AVERYLKDQQLLGIWGCSGKLICTTAVPWNVSWSNRSLSEIWDNMTWMEWEREIGNYTKQ
IYSLIEESQNQQEKNELELLEWDKWASLWNWFNITNWLW*
Gp140 Clade C: Accession number, KF835515.
SEQ ID NO: 3
MRVRGTQRNYPQWWIWGILGFWMLMICNVGGNLWVTVYYGVPVWKEATTTLFCASDAKAY
ENEVHNVWATHACVPTDPNPQEMVLENVTENFNMWKNEMVNQMHEDVISLWDQSLKPCVK
LTPLCVTLKCSNVTLKNNTVNSNETQYRKNCTFNTTTELKNRKQKVSAIFYRIDIVPLGN
ESSGNYRLINCNTSAITQACPKVSFDPIPIHYCTPAGYALLKCNNKTFNGTGPCNNVSTV
QCTHGIKPVVSTQLLLNGSLAEEEIIIRSENLTNNVKTIIVHLNESVEIVCIRPGNNTRQ
SIRIGPGQTFYAPGEIIGNIRQAHCNINGTKWNETLQGVGKKLAEHFPNKTIKFKPSSGG
DPEITTHSFNCRGEFFYCDTSGLFNSTYNSTYVPNGTESKPNITIQCRIKQIINMWQEVG
RAMYAPPIKGSITCKSNITGLLLVRDGGANTTEEIFRPGGGDMRDNWRSELYKYKVVEIK
PLGIAPTEAKLTVQARQLLSGIVQQQNNLLKAIEAQQHMLQLTVWGIKQLQTRVLAIERY
LKDQQLLGIWGCSGKLICTTAVPWNSSWSNKTQDEIWKNMTWMQWDREINNYTNTIYSLL
EESQNQQEKNEKDLLALDSWKNLWNWFDISNWLW*
Gp140 Clade BC: Accession number, KC492738.
SEQ ID NO: 4
MRVMGIRRNCQHLWRWGIMLLGMLMICSVVGNLWVTVYYGVPVWKEATTTLFCASDAKAY
DTEVHNVWATHACVPTDPNPQEMVLENVTENFNMWKNEMVNQMQEDVISLWDQSLKPCVK
LTPLCVTLKCKNVSSNSTETPKLRGNSSETYKDEEMKNCSFNATTILRDKKQEVYALFYK
LDIAPLLLNSSENSSAYYSLINCNTSAITQACPKVSFDPIPIHYCTPAGYAILKCNDKKF
NGTGPCSNVSTVQCTHGIKPVVSTQLLLNGSLAEGEVIIRSKNLTDNAKTIIVQLNRSVE
IVCTRPNNNTRKSIRIGPGQTFYATGDIIGDIRQAHCNISEDMWNETLHWVSRKLAEHFP
NRTINFTSSSGGDLEIATHSFNCRGEFFYCNTSRLFNGTYMFNGTRGNSSSNSTITIPCR
IKQIINMWQQVGRAMYAPPIEGNLTCRSNITGLLLVRDGGDNTNKTEIFRPQGGDMRDNW
RSELYKYKVVEIKPLGIAPTTAKLTVQARQLLSGIVQQQSNLLRAIEAQQHLLQLTVWGI
KQLQTRVLAIERYLKDQQLLGIWGCSGKLICTTAVPWNSSWSNKTQDEIWNNLTWMQWDK
EISNYTDTIYKLLEDSQNQQERNEKDLLALDSWKNLWSWFDITNWLW*
HIVgag Clade B: Accession number, JF932500.
SEQ ID NO: 5
MGARASVLSGGELDRWEKIRLRPGGKKKYRLKHVVWASRELERFAVNPGLLETSEGCRQI
LEQLQPSLQTGSEELRSLYNTIAVLYCVHQKIEIKDTKEALDKIEEEQNKSKKKAQQAAA
DTGNNSQVSQNYPIVRNLQGQMVHQPLSPRTLNAWVKVVEEKAFSPEVIPMFSALSEGAT
PQDLNTMLNTVGGHQAAMQMLRETINEEAAEWDRLHPPQAGPIAPGQIREPRGSDIAGTT
SNLQEQIAWMTNNPPIPVGEIYKRWIILGLNKIVRMYSPTSILDIKQGPKEPFRDYVDRF
YKTLRAEQASQDVKNWMTETLLVQNANPDCKTILKALGPAATLEEMMTACQGVGGPSHKA
RILAEAMSQVTNSASVMMQRGNFRNQRKPVKCFNCGKEGHIAKNCRAPRKKGCWKCGKEG
HQMKDCTERQANFLGKIWPSHKGRPGNFLQSRPEPTAPPEESFRFGEETTTPSQKQEQID
KELYPLASLKSLFGNDPSSQ*
1, C6 020 CMV-HIVgp140 AE11
SEQ ID NO: 6
catcatcaataatatacctcaaacttttggtgcgcgttaatatgcaaatgagctgtttgaatttg
gggagggaggaaggtgattggctgcgggagcggcgaccgttaggggcggggcgggtgacgttttg
atgacgtggctatgaggcggagccggtttgcaagttctcgtgggaaaagtgacgtcaaacgaggt
gtggtttgaacacggaaatactcaattttcccgcgctctctgacaggaaatgaggtgtttctggg
cggatgcaagtgaaaacgggccattttcgcgcgaaaactgaatgaggaagtgaaaatctgagtaa
tttcgcgtttatggcagggaggagtatttgccgagggccgagtagactttgaccgattacgtggg
ggtttcgattaccgtatttttcacctaaatttccgcgtacggtgtcaaagtccggtgtttttacg
tacgatatcatttccccgaaagtgccacctgaccgtaactataacggtcctaaggtagcgaaagc
tcagatctcccgatcccctatggtgcactctcagtacaatctgctctgatgccgcatagttaagc
cagtatctgctccctgcttgtgtgttggaggtcgctgagtagtgcgcgagcaaaatttaagctac
aacaaggcaaggcttgaccgacaattgcatgaagaatctgcttagggttaggcgttttgcgctgc
ttcgcgatgtacgggccagatatacgcgttgacattgattattgactagttattaatagtaatca
attacggggtcattagttcatagcccatatatggagttccgcgttacataacttacggtaaatgg
cccgcctggctgaccgcccaacgacccccgcccattgacgtcaataatgacgtatgttcccatag
taacgccaatagggactttccattgacgtcaatgggtggagtatttacggtaaactgcccacttg
gcagtacatcaagtgtatcatatgccaagtacgccccctattgacgtcaatgacggtaaatggcc
cgcctggcattatgcccagtacatgaccttatgggactttcctacttggcagtacatctacgtat
tagtcatcgctattaccatggtgatgcggttttggcagtacatcaatgggcgtggatagcggttt
gactcacggggatttccaagtctccaccccattgacgtcaatgggagtttgttttggcaccaaaa
tcaacgggactttccaaaatgtcgtaacaactccgccccattgacgcaaatgggcggtaggcgtg
tacggtgggaggtctatataagcagagctcgtttagtgaaccgtcagatcactagaagctttatt
gcggtagtttatcacagttaaattgctaacgcagtcagtgcttctgacacaacagtctcgaactt
aagctgcagaagttggtcgtgaggcactgggcaggtaagtatcaaggttacaagacaggtttaag
gagaccaatagaaactgggcttgtcgagacagagaagactcttgcgtttctgataggcacctatt
ggtcttactgacatccactttgcctttctctccacaggtgtccactcccagttcaattacagctc
ttaaaaggctagagtacttaatacgactcactataggctagcatgagagtgaaggggacacagat
gaattggccaaacttgtggaaatgggggactttgatccttgggttggtgatcatgtgtagtgcct
cagacaacttgtgggttacagtttattatggagttcctgtgtggagagatgcaaataccacccta
ttttgtgcatcagatgccaaagcacatgagacagaagtgcacaatgtctgggccacatatgcctg
tgtacccacagatcccaacccacaagaaatacccatggaaaatgtgacagaaaattttaacatgt
ggaaaaataacatggtagagcaaatgcaggaggatgtaatcagtttatgggatcaaagtctaaag
ccatgtgtaaagttaactcctctctgcgttactttaatttgtaccaatgctaacttgaccaagat
caacagtaccaatagcgggcctaaagtaataggaaatgtaacagatgaagtaagaaactgttctt
ttaatatgaccacattactaacagataagaagcaaaaggtttatgcacttttttataagcttgat
atagtaccaattgataatagtaatagtagtgagtatagattaataaattgtaatacttcagtcat
taagcaggcttgtccaaagatatcctttgatccaattcctatacattattgtactccagctggtt
atgcgattttaaaatgtaatgataagaatttcaatgggacagggccatgtaaaaatgtcagctca
gtacagtgcacacatggaattaagccagtggtctcaactcaattactgttaaatggcagtctagc
agaagaagagataataatcagatctgaaaatctcacaaacaatgccaaaaccataatagtgcacc
ttaataaggctgtagaaatcaattgtaccagaccctccaacaatacaagaacaagtataagaata
ggaccaggacaaatattttatagaacaggagacataataggagatataagacaagcatattgtga
aattaatggaacaaaatggaatgaaactttaagacaggtagcaaaaaaattaaaagagcaattta
ataacacaataaaattccagccaccctcaggaggagatctagaaattacaatgcttcattttaat
tgtagaggggaatttttctattgcaatacaacaaaactgttcaatagtacttgggaaagaaatga
gaccataaaagggggtaatggcaatggcaatgacactatcatacttccatgcaggataaagcaaa
tcataaacatgtggcaaggagcaggacaagcaatgtatgctcctcccatcagtggaataattaac
tgtgtatcaaatattacaggaatactattgacaagagatggtggtaatactaatgaaactgccga
gatcttcagacctggaggaggaaatataaaggacaattggagaagtgaattatataaatataaag
tagtacaaattgaaccactaggagtagcacccaccaaggcaaagctgacggtacaggccagacaa
ttattgtctggtatagtgcaacagcaaagcaatttgctgagggctatagaggcgcagcagcatat
gttgcaactcacagtctggggcattaaacagctccaggcaagaatcctggctgtggaaagctacc
taaagcatcaacagttcctaggactttggggctgctctaacaaaattatctgcaccactgctgta
ccctggaattcctcttggagtaataaatcttatgatgagatttgggaaaatatgacatggataga
atgggagagagaaattggcaattacacaaaccaaatatatgatatacttacaaaatcgcaggaac
agcaggacaaaaatgaaaaggaactgttggaattggatcaatgggcaagtctgtggaattggttt
agcataacaaaatggctgtggtaatgtacaagtaaagcggccgccactgtgctggatgatccgag
ctcggtacctctagagtcgacccgggcggccaaaccgctgatcagcctcgactgtgccttctagt
tgccagccatctgttgtttgcccctcccccgtgccttccttgaccctggaaggtgccactcccac
tgtcctttcctaataaaatgaggaaattgcatcgcattgtctgagtaggtgtcattctattctgg
ggggtggggtggggcaggacagcaagggggaggattgggaagacaatagcaggcatgctggggat
gcggtgggctctatggcttctgaggcggaaagaaccagcagatctgcagatctgaattcatctat
gtcgggtgcggagaaagaggtaatgaaatggcattatgggtattatgggtctgcattaatgaatc
ggtcagatatcgacatatgctggccaccgtgcatgtggcctcgcacccccgcaagacatggcccg
agttcgagcacaacgtcatgacccgctgcaatgtgcacctgggctcccgccgaggcatgttcatg
ccctaccagtgcaacatgcaatttgtgaaggtgctgctggagcccgatgccatgtccagagtgag
cctgacgggggtgtttgacatgaatgtggagctgtggaaaattctgagatatgatgaatccaaga
ccaggtgccgggcctgcgaatgcggaggcaagcacgccaggcttcagcccgtgtgtgtggaggtg
acggaggacctgcgacccgatcatttggtgttgtcctgcaacgggacggagttcggctccagcgg
ggaagaatctgactagagtgagtagtgtttggggctgggtgtgagcctgcatgaggggcagaatg
actaaaatctgtggttttctgtgtgttgcagcagcatgagcggaagcgcctcctttgagggaggg
gtattcagcccttatctgacggggcgtctcccctcctgggcgggagtgcgtcagaatgtgatggg
atccacggtggacggccggcccgtgcagcccgcgaactcttcaaccctgacctacgcgaccctga
gctcctcgtccgtggacgcagctgccgccgcagctgctgcttccgccgccagcgccgtgcgcgga
atggccctgggcgccggctactacagctctctggtggccaactcgagttccaccaataatcccgc
cagcctgaacgaggagaagctgctgctgctgatggcccagctcgaggccctgacccagcgcctgg
gcgagctgacccagcaggtggctcagctgcaggcggagacgcgggccgcggttgccacggtgaaa
accaaataaaaaatgaatcaataaataaacggagacggttgttgattttaacacagagtcttgaa
tctttatttgatttttcgcgcgcggtaggccctggaccaccggtctcgatcattgagcacccggt
ggatcttttccaggacccggtagaggtgggcttggatgttgaggtacatgggcatgagcccgtcc
cgggggtggaggtagctccattgcagggcctcgtgctcggggatggtgttgtaaatcacccagtc
atagcaggggcgcagggcgtggtgctgcacgatgtccttgaggaggagactgatggccacgggca
gccccttggtgtaggtgttgacgaacctgttgagctgggagggatgcatgcggggggagatgaga
tgcatcttggcctggatcttgagattggcgatgttcccgcccagatcccgccgggggttcatgtt
gtgcaggaccaccagcacggtgtatccggtgcacttggggaatttgtcatgcaacttggaaggga
aggcgtgaaagaatttggagacgcccttgtgaccgcccaggttttccatgcactcatccatgatg
atggcgatgggcccgtgggcggcggcctgggcaaagacgtttcgggggtcggacacatcgtagtt
gtggtcctgggtgagctcgtcataggccattttaatgaatttggggcggagggtgcccgactggg
ggacgaaggtgccctcgatcccgggggcgtagttgccctcgcagatctgcatctcccaggcaagc
aggttccggagcagctgggacttgccgcaaccggtggggccgtagatgaccccgatgaccggctg
caggtggtagttgagggagagacagctgccgtcctcgcggaggaggggggccacctcgttcatca
tctcgcgcacatgcatgttctcgcgcacgagttccgccaggaggcgctcgccccccagcgagagg
agctcttgcagcgaggcgaagtttttcagcggcttgagtccgtcggccatgggcattttggagag
ggtctgttgcaagagttccagacggtcccagagctcggtgatgtgctctagggcatctcgatcca
gcagacctcctcgtttcgcgggttggggcgactgcgggagtagggcaccaggcgatgggcgtcca
gcgaggccagggtccggtccttccagggccgcagggtccgcgtcagcgtggtctccgtcacggtg
aaggggtgcgcgccgggctgggcgcttgcgagggtgcgcttcaggctcatccggctggtcgagaa
ccgctcccggtcggcgccctgcgcgtcggccaggtagcaattgagcatgagttcgtagttgagcg
cctcggccgcgtggcccttggcgcggagcttacctttggaagtgtgtccgcagacgggacagagg
agggacttgagggcgtagagcttgggggcgaggaagacggactcgggggcgtaggcgtccgcgcc
gcagctggcgcagacggtctcgcactccacgagccaggtgaggtcggggcggttggggtcaaaaa
cgaggtttcctccgtgctttttgatgcgtttcttacctctggtctccatgagctcgtgtccccgc
tgggtgacaaagaggctgtccgtgtccccgtagaccgactttatgggccggtcctcgagcggggt
gccgcggtcctcgtcgtagaggaaccccgcccactccgagacgaaggcccgggtccaggccagca
cgaaggaggccacgtgggaggggtagcggtcgttgtccaccagcgggtccaccttctccagggta
tgcaagcacatgtccccctcgtccacatccaggaaggtgattggcttgtaagtgtaggccacgtg
accgggggtcccggccgggggggtataaaagggggcgggcccctgctcgtcctcactgtcttccg
gatcgctgtccaggagcgccagctgttggggtaggtattccctctcgaaggcgggcatgacctcg
gcactcaggttgtcagtttctagaaacgaggaggatttgatattgacggtgccgttggagacgcc
tttcatgagcccctcgtccatttggtcagaaaagacgatctttttgttgtcgagcttggtggcga
aggagccgtagagggcgttggagagcagcttggcgatggagcgcatggtctggttcttttccttg
tcggcgcgctccttggcggcgatgttgagctgcacgtactcgcgcgccacgcacttccattcggg
gaagacggtggtgagctcgtcgggcacgattctgacccgccagccgcggttgtgcagggtgatga
ggtccacgctggtggccacctcgccgcgcaggggctcgttggtccagcagaggcgcccgcccttg
cgcgagcagaaggggggcagcgggtccagcatgagctcgtcgggggggtcggcgtccacggtgaa
gatgccgggcaggagctcggggtcgaagtagctgatgcaggtgcccagattgtccagcgccgctt
gccagtcgcgcacggccagcgcgcgctcgtaggggctgaggggcgtgccccagggcatggggtgc
gtgagcgcggaggcgtacatgccgcagatgtcgtagacgtagaggggctcctcgaggacgccgat
gtaggtggggtagcagcgccccccgcggatgctggcgcgcacgtagtcgtacagctcgtgcgagg
gcgcgaggagccccgtgccgaggttggagcgttgcggcttttcggcgcggtagacgatctggcgg
aagatggcgtgggagttggaggagatggtgggcctttggaagatgttgaagtgggcgtggggcag
gccgaccgagtccctgatgaagtgggcgtaggagtcctgcagcttggcgacgagctcggcggtga
cgaggacgtccagggcgcagtagtcgagggtctcttggatgatgtcatacttgagctggcccttc
tgcttccacagctcgcggttgagaaggaactcttcgcggtccttccagtactcttcgagggggaa
cccgtcctgatcggcacggtaagagcccaccatgtagaactggttgacggccttgtaggcgcagc
agcccttctccacggggagggcgtaagcttgcgcggccttgcgcagggaggtgtgggtgagggcg
aaggtgtcgcgcaccatgaccttgaggaactggtgcttgaagtcgaggtcgtcgcagccgccctg
ctcccagagttggaagtccgtgcgcttcttgtaggcggggttaggcaaagcgaaagtaacatcgt
tgaagaggatcttgcccgcgcggggcatgaagttgcgagtgatgcggaaaggctggggcacctcg
gcccggttgttgatgacctgggcggcgaggacgatctcgtcgaagccgttgatgttgtgcccgac
gatgtagagttccacgaatcgcgggcggcccttgacgtggggcagcttcttgagctcgtcgtagg
tgagctcggcggggtcgctgagcccgtgctgctcgagggcccagtcggcgacgtgggggttggcg
ctgaggaaggaagtccagagatccacggccagggcggtctgcaagcggtcccggtactgacggaa
ctgttggcccacggccattttttcgggggtgacgcagtagaaggtgcgggggtcgccgtgccagc
ggtcccacttgagctggagggcgaggtcgtgggcgagctcgacgagcggcgggtccccggagagt
ttcatgaccagcatgaaggggacgagctgcttgccgaaggaccccatccaggtgtaggtttccac
atcgtaggtgaggaagagcctttcggtgcgaggatgcgagccgatggggaagaactggatctcct
gccaccagttggaggaatggctgttgatgtgatggaagtagaaatgccgacggcgcgccgagcac
tcgtgcttgtgtttatacaagcgtccgcagtgctcgcaacgctgcacgggatgcacgtgctgcac
gagctgtacctgggttcctttggcgaggaatttcagtgggcagtggagcgctggcggctgcatct
cgtgctgtactacgtcttggccatcggcgtggccatcgtctgcctcgatggtggtcatgctgacg
agcccgcgcgggaggcaggtccagacctcggctcggacgggtcggagagcgaggacgagggcgcg
caggccggagctgtccagggtcctgagacgctgcggagtcaggtcagtgggcagcggcggcgcgc
ggttgacttgcaggagcttttccagggcgcgcgggaggtccagatggtacttgatctccacggcg
ccgttggtggctacgtccacggcttgcagggtgccgtgcccctggggcgccaccaccgtgccccg
tttcttcttgggcgctgcttccatgtcggtcagaagcggcggcgaggacgcgcgccgggcggcag
gggcggctcggggcccggaggcaggggcggcaggggcacgtcggcgccgcgcgcgggcaggttct
ggtactgcgcccggagaagactggcgtgagcgacgacgcgacggttgacgtcctggatctgacgc
ctctgggtgaaggccacgggacccgtgagtttgaacctgaaagagagttcgacagaatcaatctc
ggtatcgttgacggcggcctgccgcaggatctcttgcacgtcgcccgagttgtcctggtaggcga
tctcggtcatgaactgctcgatctcctcctcctgaaggtctccgcggccggcgcgctcgacggtg
gccgcgaggtcgttggagatgcggcccatgagctgcgagaaggcgttcatgccggcctcgttcca
gacgcggctgtagaccacggctccgtcggggtcgcgcgcgcgcatgaccacctgggcgaggttga
gctcgacgtggcgcgtgaagaccgcgtagttgcagaggcgctggtagaggtagttgagcgtggtg
gcgatgtgctcggtgacgaagaagtacatgatccagcggcggagcggcatctcgctgacgtcgcc
cagggcttccaagcgttccatggcctcgtagaagtccacggcgaagttgaaaaactgggagttgc
gcgccgagacggtcaactcctcctccagaagacggatgagctcggcgatggtggcgcgcacctcg
cgctcgaaggccccggggggctcctcttccatctcctcctcttcctcctccactaacatctcttc
tacttcctcctcaggaggcggtggcgggggaggggccctgcgtcgccggcggcgcacgggcagac
ggtcgatgaagcgctcgatggtctccccgcgccggcgacgcatggtctcggtgacggcgcgcccg
tcctcgcggggccgcagcatgaagacgccgccgcgcatctccaggtggccgccgggggggtctcc
gttgggcagggagagggcgctgacgatgcatcttatcaattgacccgtagggactccgcgcaagg
acctgagcgtctcgagatccacgggatccgaaaaccgctgaacgaaggcttcgagccagtcgcag
tcgcaaggtaggctgagcccggtttcttgttcttcgggtatttggtcgggaggcgggcgggcgat
gctgctggtgatgaagttgaagtaggcggtcctgagacggcggatggtggcgaggagcaccaggt
ccttgggcccggcttgctggatgcgcagacggtcggccatgccccaggcgtggtcctgacacctg
gcgaggtccttgtagtagtcctgcatgagccgctccacgggcacctcctcctcgcccgcgcggcc
gtgcatgcgcgtgagcccgaacccgcgctgcggctggacgagcgccaggtcggcgacgacgcgct
cggtgaggatggcctgctggatctgggtgagggtggtctggaagtcgtcgaagtcgacgaagcgg
tggtaggctccggtgttgatggtgtaggagcagttggccatgacggaccagttgacggtctggtg
gccgggtcgcacgagctcgtggtacttgaggcgcgagtaggcgcgcgtgtcgaagatgtagtcgt
tgcaggcgcgcacgaggtactggtatccgacgaggaagtgcggcggcggctggcggtagagcggc
catcgctcggtggcgggggcgccgggcgcgaggtcctcgagcatgaggcggtggtagccgtagat
gtacctggacatccaggtgatgccggcggcggtggtggaggcgcgcgggaactcgcggacgcggt
tccagatgttgcgcagcggcaggaagtagttcatggtggccgcggtctggcccgtgaggcgcgcg
cagtcgtggatgctctagacatacgggcaaaaacgaaagcggtcagcggctcgactccgtggcct
ggaggctaagcgaacgggttgggctgcgcgtgtaccccggttcgaatctcgaatcaggctggagc
cgcagctaacgtggtactggcactcccgtctcgacccaagcctgctaacgaaacctccaggatac
ggaggcgggtcgttttttggccttggtcgctggtcatgaaaaactagtaagcgcggaaagcggcc
gcccgcgatggctcgctgccgtagtctggagaaagaatcgccagggttgcgttgcggtgtgcccc
ggttcgagcctcagcgctcggcgccggccggattccgcggctaacgtgggcgtggctgccccgtc
gtttccaagaccccttagccagccgacttctccagttacggagcgagcccctctttttttttctt
gtgtttttgccagatgcatcccgtactgcggcagatgcgcccccaccctccaccacaaccgcccc
taccgcagcagcagcaacagccggcgcttctgcccccgccccagcagcagccagccactaccgcg
gcggccgccgtgagcggagccggcgttcagtatgacctggccttggaagagggcgaggggctggc
gcggctgggggcgtcgtcgccggagcggcacccgcgcgtgcagatgaaaagggacgctcgcgagg
cctacgtgcccaagcagaacctgttcagagacaggagcggcgaggagcccgaggagatgcgcgcc
tcccgcttccacgcggggcgggagctgcggcgcggcctggaccgaaagcgggtgctgagggacga
ggatttcgaggcggacgagctgacggggatcagccccgcgcgcgcgcacgtggccgcggccaacc
tggtcacggcgtacgagcagaccgtgaaggaggagagcaacttccaaaaatccttcaacaaccac
gtgcgcacgctgatcgcgcgcgaggaggtgaccctgggcctgatgcacctgtgggacctgctgga
ggccatcgtgcagaaccccacgagcaagccgctgacggcgcagctgtttctggtggtgcagcaca
gtcgggacaacgagacgttcagggaggcgctgctgaatatcaccgagcccgagggccgctggctc
ctggacctggtgaacattttgcagagcatcgtggtgcaggagcgcgggctgccgctgtccgagaa
gctggcggccatcaacttctcggtgctgagtctgggcaagtactacgctaggaagatctacaaga
ccccgtacgtgcccatagacaaggaggtgaagatcgacgggttttacatgcgcatgaccctgaaa
gtgctgaccctgagcgacgatctgggggtgtaccgcaacgacaggatgcaccgcgcggtgagcgc
cagccgccggcgcgagctgagcgaccaggagctgatgcacagcctgcagcgggccctgaccgggg
ccgggaccgagggggagagctactttgacatgggcgcggacctgcgctggcagcccagccgccgg
gccttggaagctgccggcggttccccctacgtggaggaggtggacgatgaggaggaggagggcga
gtacctggaagactgatggcgcgaccgtatttttgctagatgcagcaacagccaccgccgccgcc
tcctgatcccgcgatgcgggcggcgctgcagagccagccgtccggcattaactcctcggacgatt
ggacccaggccatgcaacgcatcatggcgctgacgacccgcaatcccgaagcctttagacagcag
cctcaggccaaccggctctcggccatcctggaggccgtggtgccctcgcgctcgaaccccacgca
cgagaaggtgctggccatcgtgaacgcgctggtggagaacaaggccatccgcggtgacgaggccg
ggctggtgtacaacgcgctgctggagcgcgtggcccgctacaacagcaccaacgtgcagacgaac
ctggaccgcatggtgaccgacgtgcgcgaggcggtgtcgcagcgcgagcggttccaccgcgagtc
gaacctgggctccatggtggcgctgaacgccttcctgagcacgcagcccgccaacgtgccccggg
gccaggaggactacaccaacttcatcagcgcgctgcggctgatggtggccgaggtgccccagagc
gaggtgtaccagtcggggccggactacttcttccagaccagtcgccagggcttgcagaccgtgaa
cctgagccaggctttcaagaacttgcagggactgtggggcgtgcaggccccggtcggggaccgcg
cgacggtgtcgagcctgctgacgccgaactcgcgcctgctgctgctgctggtggcgcccttcacg
gacagcggcagcgtgagccgcgactcgtacctgggctacctgcttaacctgtaccgcgaggccat
cggacaggcgcacgtggacgagcagacctaccaggagatcacccacgtgagccgcgcgctgggcc
aggaggacccgggcaacctggaggccaccctgaacttcctgctgaccaaccggtcgcagaagatc
ccgccccagtacgcgctgagcaccgaggaggagcgcatcctgcgctacgtgcagcagagcgtggg
gctgttcctgatgcaggagggggccacgcccagcgcggcgctcgacatgaccgcgcgcaacatgg
agcccagcatgtacgcccgcaaccgcccgttcatcaataagctgatggactacttgcatcgggcg
gccgccatgaactcggactactttaccaacgccatcttgaacccgcactggctcccgccgcccgg
gttctacacgggcgagtacgacatgcccgaccccaacgacgggttcctgtgggacgacgtggaca
gcagcgtgttctcgccgcgtccaggaaccaatgccgtgtggaagaaagagggcggggaccggcgg
ccgtcctcggcgctgtccggtcgcgcgggtgctgccgcggcggtgcccgaggccgccagcccctt
cccgagcctgcccttttcgctgaacagcgtgcgcagcagcgagctgggtcggctgacgcgaccgc
gcctgctgggcgaggaggagtacctgaacgactccttgttgaggcccgagcgcgagaagaacttc
cccaataacgggatagagagcctggtggacaagatgagccgctggaagacgtacgcgcacgagca
cagggacgagccccgagctagcagcgcaggcacccgtagacgccagcggcacgacaggcagcggg
gactggtgtgggacgatgaggattccgccgacgacagcagcgtgttggacttgggtgggagtggt
ggtaacccgttcgctcacctgcgcccccgtatcgggcgcctgatgtaagaatctgaaaaaataaa
agacggtactcaccaaggccatggcgaccagcgtgcgttcttctctgttgtttgtagtagtatga
tgaggcgcgtgtacccggagggtcctcctccctcgtacgagagcgtgatgcagcaggcggtggcg
gcggcgatgcagcccccgctggaggcgccttacgtgcccccgcggtacctggcgcctacggaggg
gcggaacagcattcgttactcggagctggcacccttgtacgataccacccggttgtacctggtgg
acaacaagtcggcagacatcgcctcgctgaactaccagaacgaccacagcaacttcctgaccacc
gtggtgcagaacaacgatttcacccccacggaggccagcacccagaccatcaactttgacgagcg
ctcgcggtggggcggccagctgaaaaccatcatgcacaccaacatgcccaacgtgaacgagttca
tgtacagcaacaagttcaaggcgcgggtgatggtctcgcgcaagacccccaacggggtggatgat
gattatgatggtagtcaggacgagctgacctacgagtgggtggagtttgagctgcccgagggcaa
cttctcggtgaccatgaccatcgatctgatgaacaacgccatcatcgacaactacttggcggtgg
ggcggcagaacggggtgctggagagcgacatcggcgtgaagttcgacacgcgcaacttccggctg
ggctgggaccccgtgaccgagctggtgatgccgggcgtgtacaccaacgaggccttccaccccga
catcgtcctgctgcccggctgcggcgtggacttcaccgagagccgcctcagcaacctgctgggca
tccgcaagcggcagcccttccaggagggcttccagatcctgtacgaggacctggaggggggcaac
atccccgcgctcttggatgtcgaagcctacgagaaaagcaaggaggatagcaccgccgcggcgac
cgcagccgtggccaccgcctctaccgaggtgcggggcgataattttgctagcgctgcggcagcgg
ccgaggcggctgaaaccgaaagtaagatagtcatccagccggtggagaaggacagcaaggacagg
agctacaacgtgctcgcggacaagaaaaacaccgcctaccgcagctggtacctggcctacaacta
cggcgaccccgagaagggcgtgcgctcctggacgctgctcaccacctcggacgtcacctgcggcg
tggagcaagtctactggtcgctgcccgacatgatgcaagacccggtcaccttccgctccacgcgt
caagttagcaactacccggtggtgggcgccgagctcctgcccgtctactccaagagcttcttcaa
cgagcaggccgtctactcgcagcagctgcgcgccttcacctcgctcacgcacgtcttcaaccgct
tccccgagaaccagatcctcgtccgcccgcccgcgcccaccattaccaccgtcagtgaaaacgtt
cctgctctcacagatcacgggaccctgccgctgcgcagcagtatccggggagtccagcgcgtgac
cgtcactgacgccagacgccgcacctgcccctacgtctacaaggccctgggcgtcgcccagcaag
atgtacggaggcgctcgccaacgctccacgcaacaccccgtgcgcgtgcgcgggcacttccgcgc
tccctggggcgccctcaagggccgcgtgcgctcgcgcaccaccgtcgacgacgtgatcgaccagg
tggtggccgacgcgcgcaactacacgcccgccgccgcgcccgtctccaccgtggacgccgtcatc
gacagcgtggtggccgacgcgcgccggtacgcccgcaccaagagccggcggcggcgcatcgcccg
gcggcaccggagcacccccgccatgcgcgcggcgcgagccttgctgcgcagggccaggcgcacgg
gacgcagggccatgctcagggcggccagacgcgcggcctccggcagcagcagcgccggcaggacc
cgcagacgcgcggccacggcggcggcggcggccatcgccagcatgtcccgcccgcggcgcggcaa
cgtgtactgggtgcgcgacgccgccaccggtgtgcgcgtgcccgtgcgcacccgcccccctcgca
cttgaagatgctgacttcgcgatgttgatgtgtcccagcggcgaggaggatgtccaagcgcaaat
acaaggaagagatgctccaggtcatcgcgcctgagatctacggccccgcggcggcggtgaaggag
gaaagaaagccccgcaaactgaagcgggtcaaaaaggacaaaaaggaggaggaagatgacggact
ggtggagtttgtgcgcgagttcgccccccggcggcgcgtgcagtggcgcgggcggaaagtgaaac
cggtgctgcggcccggcaccacggtggtcttcacgcccggcgagcgttccggctccgcctccaag
cgctcctacgacgaggtgtacggggacgaggacatcctcgagcaggcggtcgagcgtctgggcga
gtttgcgtacggcaagcgcagccgccccgcgcccttgaaagaggaggcggtgtccatcccgctgg
accacggcaaccccacgccgagcctgaagccggtgaccctgcagcaggtgctaccgagcgcggcg
ccgcgccggggcttcaagcgcgagggcggcgaggatctgtacccgaccatgcagctgatggtgcc
caagcgccagaagctggaggacgtgctggagcacatgaaggtggaccccgaggtgcagcccgagg
tcaaggtgcggcccatcaagcaggtggccccgggcctgggcgtgcagaccgtggacatcaagatc
cccacggagcccatggaaacgcagaccgagcccgtgaagcccagcaccagcaccatggaggtgca
gacggatccctggatgccagcaccagcttccaccagcactcgccgaagacgcaagtacggcgcgg
ccagcctgctgatgcccaactacgcgctgcatccttccatcatccccacgccgggctaccgcggc
acgcgcttctaccgcggctacaccagcagccgccgccgcaagaccaccacccgccgccgtcgtcg
cagccgccgcagcagcaccgcgacttccgccttggtgcggagagtgtatcgcagcgggcgcgagc
ctctgaccctgccgcgcgcgcgctaccacccgagcatcgccatttaactaccgcctcctacttgc
agatatggccctcacatgccgcctccgcgtccccattacgggctaccgaggaagaaagccgcgcc
gtagaaggctgacggggaacgggctgcgtcgccatcaccaccggcggcggcgcgccatcagcaag
cggttggggggaggcttcctgcccgcgctgatccccatcatcgccgcggcgatcggggcgatccc
cggcatagcttccgtggcggtgcaggcctctcagcgccactgagacacaaaaaagcatggatttg
taataaaaaaaaaaatggactgacgctcctggtcctgtgatgtgtgtttttagatggaagacatc
aatttttcgtccctggcaccgcgacacggcacgcggccgtttatgggcacctggagcgacatcgg
caacagccaactgaacgggggcgccttcaattggagcagtctctggagcgggcttaagaatttcg
ggtccacgctcaaaacctatggcaacaaggcgtggaacagcagcacagggcaggcgctgagggaa
aagctgaaagaacagaacttccagcagaaggtggttgatggcctggcctcaggcatcaacggggt
ggttgacctggccaaccaggccgtgcagaaacagatcaacagccgcctggacgcggtcccgcccg
cggggtccgtggagatgccccaggtggaggaggagctgcctcccctggacaagcgcggcgacaag
cgaccgcgtcccgacgcggaggagacgctgctgacgcacacggacgagccgcccccgtacgagga
ggcggtgaaactgggcctgcccaccacgcggcccgtggcgcctctggccaccggagtgctgaaac
ccagcagcagccagcccgcgaccctggacttgcctccgcctcgcccctccacagtggctaagccc
ctgccgccggtggccgtcgcgtcgcgcgccccccgaggccgcccccaggcgaactggcagagcac
tctgaacagcatcgtgggtctgggagtgcagagtgtgaagcgccgccgctgctattaaaagacac
tgtagcgcttaacttgcttgtctgtgtgtatatgtatgtccgccgaccagaaggaggagtgtgaa
gaggcgcgtcgccgagttgcaagatggccaccccatcgatgctgccccagtgggcgtacatgcac
atcgccggacaggacgcttcggagtacctgagtccgggtctggtgcagttcgcccgcgccacaga
cacctacttcagtctggggaacaagtttaggaaccccacggtggcgcccacgcacgatgtgacca
ccgaccgcagccagcggctgacgctgcgcttcgtgcccgtggaccgcgaggacaacacctactcg
tacaaagtgcgctacacgctggccgtgggcgacaaccgcgtgctggacatggccagcacctactt
tgacatccgcggcgtgctggaccggggccctagcttcaaaccctactctggcaccgcctacaaca
gcctagctcccaagggagctcccaattccagccagtgggagcaagcaaaaacaggcaatggggga
actatggaaacacacacatatggtgtggccccaatgggcggagagaatattacaaaagatggtct
tcaaattggaactgacgttacagcgaatcagaataaaccaatttatgccgacaaaacatttcaac
cagaaccgcaagtaggagaagaaaattggcaagaaactgaaaacttttatggcggtagagctctt
aaaaaagacacaaacatgaaaccttgctatggctcctatgctagacccaccaatgaaaaaggagg
tcaagctaaacttaaagttggagatgatggagttccaaccaaagaattcgacatagacctggctt
tctttgatactcccggtggcaccgtgaacggtcaagacgagtataaagcagacattgtcatgtat
accgaaaacacgtatttggaaactccagacacgcatgtggtatacaaaccaggcaaggatgatgc
aagttctgaaattaacctggttcagcagtctatgcccaacagacccaactacattgggttcaggg
acaactttatcggtcttatgtactacaacagcactggcaatatgggtgtgcttgctggtcaggcc
tcccagctgaatgctgtggttgatttgcaagacagaaacaccgagctgtcctaccagctcttgct
tgactctttgggtgacagaacccggtatttcagtatgtggaaccaggcggtggacagttatgacc
ccgatgtgcgcatcatcgaaaaccatggtgtggaggatgaattgccaaactattgcttccccttg
gacggctctggcactaacgccgcataccaaggtgtgaaagtaaaagatggtcaagatggtgatgt
tgagagtgaatgggaaaatgacgatactgttgcagctcgaaatcaattatgtaaaggtaacattt
tcgccatggagattaatctccaggctaacctgtggagaagtttcctctactcgaacgtggccctg
tacctgcccgactcctacaagtacacgccgaccaacgtcacgctgccgaccaacaccaacaccta
cgattacatgaatggcagagtgacacctccctcgctggtagacgcctacctcaacatcggggcgc
gctggtcgctggaccccatggacaacgtcaaccccttcaaccaccaccgcaacgcgggcctgcgc
taccgctccatgctcctgggcaacgggcgctacgtgcccttccacatccaggtgccccaaaagtt
tttcgccatcaagagcctcctgctcctgcccgggtcctacacctacgagtggaacttccgcaagg
acgtcaacatgatcctgcagagctccctaggcaacgacctgcgcacggacggggcctccatcgcc
ttcaccagcatcaacctctacgccaccttcttccccatggcgcacaacaccgcctccacgctcga
ggccatgctgcgcaacgacaccaacgaccagtccttcaacgactacctctcggcggccaacatgc
tctaccccatcccggccaacgccaccaacgtgcccatctccatcccctcgcgcaactgggccgcc
ttccgcggatggtccttcacgcgcctgaagacccgcgagacgccctcgctcggctccgggttcga
cccctacttcgtctactcgggctccatcccctacctagacggcaccttctacctcaaccacacct
tcaagaaggtctccatcaccttcgactcctccgtcagctggcccggcaacgaccgcctcctgacg
cccaacgagttcgaaatcaagcgcaccgtcgacggagagggatacaacgtggcccagtgcaacat
gaccaaggactggttcctggtccagatgctggcccactacaacatcggctaccagggcttctacg
tgcccgagggctacaaggaccgcatgtactccttcttccgcaacttccagcccatgagccgccag
gtcgtggacgaggtcaactacaaggactaccaggccgtcaccctggcctaccagcacaacaactc
gggcttcgtcggctacctcgcgcccaccatgcgccagggccagccctaccccgccaactacccct
acccgctcatcggcaagagcgccgtcgccagcgtcacccagaaaaagttcctctgcgaccgggtc
atgtggcgcatccccttctccagcaacttcatgtccatgggcgcgctcaccgacctcggccagaa
catgctctacgccaactccgcccacgcgctagacatgaatttcgaagtcgaccccatggatgagt
ccacccttctctatgttgtcttcgaagtcttcgacgtcgtccgagtgcaccagccccaccgcggc
gtcatcgaagccgtctacctgcgcacgcccttctcggccggcaacgccaccacctaagccgctct
tgcttcttgcaagatgacggcgggctccggcgagcaggagctcagggccatcctccgcgacctgg
gctgcgggccctgcttcctgggcaccttcgacaagcgcttccctggattcatggccccgcacaag
ctggcctgcgccatcgtgaacacggccggccgcgagaccgggggcgagcactggctggccttcgc
ctggaacccgcgctcccacacatgctacctcttcgaccccttcgggttctcggacgagcgcctca
agcagatctaccagttcgagtacgagggcctgctgcgtcgcagcgccctggccaccgaggaccgc
tgcgtcaccctggaaaagtccacccagaccgtgcagggtccgcgctcggccgcctgcgggctctt
ctgctgcatgttcctgcacgccttcgtgcactggcccgaccgccccatggacaagaaccccacca
tgaacttactgacgggggtgcccaacggcatgctccagtcgccccaggtggaacccaccctgcgc
cgcaaccaggaagcgctctaccgcttcctcaatgcccactccgcctactttcgctcccaccgcgc
gcgcatcgagaaggccaccgccttcgaccgcatgaatcaagacatgtaaaaaaccggtgtgtgta
tgtgaatgctttattcataataaacagcacatgtttatgccaccttctctgaggctctgacttta
tttagaaatcgaaggggttctgccggctctcggcatggcccgcgggcagggatacgttgcggaac
tggtacttgggcagccacttgaactcggggatcagcagcttgggcacggggaggtcggggaacga
gtcgctccacagcttgcgcgtgagttgcagggcgcccagcaggtcgggcgcggagatcttgaaat
cgcagttgggacccgcgttctgcgcgcgagagttgcggtacacggggttgcagcactggaacacc
atcagggccgggtgcttcacgcttgccagcaccgtcgcgtcggtgatgccctccacgtccagatc
ctcggcgttggccatcccgaagggggtcatcttgcaggtctgccgccccatgctgggcacgcagc
cgggcttgtggttgcaatcgcagtgcagggggatcagcatcatctgggcctgctcggagctcatg
cccgggtacatggccttcatgaaagcctccagctggcggaaggcctgctgcgccttgccgccctc
ggtgaagaagaccccgcaggacttgctagagaactggttggtggcgcagccggcgtcgtgcacgc
agcagcgcgcgtcgttgttggccagctgcaccacgctgcgcccccagcggttctgggtgatcttg
gcccggttggggttctccttcagcgcgcgctgcccgttctcgctcgccacatccatctcgatagt
gtgctccttctggatcatcacggtcccgtgcaggcaccgcagcttgccctcggcttcggtgcagc
cgtgcagccacagcgcgcagccggtgcactcccagttcttgtgggcgatctgggagtgcgagtgc
acgaagccctgcaggaagcggcccatcatcgcggtcagggtcttgttgctggtgaaggtcagcgg
gatgccgcggtgctcctcgttcacatacaggtggcagatgcggcggtacacctcgccctgctcgg
gcatcagctggaaggcggacttcaggtcgctctccacgcggtaccggtccatcagcagcgtcatc
acttccatgcccttctcccaggccgaaacgatcggcaggctcagggggttcttcaccgccattgt
catcttagtcgccgccgccgaggtcagggggtcgttctcgtccagggtctcaaacactcgcttgc
cgtccttctcgatgatgcgcacggggggaaagctgaagcccacggccgccagctcctcctcggcc
tgcctttcgtcctcgctgtcctggctgatgtcttgcaaaggcacatgcttggtcttgcggggttt
ctttttgggcggcagaggcggcggcgatgtgctgggagagcgcgagttctcgttcaccacgacta
tttcttcttcttggccgtcgtccgagaccacgcggcggtaggcatgcctcttctggggcagaggc
ggaggcgacgggctctcgcggttcggcgggcggctggcagagccccttccgcgttcgggggtgcg
ctcctggcggcgctgctctgactgacttcctccgcggccggccattgtgttctcctagggagcaa
caacaagcatggagactcagccatcgtcgccaacatcgccatctgcccccgccgccaccgccgac
gagaaccagcagcagaatgaaagcttaaccgccccgccgcccagccccacctccgacgccgcggc
cccagacatgcaagagatggaggaatccatcgagattgacctgggctacgtgacgcccgcggagc
acgaggaggagctggcagcgcgcttttcagccccggaagagaaccaccaagagcagccagagcag
gaagcagagaacgagcagaaccaggctgggcacgagcatggcgactacctgagcggggcagagga
cgtgctcatcaagcatctggcccgccaatgcatcatcgtcaaggacgcgctgctcgaccgcgccg
aggtgcccctcagcgtggcggagctcagccgcgcctacgagcgcaacctcttctcgccgcgcgtg
ccccccaagcgccagcccaacggcacctgtgagcccaacccgcgcctcaacttctacccggtctt
cgcggtgcccgaggccctggccacctaccacctctttttcaagaaccaaaggatccccgtctcct
gccgcgccaaccgcacccgcgccgacgccctgctcaacctgggccccggcgcccgcctacctgat
atcacctccttggaagaggttcccaagatcttcgagggtctgggcagcgacgagactcgggccgc
gaacgctctgcaaggaagcggagaggagcatgagcaccacagcgccctggtggagttggaaggcg
acaacgcgcgcctggcggtcctcaagcgcacggtcgagctgacccacttcgcctacccggcgctc
aacctgccccccaaggtcatgagcgccgtcatggaccaggtgctcatcaagcgcgcctcgcccct
ctcggaggaggagatgcaggaccccgagagttcggacgagggcaagcccgtggtcagcgacgagc
agctggcgcgctggctgggagcgagtagcaccccccagagcctggaagagcggcgcaagctcatg
atggccgtggtcctggtgaccgtggagctggagtgtctgcgccgcttctttgccgacgcggagac
cctgcgcaaggtcgaggagaacctgcactacctcttcaggcacgggttcgtgcgccaggcctgca
agatctccaacgtggagctgaccaacctggtctcctacatgggcatcctgcacgagaaccgcctg
gggcaaaacgtgctgcacaccaccctgcgcggggaggcccgccgcgactacatccgcgactgcgt
ctacctgtacctctgccacacctggcagacgggcatgggcgtgtggcagcagtgcctggaggagc
agaacctgaaagagctctgcaagctcctgcagaagaacctcaaggccctgtggaccgggttcgac
gagcgtaccaccgcctcggacctggccgacctcatcttccccgagcgcctgcggctgacgctgcg
caacgggctgcccgactttatgagccaaagcatgttgcaaaactttcgctctttcatcctcgaac
gctccgggatcctgcccgccacctgctccgcgctgccctcggacttcgtgccgctgaccttccgc
gagtgccccccgccgctctggagccactgctacttgctgcgcctggccaactacctggcctacca
ctcggacgtgatcgaggacgtcagcggcgagggtctgctggagtgccactgccgctgcaacctct
gcacgccgcaccgctccctggcctgcaacccccagctgctgagcgagacccagatcatcggcacc
ttcgagttgcaaggccccggcgacggcgagggcaaggggggtctgaaactcaccccggggctgtg
gacctcggcctacttgcgcaagttcgtgcccgaggactaccatcccttcgagatcaggttctacg
aggaccaatcccagccgcccaaggccgagctgtcggcctgcgtcatcacccagggggccatcctg
gcccaattgcaagccatccagaaatcccgccaagaatttctgctgaaaaagggccacggggtcta
cttggacccccagaccggagaggagctcaaccccagcttcccccaggatgccccgaggaagcagc
aagaagctgaaagtggagctgccgccgccggaggatttggaggaagactgggagagcagtcaggc
agaggaggaggagatggaagactgggacagcactcaggcagaggaggacagcctgcaagacagtc
tggaggaggaagacgaggtggaggaggcagaggaagaagcagccgccgccagaccgtcgtcctcg
gcggagaaagcaagcagcacggataccatctccgctccgggtcggggtcgcggcggccgggccca
cagtaggtgggacgagaccgggcgcttcccgaaccccaccacccagaccggtaagaaggagcggc
agggatacaagtcctggcgggggcacaaaaacgccatcgtctcctgcttgcaagcctgcgggggc
aacatctccttcacccggcgctacctgctcttccaccgcggggtgaacttcccccgcaacatctt
gcattactaccgtcacctccacagcccctactactgtttccaagaagaggcagaaacccagcagc
agcagaaaaccagcggcagcagcagctagaaaatccacagcggcggcaggtggactgaggatcgc
ggcgaacgagccggcgcagacccgggagctgaggaaccggatctttcccaccctctatgccatct
tccagcagagtcgggggcaggagcaggaactgaaagtcaagaaccgttctctgcgctcgctcacc
cgcagttgtctgtatcacaagagcgaagaccaacttcagcgcactctcgaggacgccgaggctct
cttcaacaagtactgcgcgctcactcttaaagagtagcccgcgcccgcccacacacggaaaaagg
cgggaattacgtcaccacctgcgcccttcgcccgaccatcatgagcaaagagattcccacgcctt
acatgtggagctaccagccccagatgggcctggccgccggcgccgcccaggactactccacccgc
atgaactggctcagtgccgggcccgcgatgatctcacgggtgaatgacatccgcgcccaccgaaa
ccagatactcctagaacagtcagcgatcaccgccacgccccgccatcaccttaatccgcgtaatt
ggcccgccgccctggtgtaccaggaaattccccagcccacgaccgtactacttccgcgagacgcc
caggccgaagtccagctgactaactcaggtgtccagctggccggcggcgccgccctgtgtcgtca
ccgccccgctcagggtataaagcggctggtgatccgaggcagaggcacacagctcaacgacgagg
tggtgagctcttcgctgggtctgcgacctgacggagtcttccaactcgccggatcggggagatct
tccttcacgcctcgtcaggccgtcctgactttggagagttcgtcctcgcagccccgctcgggcgg
catcggcactctccagttcgtggaggagttcactccctcggtctacttcaaccccttctccggct
cccccggccactacccggacgagttcatcccgaacttcgacgccatcagcgagtcggtggacggc
tacgattgaatgtcccatggtggcgcagctgacctagctcggcttcgacacctggaccactgccg
ccgcttccgctgcttcgctcgggatctcgccgagtttgcctactttgagctgcccgaggagcacc
ctcagggcccagcccacggagtgcggatcatcgtcgaagggggcctcgactcccacctgcttcgg
atcttcagccagcgaccgatcctggtcgagcgcgaacaaggacagacccttcttactttgtactg
catctgcaaccaccccggcctgcatgaaagtctttgttgtctgctgtgtactgagtataataaaa
gctgagatcagcgactactccggactcgattgtggtgttcctgctatcaaccggtccctgttctt
caccgggaacgagaccgagctccagctccagtgtaagccccacaagaagtacctcacctggctgt
tccagggctccccgatcgccgttgtcaaccactgcgacaacgacggagtcctgctgagcggccct
gccaaccttactttttccacccgcagaagcaagctccagctcttccaacccttcctccccgggac
ctatcagtgcgtctcaggaccctgccatcacaccttccacctgatcccgaataccacagcgccgc
tccccgctactaacaaccaaactacccaccaacgccaccgtcgcgacctttcctctgaatctaat
accactaccggaggtggcttctgctgttagtgctcccccgtcccgtcgacccccggtcccccact
cagtcccccgaggaggttcgcaaatgcaaattccaagaaccctggaaattcctcaaatgctaccg
ccaaaaatcagacatgcatcccagctggatcatgatcattgggatcgtgaacattctggcctgca
ccctcatctcctttgtgatttacccctgctttgactttggttggaactcgccagaggcgctctat
ctcccgcctgaacctgacacaccaccacagcagcaacctcaggcacacgcactaccaccaccaca
gcctaggccacaatacatgcccatattagactatgaggccgagccacagcgacccatgctccccg
ctattagttacttcaatctaaccggcggagatgactgacccactggccaataacaacgtcaacga
ccttctcctggacatggacggccgcgcctcggagcagcgactcgcccaacttcgcattcgtcagc
agcaggagagagccgtcaaggagctgcaggacggcatagccatccaccagtgcaagagaggcatc
ttctgcctggtgaaacaggccaagatctcctacgaggtcacccagaccgaccatcgcctctccta
cgagctcctgcagcagcgccagaagttcacctgcctggtcggagtcaaccccatcgtcatcaccc
agcagtcgggcgataccaaggggtgcatccactgctcctgcgactcccccgactgcgtccacact
ctgatcaagaccctctgcggcctccgcgacctcctccccatgaactaatcacccccttatccagt
gaaataaagatcatattgatgatgatttaaataaaaaaaataatcatttgatttgaaataaagat
acaatcatattgatgatttgagtttaacaaaaataaagaatcacttacttgaaatctgataccag
gtctctgtccatgttttctgccaacaccacctcactcccctcttcccagctctggtactgcaggc
cccggcgggctgcaaacttcctccacacgctgaaggggatgtcaaattcctcctgtccctcaatc
ttcattttatcttctatcagatgtccaaaaagcgcgtccgggtggatgatgacttcgaccccgtc
tacccctacgatgcagacaacgcaccgaccgtgcccttcatcaacccccccttcgtctcttcaga
tggattccaagagaagcccctgggggtgttgtccctgcgactggctgaccccgtcaccaccaaga
acggggaaatcaccctcaagctgggagagggggtggacctcgactcgtcgggaaaactcatctcc
aacacggccaccaaggccgccgcccctctcagtatttcaaacaacaccatttcccttaaaactgc
tgcccctttctacaacaacaatggaactttaagcctcaatgtctccacaccattagcagtatttc
ccacatttaacactttaggcataagtcttggaaacggtcttcagacttcaaataagttgttgact
gtacaactaactcatcctcttacattcagctcaaatagcatcacagtaaaaacagacaaagggct
atatattaactccagtggaaacagaggacttgaggctaatataagcctaaaaagaggactagttt
ttgacggtaatgctattgcaacatatattggaaatggcttagactatggatcttatgatagtgat
ggaaaaacaagacccgtaattaccaaaattggagcaggattaaattttgatgctaacaaagcaat
agctgtcaaactaggcacaggtttaagttttgactccgctggtgccttgacagctggaaacaaac
aggatgacaagctaacactttggactacccctgacccaagccctaattgtcaattactttcagac
agagatgccaaatttactctctgtcttacaaaatgcggtagtcaaatactaggcactgtggcagt
ggcggctgttactgtaggatcagcactaaatccaattaatgacacagtcaaaagcgccatagttt
tccttagatttgattccgatggtgtactcatgtcaaactcatcaatggtaggtgattactggaac
tttagggagggacagaccactcaaagtgtagcctatacaaatgctgtgggattcatgccaaatat
aggtgcatatccaaaaacccaaagtaaaacacctaaaaatagcatagtcagtcaggtatatttaa
ctggagaaactactatgccaatgacactaaccataactttcaatggcactgatgaaaaagacaca
accccagttagcacctactctatgacttttacatggcagtggactggagactataaggacaaaaa
tattacctttgctaccaactcattctctttttcctacatcgcccaggaataatcccacccagcaa
gccaaccccttttcccaccacctttgtctatatggaaactctgaaacagaaaaataaagttcaag
tgttttattgaatcaacagttttacaggactcgagcagttatttttcctccaccctcccaggaca
tggaatacaccaccctctccccccgcacagccttgaacatctgaatgccattggtgatggacatg
cttttggtctccacgttccacacagtttcagagcgagccagtctcggatcggtcagggagatgaa
accctccgggcactcccgcatctgcacctcacagctcaacagctgaggattgtcctcggtggtcg
ggatcacggttatctggaagaagcagaagagcggcggtgggaatcatagtccgcgaacgggatcg
gccggtggIgtcgcatcaggccccgcagcagtcgctgccgccgccgctccgtcaagctgctgctcagggggttc
gggtccagggactccctcagcatgatgcccacggccctcagcatcagtcgtctggtgcggcgggc
gcagcagcgcatgcgaatctcgctcaggtcactgcagtacgtgcaacacaggaccaccaggttgt
tcaacagtccatagttcaacacgctccagccgaaactcatcgcgggaaggatgctacccacgtgg
ccgtcgtaccagatcctcaggtaaatcaagtggcgctccctccagaagacgctgcccatgtacat
gatctccttgggcatgtggcggttcaccacctcccggtaccacatcaccctctggttgaacatgc
agccccggatgatcctgcggaaccacagggccagcaccgccccgcccgccatgcagcgaagagac
cccggatcccggcaatgacaatggaggacccaccgctcgtacccgtggatcatctgggagctgaa
caagtctatgttggcacagcacaggcatatgctcatgcatctcttcagcactctcagctcctcgg
gggtcaaaaccatatcccagggcacggggaactcttgcaggacagcgaaccccgcagaacagggc
aatcctcgcacataacttacattgtgcatggacagggtatcgcaatcaggcagcaccgggtgatc
ctccaccagagaagcgcgggtctcggtctcctcacagcgtggtaagggggccggccgatacgggt
gatggcgggacgcggctgatcgtgttctcgaccgtgtcatgatgcagttgctttcggacattttc
gtacttgctgtagcagaacctggtccgggcgctgcacaccgatcgccggcggcggtctcggcgct
tggaacgctcggtgttaaagttgtaaaacagccactctctcagaccgtgcagcagatctagggcc
tcaggagtgatgaagatcccatcatgcctgatagctctgatcacatcgaccaccgtggaatgggc
caggcccagccagatgatgcaattttgttgggtttcggtgacggcgggggagggaagaacaggaa
gaaccatgattaacttttaatccaaacggtctcggagcacttcaaaatgaaggtcacggagatgg
cacctctcgcccccgctgtgttggtggaaaataacagccaggtcaaaggtgatacggttctcgag
atgttccacggtggcttccagcaaagcctccacgcgcacatccagaaacaagacaatagcgaaag
cgggagggttctctaattcctcaaccatcatgttacactcctgcaccatccccagataattttca
tttttccagccttgaatgattcgaactagttcctgaggtaaatccaagccagccatgataaaaag
ctcgcgcagagcaccctccaccggcattcttaagcacaccctcataattccaagatattctgctc
ctggttcacctgcagcagattgacaagcggaatatcaaaatctctgccgcgatccctgagctcct
ccctcagcaataactgtaagtactctttcatatcgtctccgaaatttttagccataggaccccca
ggaataagagaagggcaagccacattacagataaaccgaagtcccccccagtgagcattgccaaa
tgtaagattgaaataagcatgctggctagacccggtgatatcttccagataactggacagaaaat
cgggtaagcaatttttaagaaaatcaacaaaagaaaaatcttccaggtgcacgtttagggcctcg
ggaacaacgatggagtaagtgcaagggcgttctctccagcaccaggcaggccacggggtctccgg
cgcgaccctcgtaaaaattgtcgctatgattgaaaaccatcacagagagacgttcccggtggccg
gcgtgaatgattcgagaagaagcatacacccccggaacattggagtccgtgagtgaaaaaaagcg
gccgaggaagcaatgaggcactacaacgctcactctcaagtccagcaaagcgatgccatgcggat
gaagcacaaaattttcaggtgcgtaaaaaatgtaattactcccctcctgcacaggcagcgaagct
cccgatccctccagatacacatacaaagcctcagcgtccatagcttaccgagcggcagcagcagc
ggcacacaacaggcgcaagagtcagagaaaagactgagctctaacctgtccgcccgctctctgct
caatatatagccccagatctacactgacgtaaaggccaaagtctaaaaatacccgccaaataatc
acacacgcccagcacacgcccagaaaccggtgacacactcagaaaaatacgcgcacttcctcaaa
cggccaaactgccgtcatttccgggttcccacgctacgtcatcaaaacacgactttcaaattccg
tcgaccgttaaaaacatcacccgccccgcccctaacggtcgccgctcccgcagccaatcaccttc
ctccctccccaaattcaaacagctcatttgcatattaacgcgcaccaaaagtttgaggtatatta
Ttgatgatg
2, C7010CMV-HIVgp140 AE1.
SEQ ID NO: 7
catcatcaataatatacctcaaacttttggtgcgcgttaatatgcaaatgagctgtttgaatttg
gggagggaggaaggtgattggccgagagacgggcgaccgttaggggcggggcgggtgacgttttg
atgacgtggccgtgaggcggagccggtttgcaagttctcgtgggaaaagtgacgtcaaacgaggt
gtggtttgaacacggaaatactcaattttcccgcgctctctgacaggaaatgaggtgtttctggg
cggatgcaagtgaaaacgggccattttcgcgcgaaaactgaatgaggaagtgaaaatctgagtaa
tttcgcgtttatggcagggaggagtatttgccgagggccgagtagactttgaccgattacgtggg
ggtttcgattaccgtatttttcacctaaatttccgcgtacggtgtcaaagtccggtgtttttacg
tacgatatcatttccccgaaagtgccacctgaccgtaactataacggtcctaaggtagcgaaagc
tcagatctcccgatcccctatggtgcactctcagtacaatctgctctgatgccgcatagttaagc
cagtatctgctccctgcttgtgtgttggaggtcgctgagtagtgcgcgagcaaaatttaagctac
aacaaggcaaggcttgaccgacaattgcatgaagaatctgcttagggttaggcgttttgcgctgc
ttcgcgatgtacgggccagatatacgcgttgacattgattattgactagttattaatagtaatca
attacggggtcattagttcatagcccatatatggagttccgcgttacataacttacggtaaatgg
cccgcctggctgaccgcccaacgacccccgcccattgacgtcaataatgacgtatgttcccatag
taacgccaatagggactttccattgacgtcaatgggtggagtatttacggtaaactgcccacttg
gcagtacatcaagtgtatcatatgccaagtacgccccctattgacgtcaatgacggtaaatggcc
cgcctggcattatgcccagtacatgaccttatgggactttcctacttggcagtacatctacgtat
tagtcatcgctattaccatggtgatgcggttttggcagtacatcaatgggcgtggatagcggttt
gactcacggggatttccaagtctccaccccattgacgtcaatgggagtttgttttggcaccaaaa
tcaacgggactttccaaaatgtcgtaacaactccgccccattgacgcaaatgggcggtaggcgtg
tacggtgggaggtctatataagcagagctcgtttagtgaaccgtcagatcactagaagctttatt
gcggtagtttatcacagttaaattgctaacgcagtcagtgcttctgacacaacagtctcgaactt
aagctgcagaagttggtcgtgaggcactgggcaggtaagtatcaaggttacaagacaggtttaag
gagaccaatagaaactgggcttgtcgagacagagaagactcttgcgtttctgataggcacctatt
ggtcttactgacatccactttgcctttctctccacaggtgtccactcccagttcaattacagctc
ttaaaaggctagagtacttaatacgactcactataggctagcatgagagtgaaggggacacagat
gaattggccaaacttgtggaaatgggggactttgatccttgggttggtgatcatgtgtagtgcct
cagacaacttgtgggttacagtttattatggagttcctgtgtggagagatgcaaataccacccta
ttttgtgcatcagatgccaaagcacatgagacagaagtgcacaatgtctgggccacatatgcctg
tgtacccacagatcccaacccacaagaaatacccatggaaaatgtgacagaaaattttaacatgt
ggaaaaataacatggtagagcaaatgcaggaggatgtaatcagtttatgggatcaaagtctaaag
ccatgtgtaaagttaactcctctctgcgttactttaatttgtaccaatgctaacttgaccaagat
caacagtaccaatagcgggcctaaagtaataggaaatgtaacagatgaagtaagaaactgttctt
ttaatatgaccacattactaacagataagaagcaaaaggtttatgcacttttttataagcttgat
atagtaccaattgataatagtaatagtagtgagtatagattaataaattgtaatacttcagtcat
taagcaggcttgtccaaagatatcctttgatccaattcctatacattattgtactccagctggtt
atgcgattttaaaatgtaatgataagaatttcaatgggacagggccatgtaaaaatgtcagctca
gtacagtgcacacatggaattaagccagtggtctcaactcaattactgttaaatggcagtctagc
agaagaagagataataatcagatctgaaaatctcacaaacaatgccaaaaccataatagtgcacc
ttaataaggctgtagaaatcaattgtaccagaccctccaacaatacaagaacaagtataagaata
ggaccaggacaaatattttatagaacaggagacataataggagatataagacaagcatattgtga
aattaatggaacaaaatggaatgaaactttaagacaggtagcaaaaaaattaaaagagcaattta
ataacacaataaaattccagccaccctcaggaggagatctagaaattacaatgcttcattttaat
tgtagaggggaatttttctattgcaatacaacaaaactgttcaatagtacttgggaaagaaatga
gaccataaaagggggtaatggcaatggcaatgacactatcatacttccatgcaggataaagcaaa
tcataaacatgtggcaaggagcaggacaagcaatgtatgctcctcccatcagtggaataattaac
tgtgtatcaaatattacaggaatactattgacaagagatggtggtaatactaatgaaactgccga
gatcttcagacctggaggaggaaatataaaggacaattggagaagtgaattatataaatataaag
tagtacaaattgaaccactaggagtagcacccaccaaggcaaagctgacggtacaggccagacaa
ttattgtctggtatagtgcaacagcaaagcaatttgctgagggctatagaggcgcagcagcatat
gttgcaactcacagtctggggcattaaacagctccaggcaagaatcctggctgtggaaagctacc
taaagcatcaacagttcctaggactttggggctgctctaacaaaattatctgcaccactgctgta
ccctggaattcctcttggagtaataaatcttatgatgagatttgggaaaatatgacatggataga
atgggagagagaaattggcaattacacaaaccaaatatatgatatacttacaaaatcgcaggaac
agcaggacaaaaatgaaaaggaactgttggaattggatcaatgggcaagtctgtggaattggttt
agcataacaaaatggctgtggtaatgtacaagtaaagcggccgccactgtgctggatgatccgag
ctcggtacctctagagtcgacccgggcggccaaaccgctgatcagcctcgactgtgccttctagt
tgccagccatctgttgtttgcccctcccccgtgccttccttgaccctggaaggtgccactcccac
tgtcctttcctaataaaatgaggaaattgcatcgcattgtctgagtaggtgtcattctattctgg
ggggtggggtggggcaggacagcaagggggaggattgggaagacaatagcaggcatgctggggat
gcggtgggctctatggcttctgaggcggaaagaaccagcagatctgcagatctgaattcatctat
gtcgggtgcggagaaagaggtaatgaaatggcattatgggtattatgggtctgcattaatgaatc
ggccagatatcgatatgctggccaccgtgcatgtgacctcgcacccccgcaagacatggcccgag
ttcgagcacaacgtcatgacccgatgcaatgtgcacctggggtcccgccgaggcatgttcatgcc
ctaccagtgcaacatgcaatttgtgaaggtgctgctggagcccgatgccatgtccagagtgagcc
tgacgggggtgtttgacatgaatgtggagctgtggaaaattctgagatatgatgaatccaagacc
aggtgccgggcctgcgaatgcggaggcaagcacgccaggcttcagcccgtgtgtgtggaggtgac
ggaggacctgcgacccgatcatttggtgttgtcctgcaacgggacggagttcggctccagcgggg
aagaatctgactagagtgagtagtgtttgggggaggtggagggcttgtatgaggggcagaatgac
taaaatctgtgtttttctgtgtgttgcagcagcatgagcggaagcgcctcctttgagggaggggt
attcagcccttatctgacggggcgtctcccctcctgggcgggagtgcgtcagaatgtgatgggat
ccacggtggacggccggcccgtgcagcccgcgaactcttcaaccctgacctacgcgaccctgagc
tcctcgtccgtggacgcagctgccgccgcagctgctgcttccgccgccagcgccgtgcgcggaat
ggccctgggcgccggctactacagctctctggtggccaactcgacttccaccaataatcccgcca
gcctgaacgaggagaagctgctgctgctgatggcccagctcgaggccctgacccagcgcctgggc
gagctgacccagcaggtggctcagctgcaggcggagacgcgggccgcggttgccacggtgaaaac
caaataaaaaatgaatcaataaataaacggagacggttgttgattttaacacagagtcttgaatc
tttatttgatttttcgcgcgcggtaggccctggaccaccggtctcgatcattgagcacccggtgg
attttttccaggacccggtagaggtgggcttggatgttgaggtacatgggcatgagcccgtcccg
ggggtggaggtagctccattgcagggcctcgtgctcgggggtggtgttgtaaatcacccagtcat
agcaggggcgcagggcgtggtgctgcacgatgtccttgaggaggagactgatggccacgggcagc
cccttggtgtaggtgttgacgaacctgttgagctgggagggatgcatgcggggggagatgagatg
catcttggcctggatcttgagattggcgatgttcccgcccagatcccgccgggggttcatgttgt
gcaggaccaccagcacggtgtatccggcgcacttggggaatttgtcatgcaacttggaagggaag
gcgtgaaagaatttggagacgcccttgtgaccgcccaggttttccatgcactcatccatgatgat
ggcgatgggcccgtgggcggcggcctgggcaaagacgtttcgggggtcggacacatcgtagttgt
ggtcctgggtgagctcgtcataggccattttaatgaatttggggcggagggtgcccgactggggg
acgaaggtgccctcgatcccgggggcgtagttgccctcgcagatctgcatctcccaggccttgag
ctcggagggggggatcatgtccacctgcggggcgatgaaaaaaacggtttccggggcgggggaga
tgagctgggccgaaagcaggttccggagcagctgggacttgccgcagccggtggggccgtagatg
accccgatgaccggctgcaggtggtagttgagggagagacagctgccgtcctcgcggaggagggg
ggccacctcgttcatcatctcgcgcacatgcatgttctcgcgcacgagttccgccaggaggcgct
cgccccccagcgagaggagctcttgcagcgaggcgaagtttttcagcggcttgagyccgtcggcc
atgggcattttggagagggtctgttgcaagagttccagacggtcccagagctcggtgatgtgctc
tagggcatctcgatccagcagacctcctcgtttcgcgggttggggcgactgcgggagtagggcac
caggcgatgggcgtccagcgaggccagggtccggtccttccagggtcgcagggtccgcgtcagcg
tggtctccgtcacggtgaaggggtgcgcgccgggctgggcgcttgcgagggtgcgcttcaggctc
atccggctggtcgagaaccgctcccggtcggcgccctgcgcgtcggccaggtagcaattgagcat
gagttcgtagttgagcgcctcggccgcgtggcccttggcgcggagcttacctttggaagtgtgtc
cgcagacgggacagaggagggacttgagggcgtagagcttgggggcgaggaagacggactcgggg
gcgtaggcgtccgcgccgcagctggcgcagacggtctcgcactccacgagccaggtgaggtcggg
ccggttggggtcaaaaacgaggtttcctccgtgctttttgatgcgtttcttacctctggtctcca
tgagctcgtgtccccgctgggtgacaaagaggctgtccgtgtccccgtagaccgactttatgggc
cggtcctcgagcggggtgccgcggtcctcgtcgtagaggaaccccgcccactccgagacgaaggc
ccgggtccaggccagcacgaaggaggccacgtgggaggggtagcggtcgttgtccaccagcgggt
ccaccttctccagggtatgcaagcacatgtccccctcgtccacatccaggaaggtgattggcttg
taagtgtaggccacgtgaccgggggtcccggccgggggggtataaaagggggcgggcccctgctc
gtcctcactgtcttccggatcgctgtccaggagcgccagctgttggggtaggtattccctctcga
aggctggcataacctcggcactcaggttgtcagtttctagaaacgaggaggatttgatattgacg
gtgccgttggagacgcctttcatgagcccctcgtccatctggtcagaaaagacgatctttttgtt
gtcgagcttggtggcgaaggagccgtagagggcgttggagaggagcttggcgatggagcgcatgg
tctggttcttttccttgtcggcgcgctccttggcggcgatgttgagctgcacgtactcgcgcgcc
acgcacttccattcggggaagacggtggtgagctcgtcgggcacgattctgacccgccagccgcg
gttgtgcagggtgatgaggtccacgctggtggccacctcgccgcgcaggggctcgttggtccagc
agaggcgcccgcccttgcgcgagcagaaggggggcagcgggtccagcatgagctcgtcggggggg
tcggcgtccacggtgaagatgccgggcagaagctcggggtcgaagtagctgatgcaggtgtccag
atcgtccagcgccgcttgccagtcgcgcacggccagcgcgcgctcgtaggggctgaggggcgtgc
cccagggcatggggtgcgtgagcgcggaggcgtacatgccgcagatgtcgtagacgtagaggggc
tcctcgaggacgccgatgtaggtggggtagcagcgccccccgcggatgctggcgcgcacgtagtc
gtacagctcgtgcgagggcgcgaggagccccgtgccgaggttggagcgttgcggcttttcggcgc
ggtagacgatctggcggaagatggcgtgggagttggaggagatggtgggcctctggaagatgttg
aagtgggcgtggggcaggccgaccgagtccctgatgaagtgggcgtaggagtcctgcagcttggc
gacgagctcggcggtgacgaggacgtccagggcgcagtagtcgagggtctcttggatgatgtcgt
acttgagctggcccttctgcttccacagctcgcggttgagaaggaactcttcgcggtccttccag
tactcttcgagggggaacccgtcctgatcggcacggtaagagcccaccatgtagaactggttgac
ggccttgtaggcgcagcagcccttctccacggggagggcgtaagcttgtgcggccttgcgcaggg
aggtgtgggtgagggcgaaggtgtcgcgcaccatgaccttgaggaactggtgcttgaagtcgagg
tcgtcgcagccgccctgctcccagagctggaagtccgtgcgcttcttgtaggcggggttgggcaa
agcgaaagtaacatcgttgaagaggatcttgcccgcgcggggcatgaagttgcgagtgatgcgga
aaggctggggcacctcggcccggttgttgatgacctgggcggcgaggacgatctcgtcgaagccg
ttgatgttgtgcccgacgatgtagagttccacgaatcgcgggcggcccttaacgtggggcagctt
cttgagctcgtcgtaggtgagctcggcggggtcgctgagcccgtgctgctcgagggcccagtcgg
cgacgtgggggttggcgctgaggaaggaagtccagagatccacggccagggcggtctgcaagcgg
tcccggtactgacggaactgctggcccacggccattttttcgggggtgacgcagtagaaggtgcg
ggggtcgccgtgccagcggtcccacttgagctggagggcgaggtcgtgggcgagctcgacgagcg
gcgggtccccggagagtttcatgaccagcatgaaggggacgagctgcttgccgaaggaccccatc
caggtgtaggtttccacatcgtaggtgaggaagagcctttcggtgcgaggatgcgagccgatggg
gaagaactggatctcctgccaccagttggaggaatggctgttgatgtgatggaagtagaaatgcc
gacggcgcgccgagcactcgtgcttgtgtttatacaagcgtccgcagtgctcgcaacgctgcacg
ggatgcacgtgctgcacgagctgtacctgggttcctttgacgaggaatttcagtgggcagtggag
cgctggcggctgcatctggtgctgtactacgtcctggccatcggcgtggccatcgtctgcctcga
tggtggtcatgctgacgagcccgcgcgggaggcaggtccagacttcggctcggacgggtcggaga
gcgaggacgagggcgcgcaggccggagctgtccagggtcctgagacgctgcggagtcaggtcagt
gggcagcggcggcgcgcggttgacttgcaggagcttttccagggcgcgcgggaggtccagatggt
acttgatctccacggcgccgttggtggcgacgtccacggcttgcagggtcccgtgcccctggggc
gccaccaccgtgccccgtttcttcttgggcgctgcttccatgccggtcagaagcggcggcgagga
cgcgcgccgggcggcaggggcggctcgggacccggaggcaggggcggcaggggcacgtcggcgcc
gcgcgcgggcaggttctggtactgcgcccggagaagactggcgtgagcgacgacgcgacggttga
cgtcctggatctgacgcctctgggtgaaggccacgggacccgtgagtttgaacctgaaagagagt
tcgacagaatcaatctcggtatcgttgacggcggcctgccgcaggatctcttgcacgtcgcccga
gttgtcctggtaggcgatctcggtcatgaactgctcgatctcctcctcctgaaggtctccgcggc
cggcgcgctcgacggtggccgcgaggtcgttggagatgcggcccatgagctgcgagaaggcgttc
atgccggcctcgttccagacgcggctgtagaccacggctccgtcggggtcgcgcgcgcgcatgac
cacctgggcgaggttgagctcgacgtggcgcgtgaagaccgcgtagttgcagaggcgctggtaga
ggtagttgagcgtggtggcgatgtgctcggtgacgaagaagtacatgatccagcggcggagcggc
atctcgctgacgtcgcccagggcttccaagcgctccatggcctcgtagaagtccacggcgaagtt
gaaaaactgggagttgcgcgccgagacggtcaactcctcctccagaagacggatgagctcagcga
tggtggcgcgcacctcgcgctcgaaggccccggggggctcctcttcttccatctcttcctcctcc
actaacatctcttctacttcctcctcaggaggcggcggcgggggaggggccctgcgtcgccggcg
gcgcacgggcagacggtcgatgaagcgctcgatggtctccccgcgccggcgacgcatggtctcgg
tgacggcgcgcccgtcctcgcggggccgcagcgtgaagacgccgccgcgcatctccaggtggccg
ccgggggggtctccgttgggcagggagagggcgctgacgatgcatcttatcaattggcccgtagg
gactccgcgcaaggacctgagcgtctcgagatccacgggatccgaaaaccgctgaacgaaggctt
cgagccagtcgcagtcgcaaggtaggctgagcccggtttcttgttcttcggggatttcgggaggc
gggcgggcgatgctgctggtgatgaagttgaagtaggcggtcctgagacggcggatggtggcgag
gagcaccaggtccttgggcccggcttgctggatgcgcagacggtcggccatgccccaggcgtggt
cctgacacctggcgaggtccttgtagtagtcctgcatgagccgctccacgggcacctcctcctcg
cccgcgcggccgtgcatgcgcgtgagcccgaacccgcgctggggctggacgagcgccaggtcggc
gacgacgcgctcggcgaggatggcctgctgtatctgggtgagggtggtctggaagtcgtcgaagt
cgacgaagcggtggtaggctccggtgttgatggtataggagcagttggccatgacggaccagttg
acggtctggtggccgggtcgcacgagctcgtggtacttgaggcgcgagtaggcgcgcgtgtcgaa
gatgtagtcgttgcaggtgcgcacgaggtactggtatccgacgaggaagtgcggcggcggctggc
ggtagagcggccatcgctcggtggcgggggcgccgggcgcgaggtcctcgagcatgaggcggtgg
tagccgtagatgtacctggacatccaggtgatgccggcggcggtggtggaggcgcgcgggaactc
gcggacgcggttccagatgttgcgcagcggcaggaagtagttcatggtggccgcggtctggcccg
tgaggcgcgcgcagtcgtggatgctctagacatacgggcaaaaacgaaagcggtcagcggctcga
ctccgtggcctggaggctaagcgaacgggttgggctgcgcgtgtaccccggttcgaatctcgaat
caggctggagccgcagctaacgtggtactggcactcccgtctcgacccaagcctgctaacgaaac
ctccaggatacggaggcgggtcgttttttggccttggtcgctggtcatgaaaaactagtaagcgc
ggaaagcgaccgcccgcgatggctcgctgccgtagtctggagaaagaatcgccagggttgcgttg
cggtgtgccccggttcgagcctcagcgctcggcgccggccggattccgcggctaacgtgggcgtg
gctgccccgtcgtttccaagaccccttagccagccgacttctccagttacggagcgagcccctct
ttttcttgtgtttttgccagatgcatcccgtactgcggcagatgcgcccccaccctccacctcaa
ccgcccctaccgccgcagcagcagcaacagccggcgcttctgcccccgccccagcagcagccagc
cactaccgcggcggccgccgtgagcggagccggcgttcagtatgacctggccttggaagagggcg
aggggctggcgcggctgggggcgtcgtcgccggagcggcacccgcgcgtgcagatgaaaagggac
gctcgcgaggcctacgtgcccaagcagaacctgttcagagacaggagcggcgaggagcccgagga
gatgcgcgcctcccgcttccacgcggggcgggagctgcggcgcggcctggaccgaaagcgggtgc
tgagggacgaggatttcgaggcggacgagctgacggggatcagccccgcgcgcgcgcacgtggcc
gcggccaacctggtcacggcgtacgagcagaccgtgaaggaggagagcaacttccaaaaatcctt
caacaaccacgtgcgcacgctgatcgcgcgcgaggaggtgaccctgggcctgatgcacctgtggg
acctgctggaggccatcgtgcagaaccccacgagcaagccgctgacggcgcagctgtttctggtg
gtgcagcacagtcgggacaacgagacgttcagggaggcgctgctgaatatcaccgagcccgaggg
ccgctggctcctggacctggtgaacattctgcagagcatcgtggtgcaggagcgcgggctgccgc
tgtccgagaagctggcggctatcaacttctcggtgctgagcctgggcaagtactacgctaggaag
atctacaagaccccgtacgtgcccatagacaaggaggtgaagatcgacgggttttacatgcgcat
gaccctgaaagtgctgaccctgagcgacgatctgggggtgtaccgcaacgacaggatgcaccgcg
cggtgagcgccagccgccggcgcgagctgagcgaccaggagctgatgcacagcctgcagcgggcc
ctgaccggggccgggaccgagggggagagctactttgacatgggcgcggacctgcgctggcagcc
cagccgccgggccttggaagctgccggcggttccccctacgtggaggaggtggacgatgaggagg
aggagggcgagtacctggaagactgatggcgcgaccgtatttttgctagatgcagcaacagccac
cgcctcctgatcccgcgatgcgggcggcgctgcagagccagccgtccggcattaactcctcggac
gattggacccaggccatgcaacgcatcatggcgctgacgacccgcaatcccgaagcctttagaca
gcagcctcaggccaaccggctctcggccatcctggaggccgtggtgccctcgcgctcgaacccca
cgcacgagaaggtgctggccatcgtgaacgcgctggtggagaacaaggccatccgcggcgacgag
gccgggctggtgtacaacgcgctgctggagcgcgtggcccgctacaacagcaccaacgtgcagac
gaacctggaccgcatggtgaccgacgtgcgcgaggcggtgtcgcagcgcgagcggttccaccgcg
agtcgaacctgggctccatggtggcgctgaacgccttcctgagcacgcagcccgccaacgtgccc
cggggccaggaggactacaccaacttcatcagcgcgctgcggctgatggtggccgaggtgcccca
gagcgaggtgtaccagtcggggccggactacttcttccagaccagtcgccagggcttgcagaccg
tgaacctgagccaggctttcaagaacttgcagggactgtggggcgtgcaggccccggtcggggac
cgcgcgacggtgtcgagcctgctgacgccgaactcgcgcctgctgctgctgctggtggcgccctt
cacggacagcggcagcgtgagccgcgactcgtacctgggctacctgcttaacctgtaccgcgagg
ccatcgggcaggcgcacgtggacgagcagacctaccaggagatcacccacgtgagccgcgcgctg
ggccaggaggacccgggcaacctggaggccaccctgaacttcctgctgaccaaccggtcgcagaa
gatcccgccccagtacgcgctgagcaccgaggaggagcgcatcctgcgctacgtgcagcagagcg
tggggctgttcctgatgcaggagggggccacgcccagcgccgcgctcgacatgaccgcgcgcaac
atggagcccagcatgtacgctcgcaaccgcccgttcatcaataagctgatggactacttgcatcg
ggcggccgccatgaactcggactactttaccaacgccatcttgaacccgcactggctcccgccgc
ccgggttctacacgggcgagtacgacatgcccgaccccaacgacgggttcctgtgggacgacgtg
gacagcagcgtgttctcgccgcgccccgccaccaccgtgtggaagaaagagggcggggaccggcg
gccgtcctcggcgctgtccggtcgcgcgggtgctgccgcggcggtgcctgaggccgccagcccct
tcccgagcctgcccttttcgctgaacagcgtgcgcagcagcgagctgggtcggctgacgcggccg
cgcctgctgggcgaggaggagtacctgaacgactccttgttgaggcccgagcgcgagaagaactt
ccccaataacgggatagagagcctggtggacaagatgagccgctggaagacgtacgcgcacgagc
acagggacgagccccgagctagcagcagcgcaggcacccgtagacgccagcgacacgacaggcag
cggggtctggtgtgggacgatgaggattccgccgacgacagcagcgtgttggacttgggtgggag
tggtggtggtaacccgttcgctcacttgcgcccccgtatcgggcgcctgatgtaagaatctgaaa
aaataaaaaacggtactcaccaaggccatggcgaccagcgtgcgttcttctctgttgtttgtagt
agtatgatgaggcgcgtgtacccggagggtcctcctccctcgtacgagagcgtgatgcagcaggc
ggtggcggcggcgatgcagcccccgctggaggcgccttacgtgcccccgcggtacctggcgccta
cggaggggcggaacagcattcgttactcggagctggcacccttgtacgataccacccggttgtac
ctggtggacaacaagtcggcggacatcgcctcgctgaactaccagaacgaccacagcaacttcct
gaccaccgtggtgcagaacaacgatttcacccccacggaggccagcacccagaccatcaactttg
acgagcgctcgcggtggggcggccagctgaaaaccatcatgcacaccaacatgcccaacgtgaac
gagttcatgtacagcaacaagttcaaggcgcgggtgatggtctcgcgcaagacccccaatggggt
cgcggtggatgagaattatgatggtagtcaggacgagctgacttacgagtgggtggagtttgagc
tgcccgagggcaacttctcggtgaccatgaccatcgatctgatgaacaacgccatcatcgacaac
tacttggcggtggggcgtcagaacggggtgctggagagcgacatcggcgtgaagttcgacacgcg
caacttccggctgggctgggaccccgtgaccgagctggtgatgccgggcgtgtacaccaacgagg
ccttccaccccgacatcgtcctgctgcccggctgcggcgtggacttcaccgagagccgcctcagc
aacctgctgggcatccgcaagcggcagcccttccaggagggcttccagatcctgtacgaggacct
ggaggggggcaacatccccgcgctcttggatgtcgaagcctatgagaaaagcaaggaggaggccg
ccgcagcggcgaccgcagccgtggccaccgcctctaccgaggtgcggggcgataattttgctagc
gccgcggcagtggccgaggcggctgaaaccgaaagtaagatagtcatccagccggtggagaagga
cagcaaggacaggagctacaacgtgctcgcggacaagaaaaacaccgcctaccgcagctggtacc
tggcctacaactacggcgaccccgagaagggcgtgcgctcctggacgctgctcaccacctcggac
gtcacctgcggcgtggagcaagtctactggtcgctgcccgacatgatgcaagacccggtcacctt
ccgctccacgcgtcaagttagcaactacccggtggtgggcgccgagctcctgcccgtctactcca
agagcttcttcaacgagcaggccgtctactcgcagcagctgcgcgccttcacctcgctcacgcac
gtcttcaaccgcttccccgagaaccagatcctcgtccgcccgcccgcgcccaccattaccaccgt
cagtgaaaacgttcctgctctcacagatcacgggaccctgccgctgcgcagcagtatccggggag
tccagcgcgtgaccgtcactgacgccagacgccgcacctgcccctacgtctacaaggccctgggc
gtagtcccagcaagatgtacggaggcgctcgccaacgctccacgcaacaccccgtgcgcgtgcgc
gggcacttccgcgctccctggggcgccctcaagggccgcgtgcgctcgcgcaccaccgtcgacga
cgtgatcgaccaggtggtggccgacgcgcgcaactacacgcccgccgccgcgcccgcctccaccg
tggacgccgtcatcgacagcgtggtggccgatgcgcgccggtacgcccgcgccaagagccggcgg
cggcgcatcgcccggcggcaccggagcacccccgccatgcgcgcggcgcgagccttgctgcgcag
ggccaggcgcacgggacgcagggccatgctcagggcggccagacgcgcggcctccggcagcagca
gcgccggcaggacccgcagacgcgcggccacggcggcggcggcggccatcgccagcatgtcccgc
ccgcggcgcggcaacgtgtactgggtgcgcgacgccgccaccggtgtgcgcgtgcccgtgcgcac
ccgcccccctcgcacttgaagatgctgacttcgcgatgttgatgtgtcccagcggcgaggaggat
gtccaagcgcaaatacaaggaagagatgctccaggtcatcgcgcctgagatctacggccccgcgg
tgaaggaggaaagaaagccccgcaaactgaagcgggtcaaaaaggacaaaaaggaggaggaagat
gtggacggactggtggagtttgtgcgcgagttcgccccccggcggcgcgtgcagtggcgcgggcg
gaaagtgaaaccggtgctgcggcccggcaccacggtggtcttcacgcccggcgagcgttccggct
ccgcctccaagcgctcctacgacgaggtgtacggggacgaggacatcctcgagcaggcggtcgag
cgtctgggcgagtttgcttacggcaagcgcagccgccccgcgcccttgaaagaggaggcggtgtc
catcccgctggaccacggcaaccccacgccgagcctgaagccggtgaccctgcagcaggtgctgc
cgagcgcggcgccgcgccggggcttcaagcgcgagggcggcgaggatctgtacccgaccatgcag
ctgatggtgcccaagcgccagaagctggaggacgtgctggagcacatgaaggtggaccccgaggt
gcagcccgaggtcaaggtgcggcccatcaagcaggtggccccgggcctgggcgtgcagaccgtgg
acatcaagatccccacggagcccatggaaacgcagaccgagcccgtgaagcccagcaccagcacc
atggaggtgcagacggatccctggatgccggcgccggcttccaccactcgccgaagacgcaagta
cggcgcggccagcctgctgatgcccaactacgcgctgcatccttccatcatccccacgccgggct
accgcggcacgcgcttctaccgcggctacaccagcagccgccgcaagaccaccacccgccgccgc
cgtcgtcgcacccgccgcagcagcaccgcgacttccgccgccgccctggtgcggagagtgtaccg
cagcgggcgcgagcctctgaccctgccgcgcgcgcgctaccacccgagcatcgccatttaactct
gccgtcgcctcctacttgcagatatggccctcacatgccgcctccgcgtccccattacgggctac
cgaggaagaaagccgcgccgtagaaggctgacggggaacgggctgcgtcgccatcaccaccggcg
gcggcgcgccatcagcaagcggttggggggaggcttcctgcccgcgctgatccccatcatcgccg
cggcgatcggggcgatccccggcatagcttccgtggcggtgcaggcctctcagcgccactgagac
acagcttggaaaatttgtaataaaaaaatggactgacgctcctggtcctgtgatgtgtgttttta
gatggaagacatcaatttttcgtccctggcaccgcgacacggcacgcggccgtttatgggcacct
ggagcgacatcggcaacagccaactgaacgggggcgccttcaattggagcagtctctggagcggg
cttaagaatttcgggtccacgctcaaaacctatggcaacaaggcgtggaacagcagcacagggca
ggcgctgagggaaaagctgaaagagcagaacttccagcagaaggtggtcgatggcctggcctcgg
gcatcaacggggtggtggacctggccaaccaggccgtgcagaaacagatcaacagccgcctggac
gcggtcccgcccgcggggtccgtggagatgccccaggtggaggaggagctgcctcccctggacaa
gcgcggcgacaagcgaccgcgtcccgacgcggaggagacgctgctgacgcacacggacgagccgc
ccccgtacgaggaggcggtgaaactgggtctgcccaccacgcggcccgtggcgcctctggccacc
ggggtgctgaaacccagcagcagcagccagcccgcgaccctggacttgcctccgcctgcttcccg
cccctccacagtggctaagcccctgccgccggtggccgtcgcgtcgcgcgccccccgaggccgcc
cccaggcgaactggcagagcactctgaacagcatcgtgggtctgggagtgcagagtgtgaagcgc
cgccgctgctattaaaagacactgtagcgcttaacttgcttgtctgtgtgtatatgtatgtccgc
cgaccagaaggaggaagaggcgcgtcgccgagttgcaagatggccaccccatcgatgctgcccca
gtgggcgtacatgcacatcgccggacaggacgcttcggagtacctgagtccgggtctggtgcagt
tcgcccgcgccacagacacctacttcagtctggggaacaagtttaggaaccccacggtggcgccc
acgcacgatgtgaccaccgaccgcagccagcggctgacgctgcgcttcgtgcccgtggaccgcga
ggacaacacctactcgtacaaagtgcgctacacgctggccgtgggcgacaaccgcgtgctggaca
tggccagcacctactttgacatccgcggcgtgctggatcgggggcccagcttcaaaccctactcc
ggcaccgcctacaacagcctggctcccaagggagcgcccaacacttgccagtggacatataaagc
tggtgatactgatacagaaaaaacctatacatatggaaatgcacctgtgcaaggcattagcatta
caaaggatggtattcaacttggaactgacagcgatggtcaggcaatctatgcagacgaaacttat
caaccagagcctcaagtgggtgatgctgaatggcatgacatcactggtactgatgaaaaatatgg
aggcagagctcttaagcctgacaccaaaatgaagccttgctatggttcttttgccaagcctacca
ataaagaaggaggccaggcaaatgtgaaaaccgaaacaggcggtaccaaagaatatgacattgac
atggcattcttcgataatcgaagtgcagctgccgccggcctagccccagaaattgttttgtatac
tgagaatgtggatctggaaactccagatacccatattgtatacaaggcaggtacagatgacagta
gctcttctatcaatttgggtcagcagtccatgcccaacagacccaactacattggcttcagagac
aactttatcggtctgatgtactacaacagcactggcaatatgggtgtactggctggacaggcctc
ccagctgaatgctgtggtggacttgcaggacagaaacaccgaactgtcctaccagctcttgcttg
actctctgggtgacagaaccaggtatttcagtatgtggaatcaggcggtggacagttatgacccc
gatgtgcgcattattgaaaatcacggtgtggaggatgaacttcctaactattgcttccccctgga
tgctgtgggtagaactgatacttaccagggaattaaggccaatggtgataatcaaaccacctgga
ccaaagatgatactgttaatgatgctaatgaattgggcaagggcaatcctttcgccatggagatc
aacatccaggccaacctgtggcggaacttcctctacgcgaacgtggcgctgtacctgcccgactc
ctacaagtacacgccggccaacatcacgctgcccaccaacaccaacacctacgattacatgaacg
gccgcgtggtggcgccctcgctggtggacgcctacatcaacatcggggcgcgctggtcgctggac
cccatggacaacgtcaaccccttcaaccaccaccgcaacgcgggcctgcgataccgctccatgct
cctgggcaacgggcgctacgtgcccttccacatccaggtgccccaaaagtttttcgccatcaaga
gcctcctgctcctgcccgggtcctacacctacgagtggaacttccgcaaggacgtcaacatgatc
ctgcagagctccctcggcaacgacctgcgcacggacggggcctccatcgccttcaccagcatcaa
cctctacgccaccttcttccccatggcgcacaacaccgcctccacgctcgaggccatgctgcgca
acgacaccaacgaccagtccttcaacgactacctctcggcggccaacatgctctaccccatcccg
gccaacgccaccaacgtgcccatctccatcccctcgcgcaactgggccgccttccgcggctggtc
cttcacgcgcctcaagacccgcgagacgccctcgctcggctccgggttcgacccctacttcgtct
actcgggctccatcccctacctcgacggcaccttctacctcaaccacaccttcaagaaggtctcc
atcaccttcgactcctccgtcagctggcccggcaacgaccgcctcctgacgcccaacgagttcga
aatcaagcgcaccgtcgacggagaggggtacaacgtggcccagtgcaacatgaccaaggactggt
tcctggtccagatgctggcccactacaacatcggctaccagggcttctacgtgcccgagggctac
aaggaccgcatgtactccttcttccgcaacttccagcccatgagccgccaggtcgtggacgaggt
caactacaaggactaccaggccgtcaccctggcctaccagcacaacaactcgggcttcgtcggct
acctcgcgcccaccatgcgccagggccagccctaccccgccaactacccctacccgctcatcggc
aagagcgccgtcgccagcgtcacccagaaaaagttcctctgcgaccgggtcatgtggcgcatccc
cttctccagcaacttcatgtccatgggcgcgctcaccgacctcggccagaacatgctctacgcca
actccgcccacgcgctagacatgaatttcgaagtcgaccccatggatgagtccacccttctctat
gttgtcttcgaagtcttcgacgtcgtccgagtgcaccagccccaccgcggcgtcatcgaggccgt
ctacctgcgcacgcccttctcggccggcaacgccaccacctaagcctcttgcttcttgcaagatg
acggcctgcgcgggctccggcgagcaggagctcagggccatcctccgcgacctgggctgcgggcc
ctgcttcctgggcaccttcgacaagcgcttcccgggattcatggccccgcacaagctggcctgcg
ccatcgtcaacacggccggccgcgagaccgggggcgagcactggctggccttcgcctggaacccg
cgctcccacacctgctacctcttcgaccccttcgggttctcggacgagcgcctcaagcagatcta
ccagttcgagtacgagggcctgctgcgtcgcagcgccctggccaccgaggaccgctgcgtcaccc
tggaaaagtccacccagaccgtgcagggtccgcgctcggccgcctgcgggctcttctgctgcatg
ttcctgcacgccttcgtgcactggcccgaccgccccatggacaagaaccccaccatgaacttgct
gacgggggtgcccaacggcatgctccagtcgccccaggtggaacccaccctgcgccgcaaccagg
aggcgctctaccgcttcctcaacgcccactccgcctactttcgctcccaccgcgcgcgcatcgag
aaggccaccgccttcgaccgcatgaatcaagacatgtaatccggtgtgtgtatgtgaatgcttta
ttcatcataataaacagcacatgtttatgccaccttctctgaggctctgactttatttagaaatc
gaaggggttctgccggctctcggcatggcccgcgggcagggatacgttgcggaactggtacttgg
gcagccacttgaactcggggatcagcagcttcggcacggggaggtcggggaacgagtcgctccac
agcttgcgcgtgagttgcagggcgcccagcaggtcgggcgcggagatcttgaaatcgcagttggg
acccgcgttctgcgcgcgagagttacggtacacggggttgcagcactggaacaccatcagggccg
ggtgcttcacgctcgccagcaccgtcgcgtcggtgatgccctccacgtccagatcctcggcgttg
gccatcccgaagggggtcatcttgcaggtctgccgccccatgctgggcacgcagccgggcttgtg
gttgcaatcgcagtgcagggggatcagcatcatctgggcctgctcggagctcatgcccgggtaca
tggccttcatgaaagcctccagctggcggaaggcctgctgcgccttgccgccctcggtgaagaag
accccgcaggacttgctagagaactggttggtggcgcagccagcgtcgtgcacgcagcagcgcgc
gtcgttgttggccagctgcaccacgctgcgcccccagcggttctgggtgatcttggcccggtcgg
ggttctccttcagcgcgcgctgcccgttctcgctcgccacatccatctcgatcgtgtgctccttc
tggatcatcacggtcccgtgcaggcaccgcagcttgccctcggcctcggtgcacccgtgcagcca
cagcgcgcagccggtgctctcccagttcttgtgggcgatctgggagtgcgagtgcacgaagccct
gcaggaagcggcccatcatcgtggtcagggtcttgttgctggtgaaggtcagcggaatgccgcgg
tgctcctcgttcacatacaggtggcagatacggcggtacacctcgccctgctcgggcatcagctg
gaaggcggacttcaggtcgctctccacgcggtaccggtccatcagcagcgtcatcacttccatgc
ccttctcccaggccgaaacgatcggcaggctcagggggttcttcaccgttgtcatcttagtcgcc
gccgccgaagtcagggggtcgttctcgtccagggtctcaaacactcgcttgccgtccttctcggt
gatgcgcacggggggaaagctgaagcccacggccgccagctcctcctcggcctgcctttcgtcct
cgctgtcctggctgatgtcttgcaaaggcacatgcttggtcttgcggggtttctttttgggcggc
agaggcggcggcggagacgtgctgggcgagcgcgagttctcgctcaccacgactatttcttctcc
ttggccgtcgtccgagaccacgcggcggtaggcatgcctcttctggggcagaggcggaggcgacg
ggctctcgcggttcggcgggcggctggcagagccccttccgcgttcgggggtgcgctcctggcgg
cgctgctctgactgacttcctccgcggccggccattgtgttctcctagggagcaagcatggagac
tcagccatcgtcgccaacatcgccatctgcccccgccgccgccgacgagaaccagcagcagcaga
atgaaagcttaaccgccccgccgcccagccccacctccgacgccgcagccccagacatgcaagag
atggaggaatccatcgagattgacctgggctacgtgacgcccgcggagcacgaggaggagctggc
agcgcgcttttcagccccggaagagaaccaccaagagcagccagagcaggaagcagagagcgagc
agaaccaggctgggctcgagcatggcgactacctgagcggggcagaggacgtgctcatcaagcat
ctggcccgccaatgcatcatcgtcaaggacgcgctgctcgaccgcgccgaggtgcccctcagcgt
ggcggagctcagccgcgcctacgagcgcaacctcttctcgccgcgcgtgccccccaagcgccagc
ccaacggcacctgcgagcccaacccgcgcctcaacttctacccggtcttcgcggtgcccgaggcc
ctggccacctaccacctctttttcaagaaccaaaggatccccgtctcctgccgcgccaaccgcac
ccgcgccgacgccctgctcaacctgggccccggcgcccgcctacctgatatcgcctccttggaag
aggttcccaagatcttcgagggtctgggcagcgacgagactcgggccgcgaacgctctgcaagga
agcggagaggagcatgagcaccacagcgccctggtggagttggaaggcgacaacgcgcgcctggc
ggtcctcaagcgcacggtcgagctgacccacttcgcctacccggcgctcaacctgccccccaagg
tcatgagcgccgtcatggaccaggtgctcatcaagcgcgcctcgcccctctcggaggaggagatg
caggaccccgagagctcggacgagggcaagcccgtggtcagcgacgagcagctggcgcgctggct
gggagcgagtagcaccccccagagcctggaagagcggcgcaagctcatgatggccgtggtcctgg
tgaccgtggagctggagtgtctgcgccgcttcttcgccgacgcggagaccctgcgcaaggtcgag
gagaacctgcactacctcttcagacacgggttcgtgcgccaggcctgcaagatctccaacgtgga
gctgaccaacctggtctcctacatgggcatcctgcacgagaaccgcctggggcagaacgtgctgc
acaccaccctgcgcggggaggcccgccgcgactacatccgcgactgcgtctacctgtacctctgc
cacacctggcagacgggcatgggcgtgtggcagcagtgcctggaggagcagaacctgaaagagct
ctgcaagctcctgcagaagaacctcaaggccctgtggaccgggttcgacgagcgcaccaccgccg
cggacctggccgacctcatcttccccgagcgcctgcggctgacgctgcgcaacgggctgcccgac
tttatgagccaaagcatgttgcaaaactttcgctctttcatcctcgaacgctccgggatcctgcc
cgccacctgctccgcgctgccctcggacttcgtgccgctgaccttccgcgagtgccccccgccgc
tctggagccactgctacctgctgcgcctggccaactacctggcctaccactcggacgtgatcgag
gacgtcagcggcgagggcctgctcgagtgccactgccgctgcaacctctgcacgccgcaccgctc
cctggcctgcaacccccagctgctgagcgagacccagatcatcggcaccttcgagttgcaaggcc
ccggcgagggcaaggggggtctgaaactcaccccggggctgtggacctcggcctacttgcgcaag
ttcgtgcccgaggactaccatcccttcgagatcaggttctacgaggaccaatcccagccgcccaa
ggccgagctgtcggcctgcgtcatcacccagggggccatcctggcccaattgcaagccatccaga
aatcccgccaagaatttctgctgaaaaagggccacggggtctacttggacccccagaccggagag
gagctcaaccccagcttcccccaggatgccccgaggaagcagcaagaagctgaaagtggagctgc
cgccgccgccggaggatttggaggaagactgggagagcagtcaggcagaggaggaggagatggaa
gactgggacagcactcaggcagaggaggacagcctgcaagacagtctggaggaggaagacgaggt
ggaggaggcagaggaagaagcagccgccgccagaccgtcgtcctcggcggaggaggagaaagcaa
gcagcacggataccatctccgctccgggtcggggtcgcggcggccgggcccacagtagatgggac
gagaccgggcgcttcccgaaccccaccacccagaccggtaagaaggagcggcagggatacaagtc
ctggcgggggcacaaaaacgccatcgtctcctgcttgcaagcctgcgggggcaacatctccttca
cccggcgctacctgctcttccaccgcggggtgaacttcccccgcaacatcttgcattactaccgt
cacctccacagcccctactactgtttccaagaagaggcagaaacccagcagcagcagcagcagca
gaaaaccagcggcagcagctagaaaatccacagcggcggcaggtggactgaggatcgcggcgaac
gagccggcgcagacccgggagctgaggaaccggatctttcccaccctctatgccatcttccagca
gagtcgggggcaagagcaggaactgaaagtcaagaaccgttctctgcgctcgctcacccgcagtt
gtctgtatcacaagagcgaagaccaacttcagcgcactctcgaggacgccgaggctctcttcaac
aagtactgcgcgctcactcttaaagagtagcccgcgcccgcccacacacggaaaaaggcgggaat
tacgtcaccacctgcgcccttcgcccgaccatcatcatgagcaaagagattcccacgccttacat
gtggagctaccagccccagatgggcctggccgccggcgccgcccaggactactccacccgcatga
actggctcagtgccgggcccgcgatgatctcacgggtgaatgacatccgcgcccaccgaaaccag
atactcctagaacagtcagcgatcaccgccacgccccgccatcaccttaatccgcgtaattggcc
cgccgccctggtgtaccaggaaattccccagcccacgaccgtactacttccgcgagacgcccagg
ccgaagtccagctgactaactcaggtgtccagctggccggcggcgccgccctgtgtcgtcaccgc
cccgctcagggtataaagcggctggtgatccgaggcagaggcacacagctcaacgacgaggtggt
gagctcttcgctgggtctgcgacctgacggagtcttccaactcgccggatcggggagatcttcct
tcacgcctcgtcaggccgtcctgactttggagagttcgtcctcgcagccccgctcgggtggcatc
ggcactctccagttcgtggaggagttcactccctcggtctacttcaaccccttctccggctcccc
cggccactacccggacgagttcatcccgaacttcgacgccatcagcgagtcggtggacggctacg
attgaatgtcccatggtggcgcggctgacctagctcggcttcgacacctggaccactgccgccgc
ttccgctgcttcgctcgggatctcgccgagtttgcctactttgagctgcccgaggagcaccctca
gggcccggcccacggagtgcggatcgtcgtcgaagggggtctcgactcccacctgcttcggatct
tcagccagcgtccgatcctggccgagcgcgagcaaggacagacccttctgaccctgtactgcatc
tgcaaccaccccggcctgcatgaaagtctttgttgtctgctgtgtactgagtataataaaagctg
agatcagcgactactccggacttccgtgtgttcctgctatcaaccagtccctgttcttcaccggg
aacgagaccgagctccagctccagtgtaagccccacaagaagtacctcacctggctgttccaggg
ctctccgatcgccgttgtcaaccactgcgacaacgacggagtcctgctgagcggccctgccaacc
ttactttttccacccgcagaagcaagctccagctcttccaacccttcctccccgggacctatcag
tgcgtctcgggaccctgccatcacaccttccacctgatcccgaataccacagcgtcgctccccgc
tactaacaaccaaactacccaccaacgccaccgtcgcgaccgcggacatgtacagagctcgagaa
gtactaggccacaatacatgcccatattagactatgaggccgagccacagcgacccatgctcccc
gctattagttacttcaatctaaccggcggagatgactgacccactggccaacaacaacgtcaacg
accttctcctggacatggacggccgcgcctcggagcagcgactcgcccaacttcgcattcgccag
cagcaggagagagccgtcaaggagctgcaggacggcatagccatccaccagtgcaagaaaggcat
cttctgcctggtgaaacaggccaagatctcctacgaggtcaccccgaccgaccatcgcctctcct
acgagctcctgcagcagcgccagaagttcacctgcctggtcggagtcaaccccatcgtcatcacc
cagcagtcgggcgataccaaggggtgcatccactgctcctgcgactcccccgactgcgtccacac
tctgatcaagaccctctgcggcctccgcgacctcctccccatgaactaatcacccccttatccag
tgaaataaatatcatattgatgatgatttaaataaaaaataatcatttgatttgaaataaagata
caatcatattgatgatttgagttttaaaaaataaagaatcacttacttgaaatctgataccaggt
ctctgtccatgttttctgccaacaccacctcactcccctcttcccagctctggtactgcagaccc
cggcgggctgcaaacttcctccacacgctgaaggggatgtcaaattcctcctgtccctcaatctt
cattttatcttctatcagacccccccttcgtctcttcagatggattccaagagaagcccctgggg
gtgctgtccctgcgactggctgaccccgtcaccaccaagaacggggaaatcaccctcaagctggg
agagggggtggacctcgactcctcgggaaaactcatctccaacacggccaccaaggccgccgccc
ctctcagtttttccaacaacaccatttcccttaacatggatacccctctttataccaaagatgga
aaattatccttacaagtttctccaccgttaaacatattaaaatcaaccattctgaacacattagc
tgtagcttatggatcaggtttaggactgagtggtggcactgctcttgcagtacagttggcctctc
cactcacttttgatgaaaaaggaaatattaaaattaacctagccagtggtccattaacagttgat
gcaagtcgacttagtatcaactgcaaaagaggggtcactgtcactacctcaggagatgcaattga
aagcaacataagctggcctaaaggtataagatttgaaggtaatggcatagctgcaaacattggca
gaggattggaatttggaaccactagtacagagactgatgtcacagatgcatacccaattcaagtt
aaattgggtactggccttacctttgacagtacaggcgccattgttgcttggaacaaagaggatga
taaacttacattatggaccacagccgacccctcgccaaattgcaaaatatactctgaaaaagatg
ccaaactcacactttgcttgacaaagtgtggaagtcaaattctgggtactgtgactgtattggca
gtgaataatggaagtctcaacccaatcacaaacacagtaagcactgcactcgtctccctcaagtt
tgatgcaagtggagttttgctaagcagctccacattagacaaagaatattggaacttcagaaagg
gagatgttacacctgctgagccctatactaatgctataggttttatgcctaacataaaggcctat
cctaaaaacacatctgcagcttcaaaaagccatattgtcagtcaagtttatctcaatggggatga
ggccaaaccactgatgctgattattacttttaatgaaactgaggatgcaacttgcacctacagta
tcacttttcaatggaaatgggatagtactaagtacacaggtgaaacacttgctaccagctccttc
accttctcctacatcgcccaagaatgaacactgtatcccaccctgcataggattcgagcagttat
ttttcctccaccctcccaggacatggaatacaccaccctctccccccgcacagccttgaacatct
gaatgccattggtgatggacatgcttttggtctccacgttccacacagtttcagagcgagccagt
ctcgggtcggtcagggagatgaaaccctccgggcactcccgcatctgcacctcacagctcaacag
ctgaggattgtcctcggtggtcgggatcacggttatctggaagaagcagaagagcggcggtggga
atcatagtccgcgaacgggatcggccggtggtgtcgcatcaggccccgcagcagtcgctgccgcc
gccgctccgtcaagctgctgctcagggggtccgggtccagggactccctcagcatgatgcccacg
gccctcagcatcagtcgtctggtgcggcgggcgcagcagcgcatgcggatctcgctcaggtcgct
gcagtacgtgcaacacaggaccaccaggttgttcaacagtccatagttcaacacgctccagccga
aactcatcgcgggaaggatgctacccacgtggccgtcgtaccagatcctcaggtaaatcaagtgg
cgctccctccagaacacgctgcccacgtacatgatctccttgggcatgtggcggttcaccacctc
ccggtaccacatcaccctctggttgaacatgcagccccggatgatcctgcggaaccacagggcca
gcaccgccccgcccgccatgcagcgaagagaccccgggtcccggcaatggcaatggaggacccac
cgctcgtacccgtggatcatctgggagctgaacaagtctatgttggcacagcacaggcatatgct
catgcatctcttcagcactctcagctcctcgggggtcaaaaccatatcccagggcacggggaact
cttgcaggacagcgaaccccgcagaacagggcaatcctcgcacataacttacattgtgcatggac
agggtatcgcaatcaggcagcaccgggtgatcctccaccagagaagcgcgggtctcggtctcctc
acagcgtggtaagggggccggccgatacgggtgatggcgggacgcggctgatcgtgttcgcgacc
gtgtcatgatgcagttgctttcggacattttcgtacttgctgtagcagaacctggtccgggcgct
gcacaccgatcgccggcggcggtcccggcgcttggaacgctcggtgttgaaattgtaaaacagcc
actctctcagaccgtgcagcagatctagggcctcaggagtgatgaagatcccatcatgcctgata
gctctgatcacatcgaccaccgtggaatgggccagacccagccagatgatgcaattttgttgggt
ttcggtgacggcgggggagggaagaacaggaagaaccatgattaacttttaatccaaacggtctc
ggagcacttcaaaatgaaggtcgcggagatggcacctctcgcccccgctgtgttggtggaaaata
acagccaggtcaaaggtgatacggttctcgagatgttccacggtggcttccagcaaagcctccac
gcgcacatccagaaacaagacaatagcgaaagcgggagggttctctaattcctcaatcatcatgt
tacactcctgcaccatccccagataattttcatttttccagccttgaatgattcgaactagttcc
tgaggtaaatccaagccagccatgataaagagctcgcgcagagcgccctccaccggcattcttaa
gcacaccctcataattccaagatattctgctcctggttcacctgcagcagattgacaagcggaat
atcaaaatctctgccgcgatccctaagctcctccctcagcaataactgtaagtactctttcatat
cctctccgaaatttttagccataggaccaccaggaataagattagggcaagccacagtacagata
aaccgaagtcctccccagtgagcattgccaaatgcaagactgctataagcatgctggctagaccc
ggtgatatcttccagataactggacagaaaatcacccaggcaatttttaagaaaatcaacaaaag
aaaaatcctccaggtgcacgtttagagcctcgggaacaacgatgaagtaaatgcaagcggtgcgt
tccagcatggttagttagctgatctgtaaaaaacaaaaaataaaacattaaaccatgctagcctg
gcgaacaggtgggtaaatcgttctctccagcaccaggcaggccacggggtctccggcgcgaccct
cgtaaaaattgtcgctatgattgaaaaccatcacagagagacgttcccggtggccggcgtgaatg
attcgacaagatgaatacaccgatgccatgcggatgaagcacaaaatcctcaggtgcgtacaaaa
tgtaattactcccctcctgcacaggcagcgaagcccccgatccctccagatacacatacaaagcc
tcagcgtccatagcttaccgagcagcagcacacaacaggcgcaagagtcagagaaaggctgagct
ctaacctgtccacccgctctctgctcaatatatagcccagatctacactgacgtaaaggccaaag
tctaaaaatacccgccaaataatcacacacgcccagcacacgcccagaaaccggtgacacactca
aaaaaatacgcgcacttcctcaaacgcccaaactgccgtcatttccgggttcccacgctacgtca
tcggaattcgactttcaaattccgtcgaccgttaaaaacgtcacccgccccgcccctaacggtcg
cccgtctctcggccaatcaccttcctccctccccaaattcaaacagctcatttgcatattaacgc
gcaccaaaagtttgaggtatattattgatgatg

### Example 6: Genetic Stability and Protein Expression

Genetic Stability: All of the vectors were genetically stable as determined by analyzing purified viral DNA by restriction enzyme digest followed by gel electrophoresis upon 12 sequential passages on HEK 293 cells.

Protein Expression: HEK 293 cells were infected for 48 hours with the different vectors. A cell lysate was prepared. Proteins were separated by SDS-PAGE. Bands spanning protein of approximate sizes from 110-160 kD was cut from the gel, protein were eluted and analyzed by Mass Spectrophotometry. The results (shown below) showed that for each of the cell lysates, peptides derived from gp140 could be detected. Sequences detected from the cell lysates within gp140 of the 3 different HIV clades expressed by the two adenoviral vector serotypes are underlined and in bold. The results indicated the AdC7 vector most likely only expresses low levels of the clade B gp140 protein.

Gp140 Clade BC: Accession number, KC492738(SEQ ID NO: 4)
AdC6 gp140 BC
(SEQ ID NO: 4)
MRVMGIRRNCQHLWRWGIMLLGMLMICSVVGNLWVTVYYGVPVWK [symbol] [symbol]
AYDTEVHNVWATHACVPTDPNPQEMVLENVTENFNMWK [symbol] [symbol]
LTPLCVTLKCKNVSSNSTETPKLRGNSSETYKDEEMK
NCSFNATTILRDK [symbol] LDIAPLLLNSSENSSAYYSLINCNTSAIT
QACPKVSFDPIPIHYCTPAGYAILKCNDKKFNGTGPCSNVSTVQCTHGIKPV
VSTQLLLNGSLAEGEVIIRSKNLTDNAKTIIVQLNRSVEIVCTRPNNNTRKS
IRI [symbol] QAHCNISEDMWNETLHWVSRKLAEHFPNRTIN
FTSSSGGDLEIATHSFNCRGEFFYCNTSRLFNGTYMFNGTRGNSSSNSTITI
PCRIK [symbol] AMYAPPIEGNLTCRSNITGLLLVRDGGDNTNKTEIF
RPQGGDMRDNWRSELYKYK [symbol] LTVQAR [symbol]
AIEAQQHLLQLTVWGIKQLQTRVLAIERYLK [symbol] [symbol]
LICTTAVPWNSSWSNKTQDEIWNNLTWMQWDKEISNYTD
TIYK [symbol] [symbol] NEK [symbol] NLWSWFDITNWLW
AdC7 gp140 BC
(SEQ ID NO: 4)
MRVMGIRRNCQHLWRWGIMLLGMLMICSVVGNLWVTVYYGVPVWK [symbol] [symbol]
AYDTEVHNVWATHACVPTDPNPQEMVLENVTENFNMWK [symbol] [symbol]
LTPLCVTLKCKNVSSNSTETPKLRGNSSETYKDEEMKN
CSFNATTILRDKKQEVYALFYKLDIAPLLLNSSENSSAYYSLINCNTSAITQACPK
VSFDPIPIHYCTPAGYAILKCNDKKFNGTGPCSNVSTVQCTHGIKPVVSTQLLLNG
SLAEGEVIIRSKNLTDNAKTIIVQLNRSVEIVCTRPNNNTRKSIR [symbol]
QAHCNISEDMWNETLHWVSRKLAEHFPNRTINFTSS
SGGDLEIATHSFNCRGEFFYCNTSRLFNGTYMFNGTRGNSSSNSTITIPCRIKQII
MWNQQVGRAMYAPPIEGNLTCRSNITGLLLVRDGGDNTNKTEIFRPQGGDMRDNWR
SELYKYK [symbol] LTVQAR [symbol]
ATEAQQHLLQLTVWGIKQLQTRVLAIERYLKDQQLLGIWGCSGKLICTTAV
PWNSSWSNKTQDEIWNNLTWMQWDKEISNYTDTIYKLLEDSQNQQERNEKDLLAL
DSWKNLWSWFDITNWLW
Gp140 Clade B: Accession number, HM215399 (SEQ ID NO: 2)
AdC6 GP140 B
(SEQ ID NO: 2)
MRVKGIRKNYQHLWRWGTMLLGMLMICSAAENLWVTVYYGVPVWKEATTTLFCA
SDAKAYDTEVHNIWATHACVPTDPNPQEVVLGNVTENFNMWKNDMVEQMHEDII
SLWDQSLKPCVKLTPLCVTLNCTNLRNTNNTSSNTSNMTEGGEIKNCSFDITTS
IRTKVKDYALFYELDIVAIDNTSYRLRQCNTSVITQACPK [symbol]
LKCNNKTFNGTGPCTNVSTVQCTHRIRPVVSTQLLL
NGSLAEEEVVIRSSNFTDNAKVIIVQLKESVEINCTRPNNNTRKSIPLGPGKAW
YTTGQIIGDIRQAHCNLSRAK [symbol] KKLREQFGNKTIIFNQSSGGDPEV
VTHSFNCGGEFFYCNTSQLFNSTWYNNSTWNDTNDTTENSTITLPCRIKQIVNM
WQEVGKAMYAPPIRGQIRCSSNITGLLLTRDGGKNESNTTETFRPGGGDMRDNW
RSELYKYKVVK [symbol] AKLTVQAR [symbol] [symbol] NLLR
AIEAQQHLLQLTVWGIKQLQARVLAVERYLKDQQLLGIWGCSGKLICTTAVPWN
VSWSNRSLSEIWDNMTWMEWEREIGNYTK [symbol] [symbol]
WASLWNWFNITNWLW
AdC7 GP140 B
(SEQ ID NO: 2)
MRVKGIRKNYQHLWRWGTMLLGMLMICSAAENLWVTVYYGVPVWKEATTTLFCA
SDAKAYDTEVHNIWATHACVPTDPNPQEVVLGNVTENFNMWKNDMVEQMHEDII
SLWDQSLKPCVKLTPLCVTLNCTNLRNTNNTSSNTSNMTEGGEIKNCSFDITTS
IRTKVKDYALFYELDIVAIDNTSYRLRQCNTSVITQACPKISFEPIPIHYCTPA
GFAILKCNNKTFNGTGPCTNVSTVQCTHRIRPVVSTQLLLNGSLAEEEVVIRSS
NFTDNAKVIIVQLKESVEINCTRPNNNTRKSIPLGPGKAWYTTGQIIGDIRQAH
CNLSRAKWENTLQQITKKLREQFGNKTIIFNQSSGGDPEVVTHSFNCGGEFFYC
NTSQLFNSTWYNNSTWNDTNDTTENSTITLPCRIKQIVNMWQEVGKAMYAPPIR
GQIRCSSNITGLLLTRDGGKNESNTTETFRPGGGDMRDNWRSELYKYKVVKIEP
LGVAPTRAKLTVQARQLLSGIVQQQRNLLRAIEAQQHLLQLTVWGIKQLQARVL
AVERYLKDQQLLGIWGCSGKLICTTAVPWNVSWSNRSLSEIWDNMTWMEWEREI
GNYTK [symbol] LELLEWDKWASLWNWFNITNWLW
Gp140 Clade C: Accession number, KF835515 (SEQ ID NO: 3)
AdC6 GP140 C
(SEQ ID NO: 3)
MRVRGTQRNYPQWWIWGILGFWMLMICNVGGNLWVTVYYGVPVWK [symbol] [symbol]
AYENEVHNVWATHACVPTDPNPQEMVLENVTENFNMWK [symbol] [symbol]
TPLCVTLKCSNVTLKNNTVNSNETQYRKNCTFNTTT
ELKNRKQKVSAIFYRIDIVPLGNESSGNYRLINCNTSAITQACPKVSFDPIP
IHYCTPAGYALLKCNNKTFNGTGPCNNVSTVQCTHGIKPVVSTQLLLNGSLA
EEEIIIRSENLTNNVKTIIVHLNESVEIVCIRPGNNTRQSIRI [symbol]
QAHCNINGTKWNETLQGVGKKLAEHFPNKTIK [symbol]
GEFFYCDTSGLFNSTYNSTYVPNGTESKPNITI
QCRIK [symbol] AMYAPPIKGSITCKSNITGLLLVRDGGANTTEEIFR
PGGGDMRDNVVRSELYKY [symbol] LTVQAR [symbol] [symbol]
AIEAQQHMLQLTVWGIKQLQTRVLAIERYLKDQQLLGIWGCSGKLICTT
AVPWNSSWSNKTQDEIWKNMTWMQWDREINNYTNTIYSLLEESQNQQEKNEK
D [symbol] [symbol] NLWNWFDISNVVLW*
AdC7 GP140 C
(SEQ ID NO: 3)
MRVRGTQRNYPQWWIWGILGFWMLMICNVGGNLWVTVYYGVPVWKEATTTLFCA
SDAKAYENEVHNVWATHACVPTDPNPQEMVLENVTENFNMWKNEMVNQMHEDVI
SLWDQSLKPCVKLTPLCVTLKCSNVTLKNNTVNSNETQYRKNCTFNTTTELKNR
KQK [symbol] IDIVPLGNESSGNYRLINCNTSAITQACPKVSFDPIPIHYCTPA
GYALLKCNNKTFNGTGPCNNVSTVQCTHGIKPVVSTQLLLNGSLAEEEIIIRSE
NLTNNVKTIIVHLNESVEIVCIRPGNNTRQSIRI [symbol] QAH
CNINGTKWNETLQGVGKKLAEHFPNKTIK [symbol] GEFFYC
DTSGLFNSTYNSTYVPNGTESKPNITIQCRIKQIINMWQEVGRAMYAPPIKGSI
TCKSNITGLLLVRDGGANTTEEIFRPGGGDMRDNWRSELYKYKVVEIKPLGIAP
TEAKLTVQAR [symbol] AIEAQQHMLQLTVWGIKQLQTRVLAIER
YLKDQQLLGIWGCSGKLICTTAVPWNSSWSNKTQDEIWKNMTWMQWDREINNYT
NTIYSLLEESQNQQEKNEKDLLALDSWKNLWNWFDISNWLW*
Gp140 Clade BC: Accession number, KC492738 (SEQ ID NO: 4)
AdC6 GP140 BC
(SEQ ID NO: 4)
MRVMGIRRNCQHLWRWGIMLLGMLMICSVVGNLWVTVYYGVPVWK [symbol] [symbol]
AYDTEVHNVWATHACVPTDPNPQEMVLENVTENFNMWK [symbol] [symbol]
LTPLCVTLKCKNVSSNSTETPKLRGNSSETYKDEEMK
NCSFNATTILRDKK [symbol] LDIAPLLLNSSENSSAYYSLINCNTSAIT
QACPKVSFDPIPIHYCTPAGYAILKCNDKKFNGTGPCSNVSTVQCTHGIKPV
VSTQLLLNGSLAEGEVIIRSKNLTDNAKTIIVQLNRSVEIVCTRPNNNTRKS
IR [symbol] QAHCNISEDMWNETLHWVSRKLAEHFPNRTIN
FTSSSGGDLEIATHSFNCRGEFFYCNTSRLFNGTYMFNGTRGNSSSNSTITI
PCRIK [symbol] AMYAPPIEGNLTCRSNITGLLLVRDGGDNTNKTEIF
RPQGGDMRDNWRSELYKYK [symbol] LTVQAR [symbol]
AIEAQQHLLQLTVWGIKQLQTRVLAIERYLK [symbol] [symbol]
LICTTAVPWNSSWSNKTQDEIWNNLTWMQWDKEISNYTD
TIYK [symbol] [symbol] NEK [symbol] NLWSWFDITNWLW*
AdC7 GP140 BC
(SEQ ID NO: 4)
MRVMGIRRNCQHLWRWGIMLLGMLMICSVVGNLWVTVYYGVPVWK [symbol] [symbol]
AYDTEVHNVWATHACVPTDPNPQEMVLENVTENFNMWK [symbol] [symbol]
LTPLCVTLKCKNVSSNSTETPKLRGNSSETYKDEEMK
NCSFNATTILRDKK [symbol] LDIAPLLLNSSENSSAYYSLINCNTSAIT
QACPKVSFDPIPIHYCTPAGYAILKCNDKKFNGTGPCSNVSTVQCTHGIKPV
VSTQLLLNGSLAEGEVIIRSKNLTDNAKTIIVQLNRSVEIVCTRPNNNTRKS
IR [symbol] QAHCNISEDMWNETLHWVSRKLAEHFPNRTIN
FTSSSGGDLEIATHSFNCRGEFFYCNTSRLFNGTYMFNGTRGNSSSNSTITI
PCRIKQIINMWQQVGRAMYAPPIEGNLTCRSNITGLLLVRDGGDNTNKTEIF
RPQGGDMRDNWRSELYKYK [symbol] LTVQAR [symbol]
AIEAQQHLLQLTVWGIKQLQTRVLAIERYLKDQQLLG
IWGCSGKLICTTAVPWNSSWSNKTQDEIWNNLTWMQWDKEISNYTDTIYKLL
EDSQNQQERNEKDLLALDSWKNLWSWFDITNWLW

### REFERENCES

Carnathan D G, Wetzel K S, Yu J, Lee S T, Johnson B A, Paiardini M, Yan J, Morrow M P, Sardesai N Y, Weiner D B, Ertl H C, Silvestri G. Activated CD4+CCR5+ T cells in the rectum predict increased SIV acquisition in SIVGag/Tat-vaccinated rhesus macaques. Proc Natl Acad Sci USA. 2015 Jan. 13; 112(2):518-23.
Tuyishime S, Haut L H, Kurupati R K, Billingsley J M, Carnathan D, Gangahara S, Styles T M, Xiang Z, Li Y, Zopfs M, Liu Q, Zhou X, Lewis M G, Amara RR, Bosinger S, Silvestri G, Ertl H C J. Correlates of Protection Against SIV_{mac251} Infection in Rhesus Macaques Immunized With Chimpanzee-Derived Adenovirus Vectors. EBioMedicine. 2018 May; 31:25-35.
Cervasi B, Carnathan D G, Sheehan K M, Micci L, Paiardini M, Kurupati R, Tuyishime S, Zhou X Y, Else J G, Ratcliffe S J, Ertl H C, Silvestri G. Immunological and virological analyses of rhesus macaques immunized with chimpanzee adenoviruses expressing the simian immunodeficiency virus Gag/Tat fusion protein and challenged intrarectally with repeated low doses of SIVmac. J Virol. 2013 September; 87(17):9420-30.
Lasaro M O, Haut L H, Zhou X, Xiang Z, Zhou D, Li Y, Giles-Davis W, Li H, Engram J C, Dimenna L J, Bian A, Sazanovich M, Parzych E M, Kurupati R, Small J C, Wu T L, Leskowitz R M, Klatt N R, Brenchley J M, Garber D A, Lewis M, Ratcliffe S J, Betts M R, Silvestri G, Ertl H C. Vaccine-induced T cells provide partial protection against high-dose rectal SIVmac239 challenge of rhesus macaques. Mol Ther. 2011 February; 19(2):417-26.
Tatsis N, Lasaro M O, Lin S W, Haut L H, Xiang Z Q, Zhou D, Dimenna L, Li H, Bian A, Abdulla S, Li Y, Giles-Davis W, Engram J, Ratcliffe S J, Silvestri G, Ertl H C, Betts M R. Adenovirus vector-induced immune responses in nonhuman primates: responses to prime boost regimens. J Immunol. 2009 May 15; 182(10):6587-99.

### Other Embodiments

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations and subcombinations.

1. A composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter operably linked to a sequence encoding a heterologous protein, wherein the heterologous protein is at least one HIV protein selected from the group consisting of gp140 and Gag;
wherein gp140 is from a Chinese HIV clade selected from the group consisting of B, AE, BC and C; and
wherein Gag is from a Chinese HIV clade B.

2. The composition of claim 1, wherein the expression cassette is in the early gene E1 genomic region.

3. The composition of claim 1, wherein the expression cassette comprises a chimeric intron and/or CMV enhancer.

4. The composition of claim 1, wherein an early gene E3 genomic region consisting of ORF3, ORF4, ORF5, ORF6, and ORF7 is deleted.

5. The composition of claim 1, wherein the entire early gene E3 genomic region is deleted.

6. The composition of claim 1, wherein the promoter is a constitutive promoter.

7. The composition of claim 1, wherein the promoter is a cytomegalovirus immediate early promoter (CMV).

8. The composition of claim 1, wherein the nucleic acid sequence comprises SEQ ID NOs: 6 or 7.

9. A protein expression system comprising the composition of claim 1, wherein the nucleic acid sequence comprises SEQ ID NOs: 6 or 7.

10. A protein expression system comprising the composition of claim 1, wherein the heterologous protein encoded by the expression cassette comprises an amino acid sequence selected from the group consisting of SEQ ID NOs: 1-5.

11. A composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a constitutive promoter operably linked to a sequence encoding a heterologous protein, wherein the expression cassette is in the early gene E1 genomic region, wherein the heterologous protein is at least one HIV protein selected from the group consisting of gp140 and Gag;
wherein gp140 is from a Chinese HIV clade selected from the group consisting of B, AE, BC and C; and
wherein Gag is from a Chinese HIV clade B.

12. The composition of claim 11, wherein the nucleic acid sequence comprises SEQ ID NOs: 6 or 7.

13. A protein expression system comprising the composition of claim 11, wherein the heterologous protein encoded by the expression cassette comprises an amino acid sequence selected from the group consisting of SEQ ID NOs: 1-5.

14. A method of eliciting an immune response in a mammal against a heterologous protein, the method comprising administering to the mammal a composition comprising a nucleic acid sequence of a chimpanzee-derived adenovirus vector of serotype AdC6 or AdC7, wherein an early gene E1 genomic region is deleted, and wherein the nucleic acid sequence further comprises an expression cassette comprising a promoter operably linked to a sequence encoding a heterologous protein, wherein the heterologous protein is at least one HIV protein selected from the group consisting of gp140 and Gag;
wherein gp140 is from a Chinese HIV clade selected from the group consisting of B, AE, BC and C; and
wherein Gag is from a Chinese HIV clade B.

15. The method of claim 14, wherein the expression cassette is in the early gene E1 region.

16. The method of claim 14, wherein the expression cassette comprises a chimeric intron and/or CMV enhancer.

17. The method of claim 14, wherein an early gene E3 genomic region consisting of ORF3, ORF4, ORF5, ORF6, and ORF7 is deleted.

18. The composition of claim 14, wherein the entire early gene E3 genomic region is deleted.

19. The method of claim 14, wherein the promoter is a constitutive promoter.

20. The method of claim 14, wherein the promoter is a cytomegalovirus immediate early promoter (CMV).

21. The method of claim 14, wherein the nucleic acid sequence comprises SEQ ID NOs: 6 or 7.

22. A method of treating and/or preventing HIV in a mammal, the method comprising administering a therapeutically effective amount of a composition encoded by a nucleic acid sequence comprising SEQ ID NOs: 6 or 7.

23. A method of vaccinating a mammal against HIV infection, the method comprising administering to the mammal a therapeutically effective amount of the composition of claim 1, wherein administration of the composition elicits an immune response in the mammal.

24. The method of claim 23, wherein the composition is administered prophylactically to the mammal.

25. The method of claim 23, wherein the composition is administered therapeutically to the mammal.

26. The method of claim 23, wherein the composition is administered in combination with an adjuvant.

27. A method of generating an effector and memory T cell immune response to a heterologous protein in a mammal, the method comprising the steps of: (a) administering the composition of claim 1 to a mammal in an amount effective to elicit an immune response in the mammal; (b) administering a second effective amount of the composition of claim 1 at a second, subsequent time period, wherein T memory cells directed against the heterologous protein are reactivated in the mammal.

28. The method of claim 27, wherein the composition administered first in (a) and second in (b) comprises a same or a different HIV heterologous protein selected from the group consisting of gp140 and Gag.

29. The method of claim 27, wherein the composition administered first in (a) and second in (b) is a same or a different serotype selected from the group consisting of AdC6 and AdC7.

30. The method of claim 27, wherein the composition administered first in (a) and second in (b) is of a same or a different HIV Clade.

31. The method of claim 27, further comprising the step of administering an immunogen to the mammal.

32. The method of claim 31, wherein the immunogen comprises a heterologous protein, wherein the heterologous protein is at least one HIV protein selected from the group consisting of gp140 and Gag; wherein gp140 is from a Chinese HIV Clade selected from the group consisting of B, AE, BC and C; and wherein Gag is from a Chinese HIV clade B, wherein a B cell immune response is further augmented.

33. A method of generating an adaptive B cell immune response to a heterologous protein in a mammal, the method comprising the steps of: (a) administering the composition of claim 1 to a mammal in an amount effective to elicit an immune response in the mammal; (b) administering a second effective amount of the composition of claim 1 at a second, subsequent time period, wherein B memory cells directed against the heterologous protein are reactivated in the mammal.

34. The method of claim 33, wherein the composition administered first in (a) and second in (b) comprises a same or a different HIV heterologous protein selected from the group consisting of gp140 and Gag.

35. The method of claim 33, wherein the composition administered first in (a) and second in (b) has a same or a different serotype selected from the group consisting of AdC6 and AdC7.

36. The method of claim 33, wherein the composition administered first in (a) and second in (b) is of a same or a different HIV Clade.

37. The method of claim 33, further comprising the step of administering an immunogen to the mammal.

38. The method of claim 37, wherein the immunogen comprises a heterologous protein, wherein the heterologous protein is at least one HIV env protein selected from any Clade from any source, wherein the B cell immune response is further augmented.

39. The method of claim 14, wherein the mammal is a human.


SEQUENCE LISTING (7 sequences)

SEQ ID NO: 1 - protein, 643 aa, Artificial Sequence
  Expressed Protein
  Gp140 Clade AE1
MRVKGTQMNWPNLWKWGTLILGLVIMCSASDNLWVTVYYGVPVWRDANTTLFCASDAKAHETEVHNVWATYACVPTDPNPQEIPMENVTENFNMWKNNMVEQMQEDVISLWDQSLKPCVKLTPLCVTLICTNANLTKINSTNSGPKVIGNVTDEVRNCSFNMTTLLTDKKQKVYALFYKLDIVPIDNSNSSEYRLINCNTSVIKQACPKISFDPIPIHYCTPAGYAILKCNDKNFNGTGPCKNVSSVQCTHGIKPVVSTQLLLNGSLAEEEIIIRSENLTNNAKTIIVHLNKAVEINCTRPSNNTRTSIRIGPGQIFYRTGDIIGDIRQAYCEINGTKWNETLRQVAKKLKEQFNNTIKFQPPSGGDLEITMLHFNCRGEFFYCNTTKLFNSTWERNETIKGGNGNGNDTIILPCRIKQIINMWQGAGQAMYAPPISGIINCVSNITGILLTRDGGNTNETAEIFRPGGGNIKDNWRSELYKYKVVQIEPLGVAPTKAKLTVQARQLLSGIVQQQSNLLRAIEAQQHMLQLTVWGIKQLQARILAVESYLKHQQFLGLWGCSNKIICTTAVPWNSSWSNKSYDEIWENMTWIEWEREIGNYTNQIYDILTKSQEQQDKNEKELLELDQWASLWNWFSITKWLW

SEQ ID NO: 2 - protein, 639 aa, Artificial Sequence
  Expressed Protein
  Gp140 Clade B HM215399
MRVKGIRKNYQHLWRWGTMLLGMLMICSAAENLWVTVYYGVPVWKEATTTLFCASDAKAYDTEVHNIWATHACVPTDPNPQEVVLGNVTENFNMWKNDMVEQMHEDIISLWDQSLKPCVKLTPLCVTLNCTNLRNTNNTSSNTSNMTEGGEIKNCSFDITTSIRTKVKDYALFYELDIVAIDNTSYRLRQCNTSVITQACPKISFEPIPIHYCTPAGFAILKCNNKTFNGTGPCTNVSTVQCTHRIRPVVSTQLLLNGSLAEEEVVIRSSNFTDNAKVIIVQLKESVEINCTRPNNNTRKSIPLGPGKAWYTTGQIIGDIRQAHCNLSRAKWENTLQQITKKLREQFGNKTIIFNQSSGGDPEVVTHSFNCGGEFFYCNTSQLFNSTWYNNSTWNDTNDTTENSTITLPCRIKQIVNMWQEVGKAMYAPPIRGQIRCSSNITGLLLTRDGGKNESNTTETFRPGGGDMRDNWRSELYKYKVVKIEPLGVAPTRAKLTVQARQLLSGIVQQQRNLLRAIEAQQHLLQLTVWGIKQLQARVLAVERYLKDQQLLGIWGCSGKLICTTAVPWNVSWSNRSLSEIWDNMTWMEWEREIGNYTKQIYSLIEESQNQQEKNELELLEWDKWASLWNWFNITNWLW

SEQ ID NO: 3 - protein, 634 aa, Artificial Sequence
  Expressed Protein
  Gp140 Clade C
MRVRGTQRNYPQWWIWGILGFWMLMICNVGGNLWVTVYYGVPVWKEATTTLFCASDAKAYENEVHNVWATHACVPTDPNPQEMVLENVTENFNMWKNEMVNQMHEDVISLWDQSLKPCVKLTPLCVTLKCSNVTLKNNTVNSNETQYRKNCTFNTTTELKNRKQKVSAIFYRIDIVPLGNESSGNYRLINCNTSAITQACPKVSFDPIPIHYCTPAGYALLKCNNKTFNGTGPCNNVSTVQCTHGIKPVVSTQLLLNGSLAEEEIIIRSENLTNNVKTIIVHLNESVEIVCIRPGNNTRQSIRIGPGQTFYAPGEIIGNIRQAHCNINGTKWNETLQGVGKKLAEHFPNKTIKFKPSSGGDPEITTHSFNCRGEFFYCDTSGLFNSTYNSTYVPNGTESKPNITIQCRIKQIINMWQEVGRAMYAPPIKGSITCKSNITGLLLVRDGGANTTEEIFRPGGGDMRDNWRSELYKYKVVEIKPLGIAPTEAKLTVQARQLLSGIVQQQNNLLKAIEAQQHMLQLTVWGIKQLQTRVLAIERYLKDQQLLGIWGCSGKLICTTAVPWNSSWSNKTQDEIWKNMTWMQWDREINNYTNTIYSLLEESQNQQEKNEKDLLALDSWKNLWNWFDISNWLW

SEQ ID NO: 4 - protein, 647 aa, Artificial Sequence
  Expressed Protein
  Gp140 Clade BC
MRVMGIRRNCQHLWRWGIMLLGMLMICSVVGNLWVTVYYGVPVWKEATTTLFCASDAKAYDTEVHNVWATHACVPTDPNPQEMVLENVTENFNMWKNEMVNQMQEDVISLWDQSLKPCVKLTPLCVTLKCKNVSSNSTETPKLRGNSSETYKDEEMKNCSFNATTILRDKKQEVYALFYKLDIAPLLLNSSENSSAYYSLINCNTSAITQACPKVSFDPIPIHYCTPAGYAILKCNDKKFNGTGPCSNVSTVQCTHGIKPVVSTQLLLNGSLAEGEVIIRSKNLTDNAKTIIVQLNRSVEIVCTRPNNNTRKSIRIGPGQTFYATGDIIGDIRQAHCNISEDMWNETLHWVSRKLAEHFPNRTINFTSSSGGDLEIATHSFNCRGEFFYCNTSRLFNGTYMFNGTRGNSSSNSTITIPCRIKQIINMWQQVGRAMYAPPIEGNLTCRSNITGLLLVRDGGDNTNKTEIFRPQGGDMRDNWRSELYKYKVVEIKPLGIAPTTAKLTVQARQLLSGIVQQQSNLLRAIEAQQHLLQLTVWGIKQLQTRVLAIERYLKDQQLLGIWGCSGKLICTTAVPWNSSWSNKTQDEIWNNLTWMQWDKEISNYTDTIYKLLEDSQNQQERNEKDLLALDSWKNLWSWFDITNWLW

SEQ ID NO: 5 - protein, 500 aa, Artificial Sequence
  Expressed Protein
  Gp140 Clade B JF932500
MGARASVLSGGELDRWEKIRLRPGGKKKYRLKHVVWASRELERFAVNPGLLETSEGCRQILEQLQPSLQTGSEELRSLYNTIAVLYCVHQKIEIKDTKEALDKIEEEQNKSKKKAQQAAADTGNNSQVSQNYPIVRNLQGQMVHQPLSPRTLNAWVKVVEEKAFSPEVIPMFSALSEGATPQDLNTMLNTVGGHQAAMQMLKETINEEAAEWDRLHPPQAGPIAPGQIREPRGSDIAGTTSNLQEQIAWMTNNPPIPVGEIYKRWIILGLNKIVRMYSPTSILDIKQGPKEPFRDYVDRFYKTLRAEQASQDVKNWMTETLLVQNANPDCKTILKALGPAATLEEMMTACQGVGGPSHKARILAEAMSQVTNSASVMMQRGNFRNQRKPVKCFNCGKEGHIAKNCRAPRKKGCWKCGKEGHQMKDCTERQANFLGKIWPSHKGRPGNFLQSRPEPTAPPEESFRFGEETTTPSQKQEQIDKELYPLASLKSLFGNDPSSQ

SEQ ID NO: 6 - DNA, 34406 bp, Artificial Sequence
  Vector
  C6 020 CMV-HIV gp140 AE1
catcatcaataatatacctcaaacttttggtgcgcgttaatatgcaaatgagctgtttgaatttggggagggaggaaggtgattggctgcgggagcggcgaccgttaggggcggggcgggtgacgttttgatgacgtggctatgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtacgatatcatttccccgaaagtgccacctgaccgtaactataacggtcctaaggtagcgaaagctcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaaggctagagtacttaatacgactcactataggctagcatgagagtgaaggggacacagatgaattggccaaacttgtggaaatgggggactttgatccttgggttggtgatcatgtgtagtgcctcagacaacttgtgggttacagtttattatggagttcctgtgtggagagatgcaaataccaccctattttgtgcatcagatgccaaagcacatgagacagaagtgcacaatgtctgggccacatatgcctgtgtacccacagatcccaacccacaagaaatacccatggaaaatgtgacagaaaattttaacatgtggaaaaataacatggtagagcaaatgcaggaggatgtaatcagtttatgggatcaaagtctaaagccatgtgtaaagttaactcctctctgcgttactttaatttgtaccaatgctaacttgaccaagatcaacagtaccaatagcgggcctaaagtaataggaaatgtaacagatgaagtaagaaactgttcttttaatatgaccacattactaacagataagaagcaaaaggtttatgcacttttttataagcttgatatagtaccaattgataatagtaatagtagtgagtatagattaataaattgtaatacttcagtcattaagcaggcttgtccaaagatatcctttgatccaattcctatacattattgtactccagctggttatgcgattttaaaatgtaatgataagaatttcaatgggacagggccatgtaaaaatgtcagctcagtacagtgcacacatggaattaagccagtggtctcaactcaattactgttaaatggcagtctagcagaagaagagataataatcagatctgaaaatctcacaaacaatgccaaaaccataatagtgcaccttaataaggctgtagaaatcaattgtaccagaccctccaacaatacaagaacaagtataagaataggaccaggacaaatattttatagaacaggagacataataggagatataagacaagcatattgtgaaattaatggaacaaaatggaatgaaactttaagacaggtagcaaaaaaattaaaagagcaatttaataacacaataaaattccagccaccctcaggaggagatctagaaattacaatgcttcattttaattgtagaggggaatttttctattgcaatacaacaaaactgttcaatagtacttgggaaagaaatgagaccataaaagggggtaatggcaatggcaatgacactatcatacttccatgcaggataaagcaaatcataaacatgtggcaaggagcaggacaagcaatgtatgctcctcccatcagtggaataattaactgtgtatcaaatattacaggaatactattgacaagagatggtggtaatactaatgaaactgccgagatcttcagacctggaggaggaaatataaaggacaattggagaagtgaattatataaatataaagtagtacaaattgaaccactaggagtagcacccaccaaggcaaagctgacggtacaggccagacaattattgtctggtatagtgcaacagcaaagcaatttgctgagggctatagaggcgcagcagcatatgttgcaactcacagtctggggcattaaacagctccaggcaagaatcctggctgtggaaagctacctaaagcatcaacagttcctaggactttggggctgctctaacaaaattatctgcaccactgctgtaccctggaattcctcttggagtaataaatcttatgatgagatttgggaaaatatgacatggatagaatgggagagagaaattggcaattacacaaaccaaatatatgatatacttacaaaatcgcaggaacagcaggacaaaaatgaaaaggaactgttggaattggatcaatgggcaagtctgtggaattggtttagcataacaaaatggctgtggtaatgtacaagtaaagcggccgccactgtgctggatgatccgagctcggtacctctagagtcgacccgggcggccaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcagatctgcagatctgaattcatctatgtcgggtgcggagaaagaggtaatgaaatggcattatgggtattatgggtctgcattaatgaatcggtcagatatcgacatatgctggccaccgtgcatgtggcctcgcacccccgcaagacatggcccgagttcgagcacaacgtcatgacccgctgcaatgtgcacctgggctcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttggggctgggtgtgagcctgcatgaggggcagaatgactaaaatctgtggttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgagttccaccaataatcccgccagcctgaacgaggagaagctgctgctgctgatggcccagctcgaggccctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggatcttttccaggacccggtagaggtgggcttggatgttgaggtacatgggcatgagcccgtcccgggggtggaggtagctccattgcagggcctcgtgctcggggatggtgttgtaaatcacccagtcatagcaggggcgcagggcgtggtgctgcacgatgtccttgaggaggagactgatggccacgggcagccccttggtgtaggtgttgacgaacctgttgagctgggagggatgcatgcggggggagatgagatgcatcttggcctggatcttgagattggcgatgttcccgcccagatcccgccgggggttcatgttgtgcaggaccaccagcacggtgtatccggtgcacttggggaatttgtcatgcaacttggaagggaaggcgtgaaagaatttggagacgcccttgtgaccgcccaggttttccatgcactcatccatgatgatggcgatgggcccgtgggcggcggcctgggcaaagacgtttcgggggtcggacacatcgtagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcaaccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagtccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccagggccgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcggggcggttggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggcgggcatgacctcggcactcaggttgtcagtttctagaaacgaggaggatttgatattgacggtgccgttggagacgcctttcatgagcccctcgtccatttggtcagaaaagacgatctttttgttgtcgagcttggtggcgaaggagccgtagagggcgttggagagcagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcaggagctcggggtcgaagtagctgatgcaggtgcccagattgtccagcgccgcttgccagtcgcgcacggccagcgcgcgctcgtaggggctgaggggcgtgccccagggcatggggtgcgtgagcgcggaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgtgccgaggttggagcgttgcggcttttcggcgcggtagacgatctggcggaagatggcgtgggagttggaggagatggtgggcctttggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcatacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgcgcggccttgcgcagggaggtgtgggtgagggcgaaggtgtcgcgcaccatgaccttgaggaactggtgcttgaagtcgaggtcgtcgcagccgccctgctcccagagttggaagtccgtgcgcttcttgtaggcggggttaggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcggcccttgacgtggggcagcttcttgagctcgtcgtaggtgagctcggcggggtcgctgagcccgtgctgctcgagggcccagtcggcgacgtgggggttggcgctgaggaaggaagtccagagatccacggccagggcggtctgcaagcggtcccggtactgacggaactgttggcccacggccattttttcgggggtgacgcagtagaaggtgcgggggtcgccgtgccagcggtcccacttgagctggagggcgaggtcgtgggcgagctcgacgagcggcgggtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttggcgaggaatttcagtgggcagtggagcgctggcggctgcatctcgtgctgtactacgtcttggccatcggcgtggccatcgtctgcctcgatggtggtcatgctgacgagcccgcgcgggaggcaggtccagacctcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggctacgtccacggcttgcagggtgccgtgcccctggggcgccaccaccgtgccccgtttcttcttgggcgctgcttccatgtcggtcagaagcggcggcgaggacgcgcgccgggcggcaggggcggctcggggcccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctcgacggtggccgcgaggtcgttggagatgcggcccatgagctgcgagaaggcgttcatgccggcctcgttccagacgcggctgtagaccacggctccgtcggggtcgcgcgcgcgcatgaccacctgggcgaggttgagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgttccatggcctcgtagaagtccacggcgaagttgaaaaactgggagttgcgcgccgagacggtcaactcctcctccagaagacggatgagctcggcgatggtggcgcgcacctcgcgctcgaaggccccggggggctcctcttccatctcctcctcttcctcctccactaacatctcttctacttcctcctcaggaggcggtggcgggggaggggccctgcgtcgccggcggcgcacgggcagacggtcgatgaagcgctcgatggtctccccgcgccggcgacgcatggtctcggtgacggcgcgcccgtcctcgcggggccgcagcatgaagacgccgccgcgcatctccaggtggccgccgggggggtctccgttgggcagggagagggcgctgacgatgcatcttatcaattgacccgtagggactccgcgcaaggacctgagcgtctcgagatccacgggatccgaaaaccgctgaacgaaggcttcgagccagtcgcagtcgcaaggtaggctgagcccggtttcttgttcttcgggtatttggtcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctccacgggcacctcctcctcgcccgcgcggccgtgcatgcgcgtgagcccgaacccgcgctgcggctggacgagcgccaggtcggcgacgacgcgctcggtgaggatggcctgctggatctgggtgagggtggtctggaagtcgtcgaagtcgacgaagcggtggtaggctccggtgttgatggtgtaggagcagttggccatgacggaccagttgacggtctggtggccgggtcgcacgagctcgtggtacttgaggcgcgagtaggcgcgcgtgtcgaagatgtagtcgttgcaggcgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgctaacgaaacctccaggatacggaggcgggtcgttttttggccttggtcgctggtcatgaaaaactagtaagcgcggaaagcggccgcccgcgatggctcgctgccgtagtctggagaaagaatcgccagggttgcgttgcggtgtgccccggttcgagcctcagcgctcggcgccggccggattccgcggctaacgtgggcgtggctgccccgtcgtttccaagaccccttagccagccgacttctccagttacggagcgagcccctctttttttttcttgtgtttttgccagatgcatcccgtactgcggcagatgcgcccccaccctccaccacaaccgcccctaccgcagcagcagcaacagccggcgcttctgcccccgccccagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattttgcagagcatcgtggtgcaggagcgcgggctgccgctgtccgagaagctggcggccatcaacttctcggtgctgagtctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcccagccgccgggccttggaagctgccggcggttccccctacgtggaggaggtggacgatgaggaggaggagggcgagtacctggaagactgatggcgcgaccgtatttttgctagatgcagcaacagccaccgccgccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggtgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcggacaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcctgatgcaggagggggccacgcccagcgcggcgctcgacatgaccgcgcgcaacatggagcccagcatgtacgcccgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggacgacgtggacagcagcgtgttctcgccgcgtccaggaaccaatgccgtgtggaagaaagagggcggggaccggcggccgtcctcggcgctgtccggtcgcgcgggtgctgccgcggcggtgcccgaggccgccagccccttcccgagcctgcccttttcgctgaacagcgtgcgcagcagcgagctgggtcggctgacgcgaccgcgcctgctgggcgaggaggagtacctgaacgactccttgttgaggcccgagcgcgagaagaacttccccaataacgggatagagagcctggtggacaagatgagccgctggaagacgtacgcgcacgagcacagggacgagccccgagctagcagcgcaggcacccgtagacgccagcggcacgacaggcagcggggactggtgtgggacgatgaggattccgccgacgacagcagcgtgttggacttgggtgggagtggtggtaacccgttcgctcacctgcgcccccgtatcgggcgcctgatgtaagaatctgaaaaaataaaagacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcagacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaacggggtggatgatgattatgatggtagtcaggacgagctgacctacgagtgggtggagtttgagctgcccgagggcaacttctcggtgaccatgaccatcgatctgatgaacaacgccatcatcgacaactacttggcggtggggcggcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctcttggatgtcgaagcctacgagaaaagcaaggaggatagcaccgccgcggcgaccgcagccgtggccaccgcctctaccgaggtgcggggcgataattttgctagcgctgcggcagcggccgaggcggctgaaaccgaaagtaagatagtcatccagccggtggagaaggacagcaaggacaggagctacaacgtgctcgcggacaagaaaaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcgctcgcgcaccaccgtcgacgacgtgatcgaccaggtggtggccgacgcgcgcaactacacgcccgccgccgcgcccgtctccaccgtggacgccgtcatcgacagcgtggtggccgacgcgcgccggtacgcccgcaccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggcggcggtgaaggaggaaagaaagccccgcaaactgaagcgggtcaaaaaggacaaaaaggaggaggaagatgacggactggtggagtttgtgcgcgagttcgccccccggcggcgcgtgcagtggcgcgggcggaaagtgaaaccggtgctgcggcccggcaccacggtggtcttcacgcccggcgagcgttccggctccgcctccaagcgctcctacgacgaggtgtacggggacgaggacatcctcgagcaggcggtcgagcgtctgggcgagtttgcgtacggcaagcgcagccgccccgcgcccttgaaagaggaggcggtgtccatcccgctggaccacggcaaccccacgccgagcctgaagccggtgaccctgcagcaggtgctaccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccagcaccagcttccaccagcactcgccgaagacgcaagtacggcgcggccagcctgctgatgcccaactacgcgctgcatccttccatcatccccacgccgggctaccgcggcacgcgcttctaccgcggctacaccagcagccgccgccgcaagaccaccacccgccgccgtcgtcgcagccgccgcagcagcaccgcgacttccgccttggtgcggagagtgtatcgcagcgggcgcgagcctctgaccctgccgcgcgcgcgctaccacccgagcatcgccatttaactaccgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacaaaaaagcatggatttgtaataaaaaaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagaacagaacttccagcagaaggtggttgatggcctggcctcaggcatcaacggggtggttgacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggcctgcccaccacgcggcccgtggcgcctctggccaccggagtgctgaaacccagcagcagccagcccgcgaccctggacttgcctccgcctcgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtatatgtatgtccgccgaccagaaggaggagtgtgaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggaccggggccctagcttcaaaccctactctggcaccgcctacaacagcctagctcccaagggagctcccaattccagccagtgggagcaagcaaaaacaggcaatgggggaactatggaaacacacacatatggtgtggccccaatgggcggagagaatattacaaaagatggtcttcaaattggaactgacgttacagcgaatcagaataaaccaatttatgccgacaaaacatttcaaccagaaccgcaagtaggagaagaaaattggcaagaaactgaaaacttttatggcggtagagctcttaaaaaagacacaaacatgaaaccttgctatggctcctatgctagacccaccaatgaaaaaggaggtcaagctaaacttaaagttggagatgatggagttccaaccaaagaattcgacatagacctggctttctttgatactcccggtggcaccgtgaacggtcaagacgagtataaagcagacattgtcatgtataccgaaaacacgtatttggaaactccagacacgcatgtggtatacaaaccaggcaaggatgatgcaagttctgaaattaacctggttcagcagtctatgcccaacagacccaactacattgggttcagggacaactttatcggtcttatgtactacaacagcactggcaatatgggtgtgcttgctggtcaggcctcccagctgaatgctgtggttgatttgcaagacagaaacaccgagctgtcctaccagctcttgcttgactctttgggtgacagaacccggtatttcagtatgtggaaccaggcggtggacagttatgaccccgatgtgcgcatcatcgaaaaccatggtgtggaggatgaattgccaaactattgcttccccttggacggctctggcactaacgccgcataccaaggtgtgaaagtaaaagatggtcaagatggtgatgttgagagtgaatgggaaaatgacgatactgttgcagctcgaaatcaattatgtaaaggtaacattttcgccatggagattaatctccaggctaacctgtggagaagtttcctctactcgaacgtggccctgtacctgcccgactcctacaagtacacgccgaccaacgtcacgctgccgaccaacaccaacacctacgattacatgaatggcagagtgacacctccctcgctggtagacgcctacctcaacatcggggcgcgctggtcgctggaccccatggacaacgtcaaccccttcaaccaccaccgcaacgcgggcctgcgctaccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctaggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggatggtccttcacgcgcctgaagacccgcgagacgccctcgctcggctccgggttcgacccctacttcgtctactcgggctccatcccctacctagacggcaccttctacctcaaccacaccttcaagaaggtctccatcaccttcgactcctccgtcagctggcccggcaacgaccgcctcctgacgcccaacgagttcgaaatcaagcgcaccgtcgacggagagggatacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaagccgtctacctgcgcacgcccttctcggccggcaacgccaccacctaagccgctcttgcttcttgcaagatgacggcgggctccggcgagcaggagctcagggccatcctccgcgacctgggctgcgggccctgcttcctgggcaccttcgacaagcgcttccctggattcatggccccgcacaagctggcctgcgccatcgtgaacacggccggccgcgagaccgggggcgagcactggctggccttcgcctggaacccgcgctcccacacatgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttactgacgggggtgcccaacggcatgctccagtcgccccaggtggaacccaccctgcgccgcaaccaggaagcgctctaccgcttcctcaatgcccactccgcctactttcgctcccaccgcgcgcgcatcgagaaggccaccgccttcgaccgcatgaatcaagacatgtaaaaaaccggtgtgtgtatgtgaatgctttattcataataaacagcacatgtttatgccaccttctctgaggctctgactttatttagaaatcgaaggggttctgccggctctcggcatggcccgcgggcagggatacgttgcggaactggtacttgggcagccacttgaactcggggatcagcagcttgggcacggggaggtcggggaacgagtcgctccacagcttgcgcgtgagttgcagggcgcccagcaggtcgggcgcggagatcttgaaatcgcagttgggacccgcgttctgcgcgcgagagttgcggtacacggggttgcagcactggaacaccatcagggccgggtgcttcacgcttgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagccggcgtcgtgcacgcagcagcgcgcgtcgttgttggccagctgcaccacgctgcgcccccagcggttctgggtgatcttggcccggttggggttctccttcagcgcgcgctgcccgttctcgctcgccacatccatctcgatagtgtgctccttctggatcatcacggtcccgtgcaggcaccgcagcttgccctcggcttcggtgcagccgtgcagccacagcgcgcagccggtgcactcccagttcttgtgggcgatctgggagtgcgagtgcacgaagccctgcaggaagcggcccatcatcgcggtcagggtcttgttgctggtgaaggtcagcgggatgccgcggtgctcctcgttcacatacaggtggcagatgcggcggtacacctcgccctgctcgggcatcagctggaaggcggacttcaggtcgctctccacgcggtaccggtccatcagcagcgtcatcacttccatgcccttctcccaggccgaaacgatcggcaggctcagggggttcttcaccgccattgtcatcttagtcgccgccgccgaggtcagggggtcgttctcgtccagggtctcaaacactcgcttgccgtccttctcgatgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcgatgtgctgggagagcgcgagttctcgttcaccacgactatttcttcttcttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaacaacaagcatggagactcagccatcgtcgccaacatcgccatctgcccccgccgccaccgccgacgagaaccagcagcagaatgaaagcttaaccgccccgccgcccagccccacctccgacgccgcggccccagacatgcaagagatggaggaatccatcgagattgacctgggctacgtgacgcccgcggagcacgaggaggagctggcagcgcgcttttcagccccggaagagaaccaccaagagcagccagagcaggaagcagagaacgagcagaaccaggctgggcacgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgtgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcacctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtcctcaagcgcacggtcgagctgacccacttcgcctacccggcgctcaacctgccccccaaggtcatgagcgccgtcatggaccaggtgctcatcaagcgcgcctcgcccctctcggaggaggagatgcaggaccccgagagttcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttctttgccgacgcggagaccctgcgcaaggtcgaggagaacctgcactacctcttcaggcacgggttcgtgcgccaggcctgcaagatctccaacgtggagctgaccaacctggtctcctacatgggcatcctgcacgagaaccgcctggggcaaaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgtaccaccgcctcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcgctgccctcggacttcgtgccgctgaccttccgcgagtgccccccgccgctctggagccactgctacttgctgcgcctggccaactacctggcctaccactcggacgtgatcgaggacgtcagcggcgagggtctgctggagtgccactgccgctgcaacctctgcacgccgcaccgctccctggcctgcaacccccagctgctgagcgagacccagatcatcggcaccttcgagttgcaaggccccggcgacggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacttggacccccagaccggagaggagctcaaccccagcttcccccaggatgccccgaggaagcagcaagaagctgaaagtggagctgccgccgccggaggatttggaggaagactgggagagcagtcaggcagaggaggaggagatggaagactgggacagcactcaggcagaggaggacagcctgcaagacagtctggaggaggaagacgaggtggaggaggcagaggaagaagcagccgccgccagaccgtcgtcctcggcggagaaagcaagcagcacggataccatctccgctccgggtcggggtcgcggcggccgggcccacagtaggtgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagaaaaccagcggcagcagcagctagaaaatccacagcggcggcaggtggactgaggatcgcggcgaacgagccggcgcagacccgggagctgaggaaccggatctttcccaccctctatgccatcttccagcagagtcgggggcaggagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggcggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcagctgacctagctcggcttcgacacctggaccactgccgccgcttccgctgcttcgctcgggatctcgccgagtttgcctactttgagctgcccgaggagcaccctcagggcccagcccacggagtgcggatcatcgtcgaagggggcctcgactcccacctgcttcggatcttcagccagcgaccgatcctggtcgagcgcgaacaaggacagacccttcttactttgtactgcatctgcaaccaccccggcctgcatgaaagtctttgttgtctgctgtgtactgagtataataaaagctgagatcagcgactactccggactcgattgtggtgttcctgctatcaaccggtccctgttcttcaccgggaacgagaccgagctccagctccagtgtaagccccacaagaagtacctcacctggctgttccagggctccccgatcgccgttgtcaaccactgcgacaacgacggagtcctgctgagcggccctgccaaccttactttttccacccgcagaagcaagctccagctcttccaacccttcctccccgggacctatcagtgcgtctcaggaccctgccatcacaccttccacctgatcccgaataccacagcgccgctccccgctactaacaaccaaactacccaccaacgccaccgtcgcgacctttcctctgaatctaataccactaccggaggtggcttctgctgttagtgctcccccgtcccgtcgacccccggtcccccactcagtcccccgaggaggttcgcaaatgcaaattccaagaaccctggaaattcctcaaatgctaccgccaaaaatcagacatgcatcccagctggatcatgatcattgggatcgtgaacattctggcctgcaccctcatctcctttgtgatttacccctgctttgactttggttggaactcgccagaggcgctctatctcccgcctgaacctgacacaccaccacagcagcaacctcaggcacacgcactaccaccaccacagcctaggccacaatacatgcccatattagactatgaggccgagccacagcgacccatgctccccgctattagttacttcaatctaaccggcggagatgactgacccactggccaataacaacgtcaacgaccttctcctggacatggacggccgcgcctcggagcagcgactcgcccaacttcgcattcgtcagcagcaggagagagccgtcaaggagctgcaggacggcatagccatccaccagtgcaagagaggcatcttctgcctggtgaaacaggccaagatctcctacgaggtcacccagaccgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaagatcatattgatgatgatttaaataaaaaaaataatcatttgatttgaaataaagatacaatcatattgatgatttgagtttaacaaaaataaagaatcacttacttgaaatctgataccaggtctctgtccatgttttctgccaacaccacctcactcccctcttcccagctctggtactgcaggccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgttgtccctgcgactggctgaccccgtcaccaccaagaacggggaaatcaccctcaagctgggagagggggtggacctcgactcgtcgggaaaactcatctccaacacggccaccaaggccgccgcccctctcagtatttcaaacaacaccatttcccttaaaactgctgcccctttctacaacaacaatggaactttaagcctcaatgtctccacaccattagcagtatttcccacatttaacactttaggcataagtcttggaaacggtcttcagacttcaaataagttgttgactgtacaactaactcatcctcttacattcagctcaaatagcatcacagtaaaaacagacaaagggctatatattaactccagtggaaacagaggacttgaggctaatataagcctaaaaagaggactagtttttgacggtaatgctattgcaacatatattggaaatggcttagactatggatcttatgatagtgatggaaaaacaagacccgtaattaccaaaattggagcaggattaaattttgatgctaacaaagcaatagctgtcaaactaggcacaggtttaagttttgactccgctggtgccttgacagctggaaacaaacaggatgacaagctaacactttggactacccctgacccaagccctaattgtcaattactttcagacagagatgccaaatttactctctgtcttacaaaatgcggtagtcaaatactaggcactgtggcagtggcggctgttactgtaggatcagcactaaatccaattaatgacacagtcaaaagcgccatagttttccttagatttgattccgatggtgtactcatgtcaaactcatcaatggtaggtgattactggaactttagggagggacagaccactcaaagtgtagcctatacaaatgctgtgggattcatgccaaatataggtgcatatccaaaaacccaaagtaaaacacctaaaaatagcatagtcagtcaggtatatttaactggagaaactactatgccaatgacactaaccataactttcaatggcactgatgaaaaagacacaaccccagttagcacctactctatgacttttacatggcagtggactggagactataaggacaaaaatattacctttgctaccaactcattctctttttcctacatcgcccaggaataatcccacccagcaagccaaccccttttcccaccacctttgtctatatggaaactctgaaacagaaaaataaagttcaagtgttttattgaatcaacagttttacaggactcgagcagttatttttcctccaccctcccaggacatggaatacaccaccctctccccccgcacagccttgaacatctgaatgccattggtgatggacatgcttttggtctccacgttccacacagtttcagagcgagccagtctcggatcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggttcgggtccagggactccctcagcatgatgcccacggccctcagcatcagtcgtctggtgcggcgggcgcagcagcgcatgcgaatctcgctcaggtcactgcagtacgtgcaacacaggaccaccaggttgttcaacagtccatagttcaacacgctccagccgaaactcatcgcgggaaggatgctacccacgtggccgtcgtaccagatcctcaggtaaatcaagtggcgctccctccagaagacgctgcccatgtacatgatctccttgggcatgtggcggttcaccacctcccggtaccacatcaccctctggttgaacatgcagccccggatgatcctgcggaaccacagggccagcaccgccccgcccgccatgcagcgaagagaccccggatcccggcaatgacaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttctcgaccgtgtcatgatgcagttgctttcggacattttcgtacttgctgtagcagaacctggtccgggcgctgcacaccgatcgccggcggcggtctcggcgcttggaacgctcggtgttaaagttgtaaaacagccactctctcagaccgtgcagcagatctagggcctcaggagtgatgaagatcccatcatgcctgatagctctgatcacatcgaccaccgtggaatgggccaggcccagccagatgatgcaattttgttgggtttcggtgacggcgggggagggaagaacaggaagaaccatgattaacttttaatccaaacggtctcggagcacttcaaaatgaaggtcacggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaaccatcatgttacactcctgcaccatccccagataattttcatttttccagccttgaatgattcgaactagttcctgaggtaaatccaagccagccatgataaaaagctcgcgcagagcaccctccaccggcattcttaagcacaccctcataattccaagatattctgctcctggttcacctgcagcagattgacaagcggaatatcaaaatctctgccgcgatccctgagctcctccctcagcaataactgtaagtactctttcatatcgtctccgaaatttttagccataggacccccaggaataagagaagggcaagccacattacagataaaccgaagtcccccccagtgagcattgccaaatgtaagattgaaataagcatgctggctagacccggtgatatcttccagataactggacagaaaatcgggtaagcaatttttaagaaaatcaacaaaagaaaaatcttccaggtgcacgtttagggcctcgggaacaacgatggagtaagtgcaaggggtgcgttccagcatggttagttagctgatctgtaaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgagaagaagcatacacccccggaacattggagtccgtgagtgaaaaaaagcggccgaggaagcaatgaggcactacaacgctcactctcaagtccagcaaagcgatgccatgcggatgaagcacaaaattttcaggtgcgtaaaaaatgtaattactcccctcctgcacaggcagcgaagctcccgatccctccagatacacatacaaagcctcagcgtccatagcttaccgagcggcagcagcagcggcacacaacaggcgcaagagtcagagaaaagactgagctctaacctgtccgcccgctctctgctcaatatatagccccagatctacactgacgtaaaggccaaagtctaaaaatacccgccaaataatcacacacgcccagcacacgcccagaaaccggtgacacactcagaaaaatacgcgcacttcctcaaacggccaaactgccgtcatttccgggttcccacgctacgtcatcaaaacacgactttcaaattccgtcgaccgttaaaaacatcacccgccccgcccctaacggtcgccgctcccgcagccaatcaccttcctccctccccaaattcaaacagctcatttgcatattaacgcgcaccaaaagtttgaggtatattattgatgatg

SEQ ID NO: 7 - DNA, 34058 bp, Artificial Sequence
  Vector
  C7 010 CMV-HIV gp140 AE1
catcatcaataatatacctcaaacttttggtgcgcgttaatatgcaaatgagctgtttgaatttggggagggaggaaggtgattggccgagagacgggcgaccgttaggggcggggcgggtgacgttttgatgacgtggccgtgaggcggagccggtttgcaagttctcgtgggaaaagtgacgtcaaacgaggtgtggtttgaacacggaaatactcaattttcccgcgctctctgacaggaaatgaggtgtttctgggcggatgcaagtgaaaacgggccattttcgcgcgaaaactgaatgaggaagtgaaaatctgagtaatttcgcgtttatggcagggaggagtatttgccgagggccgagtagactttgaccgattacgtgggggtttcgattaccgtatttttcacctaaatttccgcgtacggtgtcaaagtccggtgtttttacgtacgatatcatttccccgaaagtgccacctgaccgtaactataacggtcctaaggtagcgaaagctcagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcactagaagctttattgcggtagtttatcacagttaaattgctaacgcagtcagtgcttctgacacaacagtctcgaacttaagctgcagaagttggtcgtgaggcactgggcaggtaagtatcaaggttacaagacaggtttaaggagaccaatagaaactgggcttgtcgagacagagaagactcttgcgtttctgataggcacctattggtcttactgacatccactttgcctttctctccacaggtgtccactcccagttcaattacagctcttaaaaggctagagtacttaatacgactcactataggctagcatgagagtgaaggggacacagatgaattggccaaacttgtggaaatgggggactttgatccttgggttggtgatcatgtgtagtgcctcagacaacttgtgggttacagtttattatggagttcctgtgtggagagatgcaaataccaccctattttgtgcatcagatgccaaagcacatgagacagaagtgcacaatgtctgggccacatatgcctgtgtacccacagatcccaacccacaagaaatacccatggaaaatgtgacagaaaattttaacatgtggaaaaataacatggtagagcaaatgcaggaggatgtaatcagtttatgggatcaaagtctaaagccatgtgtaaagttaactcctctctgcgttactttaatttgtaccaatgctaacttgaccaagatcaacagtaccaatagcgggcctaaagtaataggaaatgtaacagatgaagtaagaaactgttcttttaatatgaccacattactaacagataagaagcaaaaggtttatgcacttttttataagcttgatatagtaccaattgataatagtaatagtagtgagtatagattaataaattgtaatacttcagtcattaagcaggcttgtccaaagatatcctttgatccaattcctatacattattgtactccagctggttatgcgattttaaaatgtaatgataagaatttcaatgggacagggccatgtaaaaatgtcagctcagtacagtgcacacatggaattaagccagtggtctcaactcaattactgttaaatggcagtctagcagaagaagagataataatcagatctgaaaatctcacaaacaatgccaaaaccataatagtgcaccttaataaggctgtagaaatcaattgtaccagaccctccaacaatacaagaacaagtataagaataggaccaggacaaatattttatagaacaggagacataataggagatataagacaagcatattgtgaaattaatggaacaaaatggaatgaaactttaagacaggtagcaaaaaaattaaaagagcaatttaataacacaataaaattccagccaccctcaggaggagatctagaaattacaatgcttcattttaattgtagaggggaatttttctattgcaatacaacaaaactgttcaatagtacttgggaaagaaatgagaccataaaagggggtaatggcaatggcaatgacactatcatacttccatgcaggataaagcaaatcataaacatgtggcaaggagcaggacaagcaatgtatgctcctcccatcagtggaataattaactgtgtatcaaatattacaggaatactattgacaagagatggtggtaatactaatgaaactgccgagatcttcagacctggaggaggaaatataaaggacaattggagaagtgaattatataaatataaagtagtacaaattgaaccactaggagtagcacccaccaaggcaaagctgacggtacaggccagacaattattgtctggtatagtgcaacagcaaagcaatttgctgagggctatagaggcgcagcagcatatgttgcaactcacagtctggggcattaaacagctccaggcaagaatcctggctgtggaaagctacctaaagcatcaacagttcctaggactttggggctgctctaacaaaattatctgcaccactgctgtaccctggaattcctcttggagtaataaatcttatgatgagatttgggaaaatatgacatggatagaatgggagagagaaattggcaattacacaaaccaaatatatgatatacttacaaaatcgcaggaacagcaggacaaaaatgaaaaggaactgttggaattggatcaatgggcaagtctgtggaattggtttagcataacaaaatggctgtggtaatgtacaagtaaagcggccgccactgtgctggatgatccgagctcggtacctctagagtcgacccgggcggccaaaccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagcagatctgcagatctgaattcatctatgtcgggtgcggagaaagaggtaatgaaatggcattatgggtattatgggtctgcattaatgaatcggccagatatcgatatgctggccaccgtgcatgtgacctcgcacccccgcaagacatggcccgagttcgagcacaacgtcatgacccgatgcaatgtgcacctggggtcccgccgaggcatgttcatgccctaccagtgcaacatgcaatttgtgaaggtgctgctggagcccgatgccatgtccagagtgagcctgacgggggtgtttgacatgaatgtggagctgtggaaaattctgagatatgatgaatccaagaccaggtgccgggcctgcgaatgcggaggcaagcacgccaggcttcagcccgtgtgtgtggaggtgacggaggacctgcgacccgatcatttggtgttgtcctgcaacgggacggagttcggctccagcggggaagaatctgactagagtgagtagtgtttgggggaggtggagggcttgtatgaggggcagaatgactaaaatctgtgtttttctgtgtgttgcagcagcatgagcggaagcgcctcctttgagggaggggtattcagcccttatctgacggggcgtctcccctcctgggcgggagtgcgtcagaatgtgatgggatccacggtggacggccggcccgtgcagcccgcgaactcttcaaccctgacctacgcgaccctgagctcctcgtccgtggacgcagctgccgccgcagctgctgcttccgccgccagcgccgtgcgcggaatggccctgggcgccggctactacagctctctggtggccaactcgacttccaccaataatcccgccagcctgaacgaggagaagctgctgctgctgatggcccagctcgaggccctgacccagcgcctgggcgagctgacccagcaggtggctcagctgcaggcggagacgcgggccgcggttgccacggtgaaaaccaaataaaaaatgaatcaataaataaacggagacggttgttgattttaacacagagtcttgaatctttatttgatttttcgcgcgcggtaggccctggaccaccggtctcgatcattgagcacccggtggattttttccaggacccggtagaggtgggcttggatgttgaggtacatgggcatgagcccgtcccgggggtggaggtagctccattgcagggcctcgtgctcgggggtggtgttgtaaatcacccagtcatagcaggggcgcagggcgtggtgctgcacgatgtccttgaggaggagactgatggccacgggcagccccttggtgtaggtgttgacgaacctgttgagctgggagggatgcatgcggggggagatgagatgcatcttggcctggatcttgagattggcgatgttcccgcccagatcccgccgggggttcatgttgtgcaggaccaccagcacggtgtatccggcgcacttggggaatttgtcatgcaacttggaagggaaggcgtgaaagaatttggagacgcccttgtgaccgcccaggttttccatgcactcatccatgatgatggcgatgggcccgtgggcggcggcctgggcaaagacgtttcgggggtcggacacatcgtagttgtggtcctgggtgagctcgtcataggccattttaatgaatttggggcggagggtgcccgactgggggacgaaggtgccctcgatcccgggggcgtagttgccctcgcagatctgcatctcccaggccttgagctcggagggggggatcatgtccacctgcggggcgatgaaaaaaacggtttccggggcgggggagatgagctgggccgaaagcaggttccggagcagctgggacttgccgcagccggtggggccgtagatgaccccgatgaccggctgcaggtggtagttgagggagagacagctgccgtcctcgcggaggaggggggccacctcgttcatcatctcgcgcacatgcatgttctcgcgcacgagttccgccaggaggcgctcgccccccagcgagaggagctcttgcagcgaggcgaagtttttcagcggcttgagyccgtcggccatgggcattttggagagggtctgttgcaagagttccagacggtcccagagctcggtgatgtgctctagggcatctcgatccagcagacctcctcgtttcgcgggttggggcgactgcgggagtagggcaccaggcgatgggcgtccagcgaggccagggtccggtccttccagggtcgcagggtccgcgtcagcgtggtctccgtcacggtgaaggggtgcgcgccgggctgggcgcttgcgagggtgcgcttcaggctcatccggctggtcgagaaccgctcccggtcggcgccctgcgcgtcggccaggtagcaattgagcatgagttcgtagttgagcgcctcggccgcgtggcccttggcgcggagcttacctttggaagtgtgtccgcagacgggacagaggagggacttgagggcgtagagcttgggggcgaggaagacggactcgggggcgtaggcgtccgcgccgcagctggcgcagacggtctcgcactccacgagccaggtgaggtcgggccggttggggtcaaaaacgaggtttcctccgtgctttttgatgcgtttcttacctctggtctccatgagctcgtgtccccgctgggtgacaaagaggctgtccgtgtccccgtagaccgactttatgggccggtcctcgagcggggtgccgcggtcctcgtcgtagaggaaccccgcccactccgagacgaaggcccgggtccaggccagcacgaaggaggccacgtgggaggggtagcggtcgttgtccaccagcgggtccaccttctccagggtatgcaagcacatgtccccctcgtccacatccaggaaggtgattggcttgtaagtgtaggccacgtgaccgggggtcccggccgggggggtataaaagggggcgggcccctgctcgtcctcactgtcttccggatcgctgtccaggagcgccagctgttggggtaggtattccctctcgaaggctggcataacctcggcactcaggttgtcagtttctagaaacgaggaggatttgatattgacggtgccgttggagacgcctttcatgagcccctcgtccatctggtcagaaaagacgatctttttgttgtcgagcttggtggcgaaggagccgtagagggcgttggagaggagcttggcgatggagcgcatggtctggttcttttccttgtcggcgcgctccttggcggcgatgttgagctgcacgtactcgcgcgccacgcacttccattcggggaagacggtggtgagctcgtcgggcacgattctgacccgccagccgcggttgtgcagggtgatgaggtccacgctggtggccacctcgccgcgcaggggctcgttggtccagcagaggcgcccgcccttgcgcgagcagaaggggggcagcgggtccagcatgagctcgtcgggggggtcggcgtccacggtgaagatgccgggcagaagctcggggtcgaagtagctgatgcaggtgtccagatcgtccagcgccgcttgccagtcgcgcacggccagcgcgcgctcgtaggggctgaggggcgtgccccagggcatggggtgcgtgagcgcggaggcgtacatgccgcagatgtcgtagacgtagaggggctcctcgaggacgccgatgtaggtggggtagcagcgccccccgcggatgctggcgcgcacgtagtcgtacagctcgtgcgagggcgcgaggagccccgtgccgaggttggagcgttgcggcttttcggcgcggtagacgatctggcggaagatggcgtgggagttggaggagatggtgggcctctggaagatgttgaagtgggcgtggggcaggccgaccgagtccctgatgaagtgggcgtaggagtcctgcagcttggcgacgagctcggcggtgacgaggacgtccagggcgcagtagtcgagggtctcttggatgatgtcgtacttgagctggcccttctgcttccacagctcgcggttgagaaggaactcttcgcggtccttccagtactcttcgagggggaacccgtcctgatcggcacggtaagagcccaccatgtagaactggttgacggccttgtaggcgcagcagcccttctccacggggagggcgtaagcttgtgcggccttgcgcagggaggtgtgggtgagggcgaaggtgtcgcgcaccatgaccttgaggaactggtgcttgaagtcgaggtcgtcgcagccgccctgctcccagagctggaagtccgtgcgcttcttgtaggcggggttgggcaaagcgaaagtaacatcgttgaagaggatcttgcccgcgcggggcatgaagttgcgagtgatgcggaaaggctggggcacctcggcccggttgttgatgacctgggcggcgaggacgatctcgtcgaagccgttgatgttgtgcccgacgatgtagagttccacgaatcgcgggcggcccttaacgtggggcagcttcttgagctcgtcgtaggtgagctcggcggggtcgctgagcccgtgctgctcgagggcccagtcggcgacgtgggggttggcgctgaggaaggaagtccagagatccacggccagggcggtctgcaagcggtcccggtactgacggaactgctggcccacggccattttttcgggggtgacgcagtagaaggtgcgggggtcgccgtgccagcggtcccacttgagctggagggcgaggtcgtgggcgagctcgacgagcggcgggtccccggagagtttcatgaccagcatgaaggggacgagctgcttgccgaaggaccccatccaggtgtaggtttccacatcgtaggtgaggaagagcctttcggtgcgaggatgcgagccgatggggaagaactggatctcctgccaccagttggaggaatggctgttgatgtgatggaagtagaaatgccgacggcgcgccgagcactcgtgcttgtgtttatacaagcgtccgcagtgctcgcaacgctgcacgggatgcacgtgctgcacgagctgtacctgggttcctttgacgaggaatttcagtgggcagtggagcgctggcggctgcatctggtgctgtactacgtcctggccatcggcgtggccatcgtctgcctcgatggtggtcatgctgacgagcccgcgcgggaggcaggtccagacttcggctcggacgggtcggagagcgaggacgagggcgcgcaggccggagctgtccagggtcctgagacgctgcggagtcaggtcagtgggcagcggcggcgcgcggttgacttgcaggagcttttccagggcgcgcgggaggtccagatggtacttgatctccacggcgccgttggtggcgacgtccacggcttgcagggtcccgtgcccctggggcgccaccaccgtgccccgtttcttcttgggcgctgcttccatgccggtcagaagcggcggcgaggacgcgcgccgggcggcaggggcggctcgggacccggaggcaggggcggcaggggcacgtcggcgccgcgcgcgggcaggttctggtactgcgcccggagaagactggcgtgagcgacgacgcgacggttgacgtcctggatctgacgcctctgggtgaaggccacgggacccgtgagtttgaacctgaaagagagttcgacagaatcaatctcggtatcgttgacggcggcctgccgcaggatctcttgcacgtcgcccgagttgtcctggtaggcgatctcggtcatgaactgctcgatctcctcctcctgaaggtctccgcggccggcgcgctcgacggtggccgcgaggtcgttggagatgcggcccatgagctgcgagaaggcgttcatgccggcctcgttccagacgcggctgtagaccacggctccgtcggggtcgcgcgcgcgcatgaccacctgggcgaggttgagctcgacgtggcgcgtgaagaccgcgtagttgcagaggcgctggtagaggtagttgagcgtggtggcgatgtgctcggtgacgaagaagtacatgatccagcggcggagcggcatctcgctgacgtcgcccagggcttccaagcgctccatggcctcgtagaagtccacggcgaagttgaaaaactgggagttgcgcgccgagacggtcaactcctcctccagaagacggatgagctcagcgatggtggcgcgcacctcgcgctcgaaggccccggggggctcctcttcttccatctcttcctcctccactaacatctcttctacttcctcctcaggaggcggcggcgggggaggggccctgcgtcgccggcggcgcacgggcagacggtcgatgaagcgctcgatggtctccccgcgccggcgacgcatggtctcggtgacggcgcgcccgtcctcgcggggccgcagcgtgaagacgccgccgcgcatctccaggtggccgccgggggggtctccgttgggcagggagagggcgctgacgatgcatcttatcaattggcccgtagggactccgcgcaaggacctgagcgtctcgagatccacgggatccgaaaaccgctgaacgaaggcttcgagccagtcgcagtcgcaaggtaggctgagcccggtttcttgttcttcggggatttcgggaggcgggcgggcgatgctgctggtgatgaagttgaagtaggcggtcctgagacggcggatggtggcgaggagcaccaggtccttgggcccggcttgctggatgcgcagacggtcggccatgccccaggcgtggtcctgacacctggcgaggtccttgtagtagtcctgcatgagccgctccacgggcacctcctcctcgcccgcgcggccgtgcatgcgcgtgagcccgaacccgcgctggggctggacgagcgccaggtcggcgacgacgcgctcggcgaggatggcctgctgtatctgggtgagggtggtctggaagtcgtcgaagtcgacgaagcggtggtaggctccggtgttgatggtataggagcagttggccatgacggaccagttgacggtctggtggccgggtcgcacgagctcgtggtacttgaggcgcgagtaggcgcgcgtgtcgaagatgtagtcgttgcaggtgcgcacgaggtactggtatccgacgaggaagtgcggcggcggctggcggtagagcggccatcgctcggtggcgggggcgccgggcgcgaggtcctcgagcatgaggcggtggtagccgtagatgtacctggacatccaggtgatgccggcggcggtggtggaggcgcgcgggaactcgcggacgcggttccagatgttgcgcagcggcaggaagtagttcatggtggccgcggtctggcccgtgaggcgcgcgcagtcgtggatgctctagacatacgggcaaaaacgaaagcggtcagcggctcgactccgtggcctggaggctaagcgaacgggttgggctgcgcgtgtaccccggttcgaatctcgaatcaggctggagccgcagctaacgtggtactggcactcccgtctcgacccaagcctgctaacgaaacctccaggatacggaggcgggtcgttttttggccttggtcgctggtcatgaaaaactagtaagcgcggaaagcgaccgcccgcgatggctcgctgccgtagtctggagaaagaatcgccagggttgcgttgcggtgtgccccggttcgagcctcagcgctcggcgccggccggattccgcggctaacgtgggcgtggctgccccgtcgtttccaagaccccttagccagccgacttctccagttacggagcgagcccctctttttcttgtgtttttgccagatgcatcccgtactgcggcagatgcgcccccaccctccacctcaaccgcccctaccgccgcagcagcagcaacagccggcgcttctgcccccgccccagcagcagccagccactaccgcggcggccgccgtgagcggagccggcgttcagtatgacctggccttggaagagggcgaggggctggcgcggctgggggcgtcgtcgccggagcggcacccgcgcgtgcagatgaaaagggacgctcgcgaggcctacgtgcccaagcagaacctgttcagagacaggagcggcgaggagcccgaggagatgcgcgcctcccgcttccacgcggggcgggagctgcggcgcggcctggaccgaaagcgggtgctgagggacgaggatttcgaggcggacgagctgacggggatcagccccgcgcgcgcgcacgtggccgcggccaacctggtcacggcgtacgagcagaccgtgaaggaggagagcaacttccaaaaatccttcaacaaccacgtgcgcacgctgatcgcgcgcgaggaggtgaccctgggcctgatgcacctgtgggacctgctggaggccatcgtgcagaaccccacgagcaagccgctgacggcgcagctgtttctggtggtgcagcacagtcgggacaacgagacgttcagggaggcgctgctgaatatcaccgagcccgagggccgctggctcctggacctggtgaacattctgcagagcatcgtggtgcaggagcgcgggctgccgctgtccgagaagctggcggctatcaacttctcggtgctgagcctgggcaagtactacgctaggaagatctacaagaccccgtacgtgcccatagacaaggaggtgaagatcgacgggttttacatgcgcatgaccctgaaagtgctgaccctgagcgacgatctgggggtgtaccgcaacgacaggatgcaccgcgcggtgagcgccagccgccggcgcgagctgagcgaccaggagctgatgcacagcctgcagcgggccctgaccggggccgggaccgagggggagagctactttgacatgggcgcggacctgcgctggcagcccagccgccgggccttggaagctgccggcggttccccctacgtggaggaggtggacgatgaggaggaggagggcgagtacctggaagactgatggcgcgaccgtatttttgctagatgcagcaacagccaccgcctcctgatcccgcgatgcgggcggcgctgcagagccagccgtccggcattaactcctcggacgattggacccaggccatgcaacgcatcatggcgctgacgacccgcaatcccgaagcctttagacagcagcctcaggccaaccggctctcggccatcctggaggccgtggtgccctcgcgctcgaaccccacgcacgagaaggtgctggccatcgtgaacgcgctggtggagaacaaggccatccgcggcgacgaggccgggctggtgtacaacgcgctgctggagcgcgtggcccgctacaacagcaccaacgtgcagacgaacctggaccgcatggtgaccgacgtgcgcgaggcggtgtcgcagcgcgagcggttccaccgcgagtcgaacctgggctccatggtggcgctgaacgccttcctgagcacgcagcccgccaacgtgccccggggccaggaggactacaccaacttcatcagcgcgctgcggctgatggtggccgaggtgccccagagcgaggtgtaccagtcggggccggactacttcttccagaccagtcgccagggcttgcagaccgtgaacctgagccaggctttcaagaacttgcagggactgtggggcgtgcaggccccggtcggggaccgcgcgacggtgtcgagcctgctgacgccgaactcgcgcctgctgctgctgctggtggcgcccttcacggacagcggcagcgtgagccgcgactcgtacctgggctacctgcttaacctgtaccgcgaggccatcgggcaggcgcacgtggacgagcagacctaccaggagatcacccacgtgagccgcgcgctgggccaggaggacccgggcaacctggaggccaccctgaacttcctgctgaccaaccggtcgcagaagatcccgccccagtacgcgctgagcaccgaggaggagcgcatcctgcgctacgtgcagcagagcgtggggctgttcctgatgcaggagggggccacgcccagcgccgcgctcgacatgaccgcgcgcaacatggagcccagcatgtacgctcgcaaccgcccgttcatcaataagctgatggactacttgcatcgggcggccgccatgaactcggactactttaccaacgccatcttgaacccgcactggctcccgccgcccgggttctacacgggcgagtacgacatgcccgaccccaacgacgggttcctgtgggacgacgtggacagcagcgtgttctcgccgcgccccgccaccaccgtgtggaagaaagagggcggggaccggcggccgtcctcggcgctgtccggtcgcgcgggtgctgccgcggcggtgcctgaggccgccagccccttcccgagcctgcccttttcgctgaacagcgtgcgcagcagcgagctgggtcggctgacgcggccgcgcctgctgggcgaggaggagtacctgaacgactccttgttgaggcccgagcgcgagaagaacttccccaataacgggatagagagcctggtggacaagatgagccgctggaagacgtacgcgcacgagcacagggacgagccccgagctagcagcagcgcaggcacccgtagacgccagcgacacgacaggcagcggggtctggtgtgggacgatgaggattccgccgacgacagcagcgtgttggacttgggtgggagtggtggtggtaacccgttcgctcacttgcgcccccgtatcgggcgcctgatgtaagaatctgaaaaaataaaaaacggtactcaccaaggccatggcgaccagcgtgcgttcttctctgttgtttgtagtagtatgatgaggcgcgtgtacccggagggtcctcctccctcgtacgagagcgtgatgcagcaggcggtggcggcggcgatgcagcccccgctggaggcgccttacgtgcccccgcggtacctggcgcctacggaggggcggaacagcattcgttactcggagctggcacccttgtacgataccacccggttgtacctggtggacaacaagtcggcggacatcgcctcgctgaactaccagaacgaccacagcaacttcctgaccaccgtggtgcagaacaacgatttcacccccacggaggccagcacccagaccatcaactttgacgagcgctcgcggtggggcggccagctgaaaaccatcatgcacaccaacatgcccaacgtgaacgagttcatgtacagcaacaagttcaaggcgcgggtgatggtctcgcgcaagacccccaatggggtcgcggtggatgagaattatgatggtagtcaggacgagctgacttacgagtgggtggagtttgagctgcccgagggcaacttctcggtgaccatgaccatcgatctgatgaacaacgccatcatcgacaactacttggcggtggggcgtcagaacggggtgctggagagcgacatcggcgtgaagttcgacacgcgcaacttccggctgggctgggaccccgtgaccgagctggtgatgccgggcgtgtacaccaacgaggccttccaccccgacatcgtcctgctgcccggctgcggcgtggacttcaccgagagccgcctcagcaacctgctgggcatccgcaagcggcagcccttccaggagggcttccagatcctgtacgaggacctggaggggggcaacatccccgcgctcttggatgtcgaagcctatgagaaaagcaaggaggaggccgccgcagcggcgaccgcagccgtggccaccgcctctaccgaggtgcggggcgataattttgctagcgccgcggcagtggccgaggcggctgaaaccgaaagtaagatagtcatccagccggtggagaaggacagcaaggacaggagctacaacgtgctcgcggacaagaaaaacaccgcctaccgcagctggtacctggcctacaactacggcgaccccgagaagggcgtgcgctcctggacgctgctcaccacctcggacgtcacctgcggcgtggagcaagtctactggtcgctgcccgacatgatgcaagacccggtcaccttccgctccacgcgtcaagttagcaactacccggtggtgggcgccgagctcctgcccgtctactccaagagcttcttcaacgagcaggccgtctactcgcagcagctgcgcgccttcacctcgctcacgcacgtcttcaaccgcttccccgagaaccagatcctcgtccgcccgcccgcgcccaccattaccaccgtcagtgaaaacgttcctgctctcacagatcacgggaccctgccgctgcgcagcagtatccggggagtccagcgcgtgaccgtcactgacgccagacgccgcacctgcccctacgtctacaaggccctgggcgtagtcgcgccgcgcgtcctctcgagccgcaccttctaaaaaatgtccattctcatctcgcccagtaataacaccggttggggcctgcgcgcgcccagcaagatgtacggaggcgctcgccaacgctccacgcaacaccccgtgcgcgtgcgcgggcacttccgcgctccctggggcgccctcaagggccgcgtgcgctcgcgcaccaccgtcgacgacgtgatcgaccaggtggtggccgacgcgcgcaactacacgcccgccgccgcgcccgcctccaccgtggacgccgtcatcgacagcgtggtggccgatgcgcgccggtacgcccgcgccaagagccggcggcggcgcatcgcccggcggcaccggagcacccccgccatgcgcgcggcgcgagccttgctgcgcagggccaggcgcacgggacgcagggccatgctcagggcggccagacgcgcggcctccggcagcagcagcgccggcaggacccgcagacgcgcggccacggcggcggcggcggccatcgccagcatgtcccgcccgcggcgcggcaacgtgtactgggtgcgcgacgccgccaccggtgtgcgcgtgcccgtgcgcacccgcccccctcgcacttgaagatgctgacttcgcgatgttgatgtgtcccagcggcgaggaggatgtccaagcgcaaatacaaggaagagatgctccaggtcatcgcgcctgagatctacggccccgcggtgaaggaggaaagaaagccccgcaaactgaagcgggtcaaaaaggacaaaaaggaggaggaagatgtggacggactggtggagtttgtgcgcgagttcgccccccggcggcgcgtgcagtggcgcgggcggaaagtgaaaccggtgctgcggcccggcaccacggtggtcttcacgcccggcgagcgttccggctccgcctccaagcgctcctacgacgaggtgtacggggacgaggacatcctcgagcaggcggtcgagcgtctgggcgagtttgcttacggcaagcgcagccgccccgcgcccttgaaagaggaggcggtgtccatcccgctggaccacggcaaccccacgccgagcctgaagccggtgaccctgcagcaggtgctgccgagcgcggcgccgcgccggggcttcaagcgcgagggcggcgaggatctgtacccgaccatgcagctgatggtgcccaagcgccagaagctggaggacgtgctggagcacatgaaggtggaccccgaggtgcagcccgaggtcaaggtgcggcccatcaagcaggtggccccgggcctgggcgtgcagaccgtggacatcaagatccccacggagcccatggaaacgcagaccgagcccgtgaagcccagcaccagcaccatggaggtgcagacggatccctggatgccggcgccggcttccaccactcgccgaagacgcaagtacggcgcggccagcctgctgatgcccaactacgcgctgcatccttccatcatccccacgccgggctaccgcggcacgcgcttctaccgcggctacaccagcagccgccgcaagaccaccacccgccgccgccgtcgtcgcacccgccgcagcagcaccgcgacttccgccgccgccctggtgcggagagtgtaccgcagcgggcgcgagcctctgaccctgccgcgcgcgcgctaccacccgagcatcgccatttaactctgccgtcgcctcctacttgcagatatggccctcacatgccgcctccgcgtccccattacgggctaccgaggaagaaagccgcgccgtagaaggctgacggggaacgggctgcgtcgccatcaccaccggcggcggcgcgccatcagcaagcggttggggggaggcttcctgcccgcgctgatccccatcatcgccgcggcgatcggggcgatccccggcatagcttccgtggcggtgcaggcctctcagcgccactgagacacagcttggaaaatttgtaataaaaaaatggactgacgctcctggtcctgtgatgtgtgtttttagatggaagacatcaatttttcgtccctggcaccgcgacacggcacgcggccgtttatgggcacctggagcgacatcggcaacagccaactgaacgggggcgccttcaattggagcagtctctggagcgggcttaagaatttcgggtccacgctcaaaacctatggcaacaaggcgtggaacagcagcacagggcaggcgctgagggaaaagctgaaagagcagaacttccagcagaaggtggtcgatggcctggcctcgggcatcaacggggtggtggacctggccaaccaggccgtgcagaaacagatcaacagccgcctggacgcggtcccgcccgcggggtccgtggagatgccccaggtggaggaggagctgcctcccctggacaagcgcggcgacaagcgaccgcgtcccgacgcggaggagacgctgctgacgcacacggacgagccgcccccgtacgaggaggcggtgaaactgggtctgcccaccacgcggcccgtggcgcctctggccaccggggtgctgaaacccagcagcagcagccagcccgcgaccctggacttgcctccgcctgcttcccgcccctccacagtggctaagcccctgccgccggtggccgtcgcgtcgcgcgccccccgaggccgcccccaggcgaactggcagagcactctgaacagcatcgtgggtctgggagtgcagagtgtgaagcgccgccgctgctattaaaagacactgtagcgcttaacttgcttgtctgtgtgtatatgtatgtccgccgaccagaaggaggaagaggcgcgtcgccgagttgcaagatggccaccccatcgatgctgccccagtgggcgtacatgcacatcgccggacaggacgcttcggagtacctgagtccgggtctggtgcagttcgcccgcgccacagacacctacttcagtctggggaacaagtttaggaaccccacggtggcgcccacgcacgatgtgaccaccgaccgcagccagcggctgacgctgcgcttcgtgcccgtggaccgcgaggacaacacctactcgtacaaagtgcgctacacgctggccgtgggcgacaaccgcgtgctggacatggccagcacctactttgacatccgcggcgtgctggatcgggggcccagcttcaaaccctactccggcaccgcctacaacagcctggctcccaagggagcgcccaacacttgccagtggacatataaagctggtgatactgatacagaaaaaacctatacatatggaaatgcacctgtgcaaggcattagcattacaaaggatggtattcaacttggaactgacagcgatggtcaggcaatctatgcagacgaaacttatcaaccagagcctcaagtgggtgatgctgaatggcatgacatcactggtactgatgaaaaatatggaggcagagctcttaagcctgacaccaaaatgaagccttgctatggttcttttgccaagcctaccaataaagaaggaggccaggcaaatgtgaaaaccgaaacaggcggtaccaaagaatatgacattgacatggcattcttcgataatcgaagtgcagctgccgccggcctagccccagaaattgttttgtatactgagaatgtggatctggaaactccagatacccatattgtatacaaggcaggtacagatgacagtagctcttctatcaatttgggtcagcagtccatgcccaacagacccaactacattggcttcagagacaactttatcggtctgatgtactacaacagcactggcaatatgggtgtactggctggacaggcctcccagctgaatgctgtggtggacttgcaggacagaaacaccgaactgtcctaccagctcttgcttgactctctgggtgacagaaccaggtatttcagtatgtggaatcaggcggtggacagttatgaccccgatgtgcgcattattgaaaatcacggtgtggaggatgaacttcctaactattgcttccccctggatgctgtgggtagaactgatacttaccagggaattaaggccaatggtgataatcaaaccacctggaccaaagatgatactgttaatgatgctaatgaattgggcaagggcaatcctttcgccatggagatcaacatccaggccaacctgtggcggaacttcctctacgcgaacgtggcgctgtacctgcccgactcctacaagtacacgccggccaacatcacgctgcccaccaacaccaacacctacgattacatgaacggccgcgtggtggcgccctcgctggtggacgcctacatcaacatcggggcgcgctggtcgctggaccccatggacaacgtcaaccccttcaaccaccaccgcaacgcgggcctgcgataccgctccatgctcctgggcaacgggcgctacgtgcccttccacatccaggtgccccaaaagtttttcgccatcaagagcctcctgctcctgcccgggtcctacacctacgagtggaacttccgcaaggacgtcaacatgatcctgcagagctccctcggcaacgacctgcgcacggacggggcctccatcgccttcaccagcatcaacctctacgccaccttcttccccatggcgcacaacaccgcctccacgctcgaggccatgctgcgcaacgacaccaacgaccagtccttcaacgactacctctcggcggccaacatgctctaccccatcccggccaacgccaccaacgtgcccatctccatcccctcgcgcaactgggccgccttccgcggctggtccttcacgcgcctcaagacccgcgagacgccctcgctcggctccgggttcgacccctacttcgtctactcgggctccatcccctacctcgacggcaccttctacctcaaccacaccttcaagaaggtctccatcaccttcgactcctccgtcagctggcccggcaacgaccgcctcctgacgcccaacgagttcgaaatcaagcgcaccgtcgacggagaggggtacaacgtggcccagtgcaacatgaccaaggactggttcctggtccagatgctggcccactacaacatcggctaccagggcttctacgtgcccgagggctacaaggaccgcatgtactccttcttccgcaacttccagcccatgagccgccaggtcgtggacgaggtcaactacaaggactaccaggccgtcaccctggcctaccagcacaacaactcgggcttcgtcggctacctcgcgcccaccatgcgccagggccagccctaccccgccaactacccctacccgctcatcggcaagagcgccgtcgccagcgtcacccagaaaaagttcctctgcgaccgggtcatgtggcgcatccccttctccagcaacttcatgtccatgggcgcgctcaccgacctcggccagaacatgctctacgccaactccgcccacgcgctagacatgaatttcgaagtcgaccccatggatgagtccacccttctctatgttgtcttcgaagtcttcgacgtcgtccgagtgcaccagccccaccgcggcgtcatcgaggccgtctacctgcgcacgcccttctcggccggcaacgccaccacctaagcctcttgcttcttgcaagatgacggcctgcgcgggctccggcgagcaggagctcagggccatcctccgcgacctgggctgcgggccctgcttcctgggcaccttcgacaagcgcttcccgggattcatggccccgcacaagctggcctgcgccatcgtcaacacggccggccgcgagaccgggggcgagcactggctggccttcgcctggaacccgcgctcccacacctgctacctcttcgaccccttcgggttctcggacgagcgcctcaagcagatctaccagttcgagtacgagggcctgctgcgtcgcagcgccctggccaccgaggaccgctgcgtcaccctggaaaagtccacccagaccgtgcagggtccgcgctcggccgcctgcgggctcttctgctgcatgttcctgcacgccttcgtgcactggcccgaccgccccatggacaagaaccccaccatgaacttgctgacgggggtgcccaacggcatgctccagtcgccccaggtggaacccaccctgcgccgcaaccaggaggcgctctaccgcttcctcaacgcccactccgcctactttcgctcccaccgcgcgcgcatcgagaaggccaccgccttcgaccgcatgaatcaagacatgtaatccggtgtgtgtatgtgaatgctttattcatcataataaacagcacatgtttatgccaccttctctgaggctctgactttatttagaaatcgaaggggttctgccggctctcggcatggcccgcgggcagggatacgttgcggaactggtacttgggcagccacttgaactcggggatcagcagcttcggcacggggaggtcggggaacgagtcgctccacagcttgcgcgtgagttgcagggcgcccagcaggtcgggcgcggagatcttgaaatcgcagttgggacccgcgttctgcgcgcgagagttacggtacacggggttgcagcactggaacaccatcagggccgggtgcttcacgctcgccagcaccgtcgcgtcggtgatgccctccacgtccagatcctcggcgttggccatcccgaagggggtcatcttgcaggtctgccgccccatgctgggcacgcagccgggcttgtggttgcaatcgcagtgcagggggatcagcatcatctgggcctgctcggagctcatgcccgggtacatggccttcatgaaagcctccagctggcggaaggcctgctgcgccttgccgccctcggtgaagaagaccccgcaggacttgctagagaactggttggtggcgcagccagcgtcgtgcacgcagcagcgcgcgtcgttgttggccagctgcaccacgctgcgcccccagcggttctgggtgatcttggcccggtcggggttctccttcagcgcgcgctgcccgttctcgctcgccacatccatctcgatcgtgtgctccttctggatcatcacggtcccgtgcaggcaccgcagcttgccctcggcctcggtgcacccgtgcagccacagcgcgcagccggtgctctcccagttcttgtgggcgatctgggagtgcgagtgcacgaagccctgcaggaagcggcccatcatcgtggtcagggtcttgttgctggtgaaggtcagcggaatgccgcggtgctcctcgttcacatacaggtggcagatacggcggtacacctcgccctgctcgggcatcagctggaaggcggacttcaggtcgctctccacgcggtaccggtccatcagcagcgtcatcacttccatgcccttctcccaggccgaaacgatcggcaggctcagggggttcttcaccgttgtcatcttagtcgccgccgccgaagtcagggggtcgttctcgtccagggtctcaaacactcgcttgccgtccttctcggtgatgcgcacggggggaaagctgaagcccacggccgccagctcctcctcggcctgcctttcgtcctcgctgtcctggctgatgtcttgcaaaggcacatgcttggtcttgcggggtttctttttgggcggcagaggcggcggcggagacgtgctgggcgagcgcgagttctcgctcaccacgactatttcttctccttggccgtcgtccgagaccacgcggcggtaggcatgcctcttctggggcagaggcggaggcgacgggctctcgcggttcggcgggcggctggcagagccccttccgcgttcgggggtgcgctcctggcggcgctgctctgactgacttcctccgcggccggccattgtgttctcctagggagcaagcatggagactcagccatcgtcgccaacatcgccatctgcccccgccgccgccgacgagaaccagcagcagcagaatgaaagcttaaccgccccgccgcccagccccacctccgacgccgcagccccagacatgcaagagatggaggaatccatcgagattgacctgggctacgtgacgcccgcggagcacgaggaggagctggcagcgcgcttttcagccccggaagagaaccaccaagagcagccagagcaggaagcagagagcgagcagaaccaggctgggctcgagcatggcgactacctgagcggggcagaggacgtgctcatcaagcatctggcccgccaatgcatcatcgtcaaggacgcgctgctcgaccgcgccgaggtgcccctcagcgtggcggagctcagccgcgcctacgagcgcaacctcttctcgccgcgcgtgccccccaagcgccagcccaacggcacctgcgagcccaacccgcgcctcaacttctacccggtcttcgcggtgcccgaggccctggccacctaccacctctttttcaagaaccaaaggatccccgtctcctgccgcgccaaccgcacccgcgccgacgccctgctcaacctgggccccggcgcccgcctacctgatatcgcctccttggaagaggttcccaagatcttcgagggtctgggcagcgacgagactcgggccgcgaacgctctgcaaggaagcggagaggagcatgagcaccacagcgccctggtggagttggaaggcgacaacgcgcgcctggcggtcctcaagcgcacggtcgagctgacccacttcgcctacccggcgctcaacctgccccccaaggtcatgagcgccgtcatggaccaggtgctcatcaagcgcgcctcgcccctctcggaggaggagatgcaggaccccgagagctcggacgagggcaagcccgtggtcagcgacgagcagctggcgcgctggctgggagcgagtagcaccccccagagcctggaagagcggcgcaagctcatgatggccgtggtcctggtgaccgtggagctggagtgtctgcgccgcttcttcgccgacgcggagaccctgcgcaaggtcgaggagaacctgcactacctcttcagacacgggttcgtgcgccaggcctgcaagatctccaacgtggagctgaccaacctggtctcctacatgggcatcctgcacgagaaccgcctggggcagaacgtgctgcacaccaccctgcgcggggaggcccgccgcgactacatccgcgactgcgtctacctgtacctctgccacacctggcagacgggcatgggcgtgtggcagcagtgcctggaggagcagaacctgaaagagctctgcaagctcctgcagaagaacctcaaggccctgtggaccgggttcgacgagcgcaccaccgccgcggacctggccgacctcatcttccccgagcgcctgcggctgacgctgcgcaacgggctgcccgactttatgagccaaagcatgttgcaaaactttcgctctttcatcctcgaacgctccgggatcctgcccgccacctgctccgcgctgccctcggacttcgtgccgctgaccttccgcgagtgccccccgccgctctggagccactgctacctgctgcgcctggccaactacctggcctaccactcggacgtgatcgaggacgtcagcggcgagggcctgctcgagtgccactgccgctgcaacctctgcacgccgcaccgctccctggcctgcaacccccagctgctgagcgagacccagatcatcggcaccttcgagttgcaaggccccggcgagggcaaggggggtctgaaactcaccccggggctgtggacctcggcctacttgcgcaagttcgtgcccgaggactaccatcccttcgagatcaggttctacgaggaccaatcccagccgcccaaggccgagctgtcggcctgcgtcatcacccagggggccatcctggcccaattgcaagccatccagaaatcccgccaagaatttctgctgaaaaagggccacggggtctacttggacccccagaccggagaggagctcaaccccagcttcccccaggatgccccgaggaagcagcaagaagctgaaagtggagctgccgccgccgccggaggatttggaggaagactgggagagcagtcaggcagaggaggaggagatggaagactgggacagcactcaggcagaggaggacagcctgcaagacagtctggaggaggaagacgaggtggaggaggcagaggaagaagcagccgccgccagaccgtcgtcctcggcggaggaggagaaagcaagcagcacggataccatctccgctccgggtcggggtcgcggcggccgggcccacagtagatgggacgagaccgggcgcttcccgaaccccaccacccagaccggtaagaaggagcggcagggatacaagtcctggcgggggcacaaaaacgccatcgtctcctgcttgcaagcctgcgggggcaacatctccttcacccggcgctacctgctcttccaccgcggggtgaacttcccccgcaacatcttgcattactaccgtcacctccacagcccctactactgtttccaagaagaggcagaaacccagcagcagcagcagcagcagaaaaccagcggcagcagctagaaaatccacagcggcggcaggtggactgaggatcgcggcgaacgagccggcgcagacccgggagctgaggaaccggatctttcccaccctctatgccatcttccagcagagtcgggggcaagagcaggaactgaaagtcaagaaccgttctctgcgctcgctcacccgcagttgtctgtatcacaagagcgaagaccaacttcagcgcactctcgaggacgccgaggctctcttcaacaagtactgcgcgctcactcttaaagagtagcccgcgcccgcccacacacggaaaaaggcgggaattacgtcaccacctgcgcccttcgcccgaccatcatcatgagcaaagagattcccacgccttacatgtggagctaccagccccagatgggcctggccgccggcgccgcccaggactactccacccgcatgaactggctcagtgccgggcccgcgatgatctcacgggtgaatgacatccgcgcccaccgaaaccagatactcctagaacagtcagcgatcaccgccacgccccgccatcaccttaatccgcgtaattggcccgccgccctggtgtaccaggaaattccccagcccacgaccgtactacttccgcgagacgcccaggccgaagtccagctgactaactcaggtgtccagctggccggcggcgccgccctgtgtcgtcaccgccccgctcagggtataaagcggctggtgatccgaggcagaggcacacagctcaacgacgaggtggtgagctcttcgctgggtctgcgacctgacggagtcttccaactcgccggatcggggagatcttccttcacgcctcgtcaggccgtcctgactttggagagttcgtcctcgcagccccgctcgggtggcatcggcactctccagttcgtggaggagttcactccctcggtctacttcaaccccttctccggctcccccggccactacccggacgagttcatcccgaacttcgacgccatcagcgagtcggtggacggctacgattgaatgtcccatggtggcgcggctgacctagctcggcttcgacacctggaccactgccgccgcttccgctgcttcgctcgggatctcgccgagtttgcctactttgagctgcccgaggagcaccctcagggcccggcccacggagtgcggatcgtcgtcgaagggggtctcgactcccacctgcttcggatcttcagccagcgtccgatcctggccgagcgcgagcaaggacagacccttctgaccctgtactgcatctgcaaccaccccggcctgcatgaaagtctttgttgtctgctgtgtactgagtataataaaagctgagatcagcgactactccggacttccgtgtgttcctgctatcaaccagtccctgttcttcaccgggaacgagaccgagctccagctccagtgtaagccccacaagaagtacctcacctggctgttccagggctctccgatcgccgttgtcaaccactgcgacaacgacggagtcctgctgagcggccctgccaaccttactttttccacccgcagaagcaagctccagctcttccaacccttcctccccgggacctatcagtgcgtctcgggaccctgccatcacaccttccacctgatcccgaataccacagcgtcgctccccgctactaacaaccaaactacccaccaacgccaccgtcgcgaccgcggacatgtacagagctcgagaagtactaggccacaatacatgcccatattagactatgaggccgagccacagcgacccatgctccccgctattagttacttcaatctaaccggcggagatgactgacccactggccaacaacaacgtcaacgaccttctcctggacatggacggccgcgcctcggagcagcgactcgcccaacttcgcattcgccagcagcaggagagagccgtcaaggagctgcaggacggcatagccatccaccagtgcaagaaaggcatcttctgcctggtgaaacaggccaagatctcctacgaggtcaccccgaccgaccatcgcctctcctacgagctcctgcagcagcgccagaagttcacctgcctggtcggagtcaaccccatcgtcatcacccagcagtcgggcgataccaaggggtgcatccactgctcctgcgactcccccgactgcgtccacactctgatcaagaccctctgcggcctccgcgacctcctccccatgaactaatcacccccttatccagtgaaataaatatcatattgatgatgatttaaataaaaaataatcatttgatttgaaataaagatacaatcatattgatgatttgagttttaaaaaataaagaatcacttacttgaaatctgataccaggtctctgtccatgttttctgccaacaccacctcactcccctcttcccagctctggtactgcagaccccggcgggctgcaaacttcctccacacgctgaaggggatgtcaaattcctcctgtccctcaatcttcattttatcttctatcagatgtccaaaaagcgcgtccgggtggatgatgacttcgaccccgtctacccctacgatgcagacaacgcaccgaccgtgcccttcatcaacccccccttcgtctcttcagatggattccaagagaagcccctgggggtgctgtccctgcgactggctgaccccgtcaccaccaagaacggggaaatcaccctcaagctgggagagggggtggacctcgactcctcgggaaaactcatctccaacacggccaccaaggccgccgcccctctcagtttttccaacaacaccatttcccttaacatggatacccctctttataccaaagatggaaaattatccttacaagtttctccaccgttaaacatattaaaatcaaccattctgaacacattagctgtagcttatggatcaggtttaggactgagtggtggcactgctcttgcagtacagttggcctctccactcacttttgatgaaaaaggaaatattaaaattaacctagccagtggtccattaacagttgatgcaagtcgacttagtatcaactgcaaaagaggggtcactgtcactacctcaggagatgcaattgaaagcaacataagctggcctaaaggtataagatttgaaggtaatggcatagctgcaaacattggcagaggattggaatttggaaccactagtacagagactgatgtcacagatgcatacccaattcaagttaaattgggtactggccttacctttgacagtacaggcgccattgttgcttggaacaaagaggatgataaacttacattatggaccacagccgacccctcgccaaattgcaaaatatactctgaaaaagatgccaaactcacactttgcttgacaaagtgtggaagtcaaattctgggtactgtgactgtattggcagtgaataatggaagtctcaacccaatcacaaacacagtaagcactgcactcgtctccctcaagtttgatgcaagtggagttttgctaagcagctccacattagacaaagaatattggaacttcagaaagggagatgttacacctgctgagccctatactaatgctataggttttatgcctaacataaaggcctatcctaaaaacacatctgcagcttcaaaaagccatattgtcagtcaagtttatctcaatggggatgaggccaaaccactgatgctgattattacttttaatgaaactgaggatgcaacttgcacctacagtatcacttttcaatggaaatgggatagtactaagtacacaggtgaaacacttgctaccagctccttcaccttctcctacatcgcccaagaatgaacactgtatcccaccctgcatgccaacccttcccaccccactctgtctatggaaaaaactctgaagcacaaaataaaataaagttcaagtgttttattgattcaacagttttacaggattcgagcagttatttttcctccaccctcccaggacatggaatacaccaccctctccccccgcacagccttgaacatctgaatgccattggtgatggacatgcttttggtctccacgttccacacagtttcagagcgagccagtctcgggtcggtcagggagatgaaaccctccgggcactcccgcatctgcacctcacagctcaacagctgaggattgtcctcggtggtcgggatcacggttatctggaagaagcagaagagcggcggtgggaatcatagtccgcgaacgggatcggccggtggtgtcgcatcaggccccgcagcagtcgctgccgccgccgctccgtcaagctgctgctcagggggtccgggtccagggactccctcagcatgatgcccacggccctcagcatcagtcgtctggtgcggcgggcgcagcagcgcatgcggatctcgctcaggtcgctgcagtacgtgcaacacaggaccaccaggttgttcaacagtccatagttcaacacgctccagccgaaactcatcgcgggaaggatgctacccacgtggccgtcgtaccagatcctcaggtaaatcaagtggcgctccctccagaacacgctgcccacgtacatgatctccttgggcatgtggcggttcaccacctcccggtaccacatcaccctctggttgaacatgcagccccggatgatcctgcggaaccacagggccagcaccgccccgcccgccatgcagcgaagagaccccgggtcccggcaatggcaatggaggacccaccgctcgtacccgtggatcatctgggagctgaacaagtctatgttggcacagcacaggcatatgctcatgcatctcttcagcactctcagctcctcgggggtcaaaaccatatcccagggcacggggaactcttgcaggacagcgaaccccgcagaacagggcaatcctcgcacataacttacattgtgcatggacagggtatcgcaatcaggcagcaccgggtgatcctccaccagagaagcgcgggtctcggtctcctcacagcgtggtaagggggccggccgatacgggtgatggcgggacgcggctgatcgtgttcgcgaccgtgtcatgatgcagttgctttcggacattttcgtacttgctgtagcagaacctggtccgggcgctgcacaccgatcgccggcggcggtcccggcgcttggaacgctcggtgttgaaattgtaaaacagccactctctcagaccgtgcagcagatctagggcctcaggagtgatgaagatcccatcatgcctgatagctctgatcacatcgaccaccgtggaatgggccagacccagccagatgatgcaattttgttgggtttcggtgacggcgggggagggaagaacaggaagaaccatgattaacttttaatccaaacggtctcggagcacttcaaaatgaaggtcgcggagatggcacctctcgcccccgctgtgttggtggaaaataacagccaggtcaaaggtgatacggttctcgagatgttccacggtggcttccagcaaagcctccacgcgcacatccagaaacaagacaatagcgaaagcgggagggttctctaattcctcaatcatcatgttacactcctgcaccatccccagataattttcatttttccagccttgaatgattcgaactagttcctgaggtaaatccaagccagccatgataaagagctcgcgcagagcgccctccaccggcattcttaagcacaccctcataattccaagatattctgctcctggttcacctgcagcagattgacaagcggaatatcaaaatctctgccgcgatccctaagctcctccctcagcaataactgtaagtactctttcatatcctctccgaaatttttagccataggaccaccaggaataagattagggcaagccacagtacagataaaccgaagtcctccccagtgagcattgccaaatgcaagactgctataagcatgctggctagacccggtgatatcttccagataactggacagaaaatcacccaggcaatttttaagaaaatcaacaaaagaaaaatcctccaggtgcacgtttagagcctcgggaacaacgatgaagtaaatgcaagcggtgcgttccagcatggttagttagctgatctgtaaaaaacaaaaaataaaacattaaaccatgctagcctggcgaacaggtgggtaaatcgttctctccagcaccaggcaggccacggggtctccggcgcgaccctcgtaaaaattgtcgctatgattgaaaaccatcacagagagacgttcccggtggccggcgtgaatgattcgacaagatgaatacacccccggaacattggcgtccgcgagtgaaaaaaagcgcccgaggaagcaataaggcactacaatgctcagtctcaagtccagcaaagcgatgccatgcggatgaagcacaaaatcctcaggtgcgtacaaaatgtaattactcccctcctgcacaggcagcgaagcccccgatccctccagatacacatacaaagcctcagcgtccatagcttaccgagcagcagcacacaacaggcgcaagagtcagagaaaggctgagctctaacctgtccacccgctctctgctcaatatatagcccagatctacactgacgtaaaggccaaagtctaaaaatacccgccaaataatcacacacgcccagcacacgcccagaaaccggtgacacactcaaaaaaatacgcgcacttcctcaaacgcccaaactgccgtcatttccgggttcccacgctacgtcatcggaattcgactttcaaattccgtcgaccgttaaaaacgtcacccgccccgcccctaacggtcgcccgtctctcggccaatcaccttcctccctccccaaattcaaacagctcatttgcatattaacgcgcaccaaaagtttgaggtatattattgatgatg